SEQUENCE LISTING (80 sequences)

SEQ ID NO: 1 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - protein, 154 aa, Rattus norvegicus
MFHVSFRYIFGIPPLILVLLPVTSSDCHIKDKDGKAFGSVLMISINQLDKMTGTDSDCPNNEPNFFKKHLCDDTKEAAFLNRAARKLRQFLKMNISEEFNDHLLRVSDGTQTLVNCTSKEEKTIKEQKKNDPCFLKRLLREIKTCWNKILKGSI

SEQ ID NO: 3 - protein, 154 aa, Mus Musculus
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 4 - protein, 177 aa, Cercopithecus aethiops
MFHVSFRYIFGLPPLILVLLPVASSDCDIEGKDGKQYESVLMVSIDQLLDSMKEIGSNCLNNEFNFFKRHLCDDNKEGMFLFRAARKLKQFLKMNSTGDFDLHLLKVSEGTTILLNCTGKVKGRKPAALGEPQPTKSLEENKSLKEQKKLNDSCFLKRLLQKIKTCWNKILMGTKEH

SEQ ID NO: 5 - protein, 176 aa, Bos taurus
MFHVSFRYIFGIPPLILVLLPVASSDCDISGKDGGAYQNVLMVNIDDLDNMINFDSNCLNNEPNFFKKHSCDDNKEASFLNRASRKLRQFLKMNISDDFKLHLSTVSQGTLTLLNCTSKGKGRKPPSLSEAQPTKNLEENKSSKEQKKQNDLCFLKILLQKIKTCWNKILRGIKEH

SEQ ID NO: 6 - protein, 176 aa, Ovis aries
MFHVSFRYIFGIPPLILVLLPVASSDCDFSGKDGGAYQNVLMVSIDDLDNMINFDSNCLNNEPNFFKKHSCDDNKEASFLNRAARKLKQFLKMNISDDFKLHLSTVSQGTLTLLNCTSKGKGRKPPSLGEAQPTKNLEENKSLKEQRKQNDLCFLKILLQKIKTCWNKILRGITEH

SEQ ID NO: 7 - protein, 384 aa, Artificial Sequence
  amino acid sequence of human IgD constant region (Genbank accession No. P01880)
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 8 - protein, 327 aa, Artificial Sequence
  amino acid sequence of Partial human IgG4 constant region (Genbank accession No. AAH25985)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 245 aa, Artificial Sequence
  amino acid sequence of hyFc
RNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 245 aa, Artificial Sequence
  amino acid sequence of hyFcM1
RNTGRGGEEKKGGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 245 aa, Artificial Sequence
  amino acid sequence of hyFcM2
RNTGRGGEEKKGSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 245 aa, Artificial Sequence
  amino acid sequence of hyFcM3
RNTGRGGEEKKSGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 245 aa, Artificial Sequence
  amino acid sequence of hyFcM4
RNTGRGGEEKKSSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 243 aa, Artificial Sequence
  amino acid sequence of mouse IgG Fc variant
ASAEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGNSGS

SEQ ID NO: 15 - protein, 153 aa, Artificial Sequence
  amino acid sequence of modified IL-7(M)
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 16 - protein, 154 aa, Artificial Sequence
  amino acid sequence of modified IL-7(MM)
MMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 17 - protein, 155 aa, Artificial Sequence
  amino acid sequence of modified IL-7(MMM)
MMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 18 - protein, 155 aa, Artificial Sequence
  amino acid sequence of modified IL-7(MGM)
MGMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 19 - protein, 155 aa, Artificial Sequence
  amino acid sequence of modified IL-7(DDD)
DDDDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 20 - protein, 156 aa, Artificial Sequence
  amino acid sequence of modified IL-7(MMMM)
MMMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 21 - protein, 398 aa, Artificial Sequence
  amino acid sequence of modified IL-7(M) fused hyFc
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 399 aa, Artificial Sequence
  amino acid sequence of modified IL-7(MM) fused hyFc
MMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 400 aa, Artificial Sequence
  amino acid sequence of modified IL-7(MMM) fused hyFc
MMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 400 aa, Artificial Sequence
  amino acid sequence of modified IL-7(MGM) fused hyFc
MGMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 401 aa, Artificial Sequence
  amino acid sequence of modified IL-7(MMMM) fused hyFc
MMMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 397 aa, Artificial Sequence
  amino acid sequence of human IL-7 fused hyFc
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 395 aa, Artificial Sequence
  amino acid sequence of human IL-7 fused nonlytic mouse Fc
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHASAEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGNSGS

SEQ ID NO: 28 - DNA, 531 bp, Artificial Sequence
  nucleotide sequence of human IL-7
atgttccacgtgagcttcaggtacatcttcggcctgccacccctgatcctggtgctgctgcctgtggccagctccgactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacac

SEQ ID NO: 29 - DNA, 534 bp, Artificial Sequence
  nucleotide sequence of modified IL-7(M)
atgttccacgtgagcttcagatacatcttcggcctgccccccctgatcctggtgctgctgcccgtggccagcagcatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 30 - DNA, 537 bp, Artificial Sequence
  nucleotide sequence of modified IL-7(MM)
atgttccacgtgagcttcagatacatcttcggcctgccccccctgatcctggtgctgctgcccgtggccagcagcatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 31 - DNA, 540 bp, Artificial Sequence
  nucleotide sequence of modified IL-7(MMM)
atgttccacgtgagcttcagatacatcttcggcctgccccccctgatcctggtgctgctgcccgtggccagcagcatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 32 - DNA, 540 bp, Artificial Sequence
  nucleotide sequence of modified IL-7(MGM)
atgttccacgtgagcttcaggtacatcttcggcctgccacccctgatcctggtgctgctgcctgtggccagctccatggggatggactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcaccaaggagcac

SEQ ID NO: 33 - DNA, 540 bp, Artificial Sequence
  nucleotide sequence of modified IL-7(DDD)
atgttccacgtgagcttcagatacatcttcggcctgccccccctgatcctggtgctgctgcccgtggccagcagcgacgatgacgactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 34 - DNA, 543 bp, Artificial Sequence
  nucleotide sequence of modified IL-7(MMMM)
atgttccacgtgagcttcagatacatcttcggcctgccccccctgatcctggtgctgctgcccgtggccagcagcatgatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 35 - DNA, 1284 bp, Artificial Sequence
  nucleotide sequence of modified IL-7(M) fused hyFc
atgttccacgtgagcttcagatacatcttcggcctgccccccctgatcctggtgctgctgcccgtggccagcagcatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtgactcgagtctaga

SEQ ID NO: 36 - DNA, 1272 bp, Artificial Sequence
  nucleotide sequence of modified IL-7(MM) fused hyFc
atgttccacgtgagcttcagatacatcttcggcctgccccccctgatcctggtgctgctgcccgtggccagcagcatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 37 - DNA, 1275 bp, Artificial Sequence
  nucleotide sequence of modified IL-7(MMM) fused hyFc
atgttccacgtgagcttcagatacatcttcggcctgccccccctgatcctggtgctgctgcccgtggccagcagcatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 38 - DNA, 1275 bp, Artificial Sequence
  nucleotide sequence of modified IL-7(MGM) fused hyFc
atgttccacgtgagcttcaggtacatcttcggcctgccacccctgatcctggtgctgctgcctgtggccagctccatggggatggactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacaccgcaatactgggcggggcggggaggaaaagaaaaaggagaaggaaaaggaggaacaggaggaaagagagactaagaccccagaatgtcccagccatactcagcccctgggggtgttcctgtttccccctaaacctaaggataccctgatgatcagcaggacacccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 39 - DNA, 1278 bp, Artificial Sequence
  nucleotide sequence of modified IL-7(MMMM) fused hyFc
atgttccacgtgagcttcagatacatcttcggcctgccccccctgatcctggtgctgctgcccgtggccagcagcatgatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 40 - protein, 4 aa, Artificial Sequence
  synthetic oligopeptide fragment
MMMM

SEQ ID NO: 41 - protein, 4 aa, Artificial Sequence
  synthetic oligopeptide fragment
MGGM

SEQ ID NO: 42 - protein, 4 aa, Artificial Sequence
  synthetic oligopeptide fragment
MMGG

SEQ ID NO: 43 - protein, 4 aa, Artificial Sequence
  synthetic oligopeptide fragment
GGMM

SEQ ID NO: 44 - protein, 4 aa, Artificial Sequence
  synthetic oligopeptide fragment
MGMG

SEQ ID NO: 45 - protein, 4 aa, Artificial Sequence
  synthetic oligopeptide fragment
GMMG

SEQ ID NO: 46 - protein, 4 aa, Artificial Sequence
  synthetic oligopeptide fragment
GGGM

SEQ ID NO: 47 - protein, 4 aa, Artificial Sequence
  synthetic oligopeptide fragment
MGGG

SEQ ID NO: 48 - protein, 4 aa, Artificial Sequence
  synthetic oligopeptide fragment
GMGG

SEQ ID NO: 49 - protein, 4 aa, Artificial Sequence
  synthetic oligopeptide fragment
GGMG

SEQ ID NO: 50 - protein, 5 aa, Artificial Sequence
  synthetic oligopeptide fragment
GGMMM

SEQ ID NO: 51 - protein, 5 aa, Artificial Sequence
  synthetic oligopeptide fragment
GGGMM

SEQ ID NO: 52 - protein, 5 aa, Artificial Sequence
  synthetic oligopeptide fragment
GGGGM

SEQ ID NO: 53 - protein, 5 aa, Artificial Sequence
  synthetic oligopeptide fragment
MGMMM

SEQ ID NO: 54 - protein, 5 aa, Artificial Sequence
  synthetic oligopeptide fragment
MGGMM

SEQ ID NO: 55 - protein, 5 aa, Artificial Sequence
  synthetic oligopeptide fragment
MGGGM

SEQ ID NO: 56 - protein, 5 aa, Artificial Sequence
  synthetic oligopeptide fragment
MMGMM

SEQ ID NO: 57 - protein, 5 aa, Artificial Sequence
  synthetic oligopeptide fragment
MMGGM

SEQ ID NO: 58 - protein, 5 aa, Artificial Sequence
  synthetic oligopeptide fragment
MMGGG

SEQ ID NO: 59 - protein, 5 aa, Artificial Sequence
  synthetic oligopeptide fragment
MMMGM

SEQ ID NO: 60 - protein, 5 aa, Artificial Sequence
  synthetic oligopeptide fragment
MGMGM

SEQ ID NO: 61 - protein, 5 aa, Artificial Sequence
  synthetic oligopeptide fragment
GMGMG

SEQ ID NO: 62 - protein, 5 aa, Artificial Sequence
  synthetic oligopeptide fragment
GMMMG

SEQ ID NO: 63 - protein, 5 aa, Artificial Sequence
  synthetic oligopeptide fragment
GGMGM

SEQ ID NO: 64 - protein, 5 aa, Artificial Sequence
  synthetic oligopeptide fragment
GGMMG

SEQ ID NO: 65 - protein, 5 aa, Artificial Sequence
  synthetic oligopeptide fragment
GMMGM

SEQ ID NO: 66 - protein, 5 aa, Artificial Sequence
  synthetic oligopeptide fragment
MGMMG

SEQ ID NO: 67 - protein, 5 aa, Artificial Sequence
  synthetic oligopeptide fragment
GMGGM

SEQ ID NO: 68 - protein, 5 aa, Artificial Sequence
  synthetic oligopeptide fragment
MMGMG

SEQ ID NO: 69 - protein, 5 aa, Artificial Sequence
  synthetic oligopeptide fragment
GMMGG

SEQ ID NO: 70 - protein, 5 aa, Artificial Sequence
  synthetic oligopeptide fragment
GMGGG

SEQ ID NO: 71 - protein, 5 aa, Artificial Sequence
  synthetic oligopeptide fragment
GGMGG

SEQ ID NO: 72 - protein, 5 aa, Artificial Sequence
  synthetic oligopeptide fragment
GGGGG

SEQ ID NO: 73 - DNA, 18 bp, Artificial Sequence
  Rpl32
gaaactggcggaaaccca

SEQ ID NO: 74 - DNA, 17 bp, Artificial Sequence
  Rpl32
tctggcccttgaacctt

SEQ ID NO: 75 - DNA, 20 bp, Artificial Sequence
  Cxcl12
tttcagatgcttgacgttgg

SEQ ID NO: 76 - DNA, 18 bp, Artificial Sequence
  Cxcl12
gcgctctgcatcagtgac

SEQ ID NO: 77 - DNA, 32 bp, Artificial Sequence
  Kitl
ctcttcaacattaggtcccgagaaagggaaag

SEQ ID NO: 78 - DNA, 31 bp, Artificial Sequence
  Kitl
cttccagtataaggctccaaaagcaaagcca

SEQ ID NO: 79 - DNA, 20 bp, Artificial Sequence
  Vcam1
tcgggcgaaaaatagtcctt

SEQ ID NO: 80 - DNA, 20 bp, Artificial Sequence
  Vcam1
ccggcatatacgagtgtgaa

(19) United States
(12) Patent Application Publication
(10) Pub. No.: US 2023/0398184 A1
(43) Pub. Date: Dec. 14, 2023

(54) METHODS OF INDUCING STEM CELL MOBILIZATION

(71) Applicants: NeoImmuneTech, Inc., Rockville, MD (US); GENEXINE, INC., Seongnam-si, Gyeonggi-do (KR); POSTECH Academy-Industry Foundation, Pohang-si, Gyeongsangbuk-do (KR)

(72) Inventors: Donghoon CHOI, Seongnam-si, (KR); Young Chul SUNG, Seoul (KR); Seung-Woo LEE, Pohang-si (KR); Sora KIM, Pohang-si (KR); Young-Min KIM, Pohang-si (KR)

(73) Assignees: NeoImmuneTech, Inc., Rockville, MD (US); GENEXINE, INC., Seongnam-si, Gyeonggi-do (KR); POSTECH Academy-Industry Foundation, Pohang-si, Gyeongsangbuk-do (KR)

(21) Appl. No.: 18/250,660

(22) PCT Filed: Oct. 25, 2021
(86) PCT No.: PCT/US2021/056506

§ 371 Date: Apr. 26, 2023

Related U.S. Application Data
(60) Provisional application No. 63/105,828, filed on Oct. 26, 2020.

(51) Int. Cl. A61K 38/20; A61K 35/28; A61K 38/19; A61K 31/395

(52) U.S. Cl. CPC A61K 38/2046; A61K 35/28; A61K 38/193; A61K 31/395

(57) ABSTRACT

The present disclosure relates to methods of promoting the mobilization of HSCs to the peripheral blood with an IL-7 protein. In some aspects, the method further comprises administering an additional agent, such as G-CSF and/or AMD3100. In some aspects, the methods disclosed herein are useful in treating a disease or disorder associated with an abnormality of a hematopoietic process.

### CROSS-REFERENCE TO RELATED APPLICATIONS

This PCT application claims the priority benefit of U.S. Provisional Application No. 63/105,828, filed Oct. 26, 2020, which is incorporated herein by reference in its entirety.

### REFERENCE TO SEQUENCE LISTING SUBMITTED ELECTRONICALLY VIA EFS-WEB

The content of the electronically submitted sequence listing in ASCII text file (Name: 4241_015PC01_SeqListing_ST25.txt; Size: 84,877 bytes; and Date of Creation: Oct. 25, 2021) filed with the application is herein incorporated by reference in its entirety.

### BACKGROUND OF THE DISCLOSURE

Hematopoietic stem cells (HSCs) are largely undifferentiated cells with that are capable of self-replicating and differentiating into more specialized blood cells, such as erythrocytes and immune cells (e.g., lymphocytes). HSCs are primarily found in the bone marrow (e.g., of the pelvis, femur, and sternum), the umbilical cord, and, in small amounts, the peripheral blood. Through hematopoiesis, HSCs play an essential role in the continuous lifelong replenishment of blood cells and the regenerative process of various tissues and organs.

HSC transplantation is a commonly used medical procedure to treat various ailments associated with impaired HSC function, such as that observed in nearly all cancer patients treated with chemotherapy or radiation therapy. In principle, there are two primary methods for collecting the HSCs for transplantation: (i) by repeated aspiration of bone marrow from the pelvic crest or (ii) by leukapheresis after mobilization of the HSCs into the peripheral blood. The latter method is generally favored and considered as the standard, because it is less stressful for the patient and leads to faster engraftment and hematologic reconstitution which can improve patient outcomes. Bazinet et al., Curr Oncol 26(3): 187-191 (June 2019).

Because there are generally very few HSCs circulating in the peripheral blood, the mobilization of HSCs from the bone marrow to the peripheral blood is an essential aspect of HSC transplantation. The most common mobilizing agent for clinical uses is granulocyte colony stimulating factor (G-CSF), alone or in combination with other agents such as AMD3100. Other molecules have mobilizing effects on bone marrow cells (e.g., IL-8 and GM-CSF), but their effects are indirect and not HSC specific. Moreover, even with such mobilizing agents, successful mobilization of the HSCs does not occur and inadequate number of HSCs are harvested from many patients. In such cases, multiple rounds of leukapheresis can be required, which can be extremely stressful for the patients and still not result in the recovery of sufficient number of HSCs. Accordingly, there remains a need for a more effective method of mobilizing HSCs from the bone marrow into the peripheral blood.

### SUMMARY OF THE DISCLOSURE

Provided herein is a method for mobilizing a population of hematopoietic stem and progenitor (LSK) cells from a bone marrow into a peripheral blood of a subject in need thereof, comprising administering to the subject an effective amount of an IL-7 protein.

In some aspects, the administering of the IL-7 protein increases the mobilization of the population of LSK cells from the bone marrow into the peripheral blood. In certain aspects, the increase in the mobilization of the population of LSK cells results in an increase in the number of LSK cells in the peripheral blood of the subject. In some aspects, the number of LSK cells in the peripheral blood of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, or at least about 100-fold or more, compared to a reference (e.g., number of LSK cells in the peripheral blood of a corresponding subject that did not receive an administration of the IL-7 protein).

Also disclosed herein is a method for increasing a mobilization of a population of hematopoietic stem and progenitor (LSK) cells from a bone marrow into a peripheral blood of a subject in need thereof, comprising administering to the subject an effective amount of an IL-7 protein in combination with an additional agent.

In some aspects, the additional agent comprises a G-CSF, CXCR4 antagonist (e.g., AMD3100, POL6326, TG-0054, LY2510924, ALX-0651), CXCR2 antagonist (e.g., bortezomib, Groβ), anti-SDF-1 (e.g., BKT140), GM-CSF, IL-3, GM-CSF/IL-3 fusion proteins, FLK-2/FLT-3 ligand (e.g., CDX-301), stem cell factor, IL-6, IL-11, TPO, VEGF, VLA-4 antagonist (e.g., natalizumab), non-steroidal anti-inflammatory drug (e.g., meloxicam), PTH receptor agonist, TPO receptor agonist (e.g., eltrombopag) plerixafor, chemotherapy, or combinations thereof. In certain aspects, the additional agent comprises G-CSF, AMD3100, or both.

In some aspects, the IL-7 protein and the additional agent are administered concurrently. In some aspects, wherein the IL-7 protein and the additional agent are administered sequentially. In certain aspects, the IL-7 protein is administered prior to the administration of the additional agent. In some aspects, the IL-7 protein is administered after the administration of the additional agent.

In some aspects, in a method for increasing mobilization of LSK cells disclosed herein, the increase in the mobilization of the population of LSK cells results in an increase in the number of LSK cells in the peripheral blood of the subject. In certain aspects, the number of LSK cells in the peripheral blood of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, or at least about 100-fold or more, compared to a reference (e.g., a corresponding subject that received the IL-7 protein alone or the additional agent alone).

In some aspects, LSK cells comprise hematopoietic stem cells (HSCs), short-term HSCs (ST-HSCs), hematopoietic progenitor cell-2 (HPC-2), multipotent progenitors (MPPs), lymphoid-primed progenitor cells (LMPPs), common lymphoid progenitor cells (CLPs), common myeloid progenitor cells (CMPs), granulocyte-monocyte progenitor cells (GMPs), megakaryocyte-erythrocyte progenitor cells (MEPs), or combinations thereof.

In some aspects, the subject of the methods disclosed herein suffers from a tumor. In some aspects, the subject is in need of a HSC transplantation. In some aspects, the subject is a healthy individual.

Present disclosure also provides a method for reconstituting a hematopoietic compartment of a subject having been treated with a therapy that is capable of depleting the hematopoietic compartment of the subject, comprising administering to the subject an effective amount of an IL-7 protein prior to treatment with the therapy, wherein the IL-7 protein is capable of inducing a mobilization of a population of hematopoietic stem and progenitor (LSK) cells from a bone marrow to a peripheral blood of the subject.

In some aspects, the therapy comprises a chemotherapy, radiation therapy, or both.

In some aspects, the method for reconstituting a hematopoietic compartment of a subject provided herein further comprises administering an additional agent to the subject prior to treatment with the therapy. In certain aspects, the additional agent comprises a G-CSF, CXCR4 antagonist (e.g., AMD3100, POL6326, TG-0054, LY2510924, ALX-0651), CXCR2 antagonist (e.g., bortezomib, Groβ), anti-SDF-1 (e.g., BKT140), GM-CSF, IL-3, GM-CSF/IL-3 fusion proteins, FLK-2/FLT-3 ligand (e.g., CDX-301), stem cell factor, IL-6, IL-11, TPO, VEGF, VLA-4 antagonist (e.g., natalizumab), non-steroidal anti-inflammatory drug (e.g., meloxicam), PTH receptor agonist, TPO receptor agonist (e.g., eltrombopag) plerixafor, chemotherapy, or combinations thereof. In some aspects, the additional agent comprises G-CSF, AMD3100, or both.

In some aspects, the IL-7 protein and the additional agent are administered concurrently. In some aspects, the IL-7 protein and the additional agent are administered sequentially. In some aspects, the IL-7 protein is administered prior to the administration of the additional agent. In some aspects, the IL-7 protein is administered after the administration of the additional agent.

In some aspects, the method for reconstituting a hematopoietic compartment of a subject provided herein further comprises isolating the population of LSK cells that have mobilized into the peripheral blood prior to treatment with the therapy. In certain aspects, the isolated population of LSK cells are further expanded ex vivo.

In some aspects, the method for reconstituting a hematopoietic compartment of a subject provided herein additionally comprises infusing the isolated population of LSK cells to the subject after treatment with the therapy, wherein the infusion of the isolated population of LSK cells is capable of reconstituting the hematopoietic compartment of the subject.

In some aspects, the LSK cells comprise hematopoietic stem cells (HSCs), short-term HSCs (ST-HSCs), hematopoietic progenitor cell-2 (HPC-2), multipotent progenitors (MPPs), lymphoid-primed progenitor cells (LMPPs), common lymphoid progenitor cells (CLPs), common myeloid progenitor cells (CMPs), granulocyte-monocyte progenitor cells (GMPs), megakaryocyte-erythrocyte progenitor cells (MEPs), or combinations thereof.

In any of the methods disclosed herein, in some aspects, the population of LSK cells that have mobilized into the peripheral blood are capable of long-term self-renewal. In some aspects, the population of LSK cells that have mobilized into the peripheral blood maintain the ability to self-renew for at least about one week, at least about two weeks, at least about three weeks, at least about one month, at least about two months, at least about three months, at least about four months, at least about five months, at least about six months, at least about seven months, at least about eight months, at least about nine months, at least about 10 months, at least about 11 months, or at least about one year or more.

In any of the methods disclosed herein, in some aspects, the population of LSK cells that have mobilized into the peripheral blood are capable of differentiating into myeloid cells, lymphoid cells (e.g., T cells and/or B cells), or both.

Present disclosure further provides a method for increasing an amount of hematopoietic stem and progenitor (LSK) cells in a peripheral blood of a subject in need thereof, comprising administering to the subject an effective amount of an IL-7 protein.

In some aspects, the amount of LSK cells in the peripheral blood of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about at least about 50-fold, at least about 75-fold, or at least about 100-fold or more, compared to a reference (e.g., number of LSK cells in the peripheral blood of a corresponding subject that did not receive an administration of the IL-7 protein).

Also disclosed herein is a method for increasing an amount of hematopoietic stem and progenitor (LSK) cells in a peripheral blood of a subject in need thereof, comprising administering to the subject an effective amount of an IL-7 protein in combination with an additional agent.

In some aspects, the additional agent comprises a G-CSF, CXCR4 antagonist (e.g., AMD3100, POL6326, TG-0054, LY2510924, ALX-0651), CXCR2 antagonist (e.g., bortezomib, Groβ), anti-SDF-1 (e.g., BKT140), GM-CSF, IL-3, GM-CSF/IL-3 fusion proteins, FLK-2/FLT-3 ligand (e.g., CDX-301), stem cell factor, IL-6, IL-11, TPO, VEGF, VLA-4 antagonist (e.g., natalizumab), non-steroidal anti-inflammatory drug (e.g., meloxicam), PTH receptor agonist, TPO receptor agonist (e.g., eltrombopag) plerixafor, chemotherapy, or combinations thereof. In certain aspects, the additional agent comprises G-CSF, AMD3100, or both.

In some aspects, the IL-7 protein and the additional agent are administered concurrently. In some aspects, the IL-7 protein and the additional agent are administered sequentially. In some aspects, the IL-7 protein is administered prior to the administration of the additional agent. In some aspects, the IL-7 protein is administered after the administration of the additional agent.

In some aspects, the amount of LSK cells in the peripheral blood of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, or at least about 100-fold or more, compared to a reference (e.g., a corresponding subject that received the IL-7 protein alone or the additional agent alone).

In some aspects, the LSK cells comprise hematopoietic stem cells (HSCs), short-term HSCs (ST-HSCs), hematopoietic progenitor cell-2 (HPC-2), multipotent progenitors (MPPs), lymphoid-primed progenitor cells (LMPPs), common lymphoid progenitor cells (CLPs), common myeloid progenitor cells (CMPs), granulocyte-monocyte progenitor cells (GMPs), megakaryocyte-erythrocyte progenitor cells (MEPs), or combinations thereof.

In some aspects, the subject suffers from a tumor. In some aspects, the subject is in need of a HSC transplant. In some aspects, the subject is a healthy individual.

Disclosed herein is a method of treating a disease or disorder in a subject in need thereof, comprising administering to the subject an IL-7 protein and a therapy, wherein the IL-7 protein is capable of inducing a mobilization of a population of hematopoietic stem and progenitor (LSK) cells from a bone marrow to a peripheral blood of the subject. In certain aspects, the IL-7 protein is administered to the subject prior to the therapy.

In some aspects, an amount of the LSK cells in the peripheral blood of the subject is increased compared to a reference (e.g., a corresponding subject that did not receive an administration of the IL-7 protein). In certain aspects, the amount of LSK cells in the peripheral blood of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, or at least about 100-fold or more, compared to the reference.

In some aspects, a method of treating a disease or disorder disclosed herein further comprises isolating the LSK cells from the peripheral blood of the subject prior to the therapy. In certain aspects, the therapy is capable of depleting and/or reducing the number of one or more blood cells in the subject. In some aspects, the one or more blood cells comprise a myeloid cell, lymphoid cell, or both. In some aspects, the myeloid cell comprises a monocyte, macrophage, dendritic cells, mast cells, neutrophil, basophil, eosinophil, erythrocyte, megakaryocyte, or combinations thereof. In some aspects, the lymphoid cell comprises an innate lymphoid cell, natural killer cell, T lymphocyte, B lymphocyte, or combinations thereof.

In some aspects, the therapy of a method of treating a disease or disorder disclosed herein comprises a chemotherapy, radiation therapy, immunotherapy, serotherapy, targeted therapy (e.g., anti-thymocyte immunoglobulin), or combinations thereof.

In some aspects, a method of treating a disease or disorder disclosed herein further comprises infusing the isolated LSK cells to the subject after the administration of the therapy. In certain aspects, the LSK cells comprise hematopoietic stem cells (HSCs), short-term HSCs (ST-HSCs), hematopoietic progenitor cell-2 (HPC-2), multipotent progenitors (MPPs), lymphoid-primed progenitor cells (LMPPs), common lymphoid progenitor cells (CLPs), common myeloid progenitor cells (CMPs), granulocyte-monocyte progenitor cells (GMPs), megakaryocyte-erythrocyte progenitor cells (MEPs), or combinations thereof.

In some aspects, a disease or disorder that can be treated with a method disclosed herein comprises an acute myeloid leukemia, acute lymphoblastic leukemia, chronic myelogenous leukemia, chronic lymphocytic leukemia, myelofibrosis, myelodysplastic syndromes, diffuse large B cell lymphoma, follicular lymphoma, mantle cell lymphoma, Waldenström macroglobulinemia, peripheral T cell lymphoma, primary cutaneous T cell lymphoma, Hodgkin lymphoma, multiple myeloma, amyloidosis, juvenile myelomonocytic leukemia, Non-Hodgkin lymphoma, breast cancer, germ cell tumors, ovarian cancer, medulloblastoma, small cell lung cancer, soft tissue sarcoma, Ewing's sarcoma, renal cell cancer, pancreatic cancer, colorectal cancer, multiple sclerosis, systemic sclerosis, systemic lupus erythematosus, Crohn's disease, rheumatoid arthritis, juvenile idiopathic arthritis, monogenic autoimmune disorders, vasculitis, polymyositis-dermatomyositis, autoimmune cytopenias, neuromyelitis optica, chronic inflammatory demyelinating polyneuropathy, myasthenia gravis and stiff person syndrome, type 1 diabetes, refractory coeliac disease type II, sarcoma, neuroblastoma, brain tumors, Wilms' tumor, or any combination thereof.

In any of the methods disclosed herein, in some aspects, the subject is a human.

In some aspects, the IL-7 protein of any of the methods disclosed herein is not a wild-type IL-7 protein. In certain aspects, the IL-7 protein is a fusion protein.

In some aspects, the IL-7 protein comprises an oligopeptide consisting of 1 to 10 amino acid residues. In some aspects, the oligopeptide comprises methionine (M), glycine (G), methionine-methionine (MM), glycine-glycine (GG), methionine-glycine (MG), glycine-methionine (GM), methionine-methionine-methionine (MMM), methionine-methionine-glycine (MMG), methionine-glycine-methionine (MGM), glycine-methionine-methionine (GMM), methionine-glycine-glycine (MGG), glycine-methionine-glycine (GMG), glycine-glycine-methionine (GGM), glycine-glycine-glycine (GGG), methionine-glycine-glycine-methionine (MGGM) (SEQ ID NO: 41), methionine-methionine-glycine-glycine (MMGG) (SEQ ID NO: 42), glycine-glycine-methionine-methionine (GGMM) (SEQ ID NO: 43), methionine-glycine-methionine-glycine (MGMG) (SEQ ID NO: 44), glycine-methionine-methionine-glycine (GMMG) (SEQ ID NO: 45), glycine-glycine-glycine-methionine (GGGM) (SEQ ID NO: 46), methionine-glycine-glycine-glycine (MGGG) (SEQ ID NO: 47), glycine-methionine-glycine-glycine (GMGG) (SEQ ID NO: 48), glycine-glycine-methionine-glycine (GGMG) (SEQ ID NO: 49), glycine-glycine-methionine-methionine-methionine (GGMMM) (SEQ ID NO: 50), glycine-glycine-glycine-methionine-methionine (GGGMM) (SEQ ID NO: 51), glycine-glycine-glycine-glycine-methionine (GGGGM) (SEQ ID NO: 52), methionine-glycine-methionine-methionine-methionine (MGMMM) (SEQ ID NO: 53), methionine-glycine-glycine-methionine-methionine (MGGMM) (SEQ ID NO: 54), methionine-glycine-glycine-glycine-methionine (MGGGM) (SEQ ID NO: 55), methionine-methionine-glycine-methionine-methionine (MMGMM) (SEQ ID NO: 56), methionine-methionine-glycine-glycine-methionine (MMGGM) (SEQ ID NO: 57), methionine-methionine-glycine-glycine-glycine (MMGGG) (SEQ ID NO: 58), methionine-methionine-methionine-glycine-methionine (MMMGM) (SEQ ID NO: 59), methionine-glycine-methionine-glycine-methionine (MGMGM) (SEQ ID NO: 60), glycine-methionine-glycine-methionine-glycine (GMGMG) (SEQ ID NO: 61), glycine-methionine-methionine-methionine-glycine (GMMMG) (SEQ ID NO: 62), glycine-glycine-methionine-glycine-methionine (GGMGM) (SEQ ID NO: 63), glycine-glycine-methionine-methionine-glycine (GGMMG) (SEQ ID NO: 64), glycine-methionine-methionine-glycine-methionine (GMMGM) (SEQ ID NO: 65), methionine-glycine-methionine-methionine-glycine (MGMMG) (SEQ ID NO: 66), glycine-methionine-glycine-glycine-methionine (GMGGM) (SEQ ID NO: 67), methionine-methionine-glycine-methionine-glycine (MMGMG) (SEQ ID NO: 68), glycine-methionine-methionine-glycine-glycine (GMMGG) (SEQ ID NO: 69), glycine-methionine-glycine-glycine-glycine (GMGGG) (SEQ ID NO: 70), glycine-glycine-methionine-glycine-glycine (GGMGG) (SEQ ID NO: 71), glycine-glycine-glycine-glycine-glycine (GGGGG) (SEQ ID NO: 72), or combinations thereof. In certain aspects, the oligopeptide is methionine-glycine-methionine (MGM).

In some aspects, the IL-7 protein of any of the methods disclosed herein comprises a half-life extending moiety. In certain aspects, the half-life extending moiety comprises an Fc, albumin, an albumin-binding polypeptide, Pro/Ala/Ser (PAS), a C-terminal peptide (CTP) of the β subunit of human chorionic gonadotropin, polyethylene glycol (PEG), long unstructured hydrophilic sequences of amino acids (XTEN), hydroxyethyl starch (HES), an albumin-binding small molecule, or a combination thereof.

In some aspects, the half-life extending moiety is an Fc. In some aspects, the Fc is a hybrid Fc, comprising a hinge region, a CH2 domain, and a CH3 domain, wherein the hinge region comprises a human IgD hinge region, wherein the CH2 domain comprises a part of human IgD CH2 domain and a part of human IgG4 CH2 domain, and wherein the CH3 domain comprises a part of human IgG4 CH3 domain.

In some aspects, the IL-7 protein of any of the methods disclosed herein comprises an amino acid sequence having a sequence identity of at least about 70%, at least about 75%, at least about 80%, at least about 85%, at least about 90%, at least about 95%, at least about 96%, at least about 97%, at least about 98%, at least about 99%, or about 100% to SEQ ID NOs: 1-6 and 15-25.

In some aspects, methods of the present disclosure comprises administering the IL-7 protein at a dose between about 20 μg/kg and about 600 μg/kg. In certain aspects, the IL-7 protein is administered at a dose of about 60 μg/kg. In some aspects, the IL-7 protein is administered at a dosing frequency of about once a week, about once in two weeks, about once in three weeks, about once in four weeks, about once in five weeks, about once in six weeks, about once in seven weeks, about once in eight weeks, about once in nine weeks, about once in 10 weeks, about once in 11 weeks, or about once in 12 weeks. In some aspects, the IL-7 protein is administered to the subject parenthetically, intramuscularly, subcutaneously, ophthalmic, intravenously, intraperitoneally, intradermally, intraorbitally, intracerebrally, intracranially, intraspinally, intraventricular, intrathecally, intracistemally, intracapsularly, intratumorally, or any combination thereof.

In any of the methods disclosed herein, in some aspects, the method further comprises administering at least one additional therapeutic agent to the subject.

### BRIEF DESCRIPTION OF THE FIGURES

FIGS. 1A, 1B, and 1C show the kinetics of HSC and progenitor (LSK) cell mobilization into peripheral blood after a single dose of IL-7 protein (2.5 mg/kg) administration in C57BL/6 mice. FIG. 1A shows a representative dot plot of HSCs (bottom row) and LSK cells (top row) in the peripheral blood of mice treated with either the control buffer (left column) or IL-7 protein (right column) at day 3 post-administration. LSK cells were identified by first gating on the cells that lack the lineage markers (CD3ε, CD11b, B220, CD19, TER-119, NK1.1, WWII, and Gr-1) (i.e., LIN⁻ cells), and then on c-kit⁺ and Sca-1⁺ cells (represented by the boxed region). HSCs were identified by first gating on the LSK cells, and then as CD150⁺ and CD48⁻ (represented by the boxed region). FIG. 1B shows the number of LSK and HSC cells in the peripheral blood of mice treated with a single dose of the IL-7 protein at various time points post-administration. In FIG. 1B, data are shown individually and as mean±S.E.M. FIG. 1C provides the p-values for the results shown in FIG. 1B.

FIGS. 2A, 2B, and 2C show the kinetics of HSC and progenitor (LSK) cell mobilization from the bone marrow after a single dose of IL-7 protein (2.5 mg/kg) administration in C57BL/6 mice. FIG. 2A shows a representative dot plot of HSCs (bottom row) and LSK cells (top row) in the bone marrow of mice treated with either the control buffer (left column) or IL-7 protein (right column) at day 3 post-administration. LSK cells were identified by first gating on the cells that lack the lineage markers (CD3ε, CD11b, B220, CD19, TER-119, NK1.1, MHCII, and Gr-1) (i.e., LIN⁻ cells), and then on c-kit⁺ and Sca-1⁺ (represented by the boxed region). HSCs were identified by first gating on the LSK cells, and then as CD150⁺ and CD48⁻ (represented by the boxed region). FIG. 2B shows the number of LSK and HSC cells in the bone marrow of mice treated with a single dose of the IL-7 protein at various time points post-administration. In FIG. 2B, data are shown individually and as mean±S.E.M. FIG. 2C provides the p-values for the results shown in FIG. 2B.

FIGS. 3A and 3B show the number of progenitor (LSK) and HSC cells in the peripheral blood and bone marrow, respectively, of mice treated with different concentrations of the IL-7 protein at day 3 post administration. A single dose of IL-7 protein was administered to the mice at one of the following concentrations: (i) 0 mg/kg, (ii) 0.1 mg/kg, (iii) 0.5 mg/kg, (iv) 2.5 mg/kg, or (v) 12.5 mg/kg. Data are shown individually and as mean±S.E.M. FIGS. 3C and 3D provide the p-values for the results shown in FIGS. 3A and 3B, respectively.

FIGS. 4A and 4B show the number of different hematopoietic stem and progenitor (LSK) subsets in the peripheral blood (FIG. 4A) and bone marrow (FIG. 4B) of mice treated with either a control buffer (white bars) or the IL-7 protein (black bars). The different hematopoietic stem and progenitor (LSK) cell subsets shown include: (i) (“HSC”), (ii) short-term HSCs (“ST-HSC”), (iii) hematopoietic progenitor cell-2 (“HPC-2”), and (iv) multipotent progenitors (“MPPs”). Total LSK cells are also shown for comparison purposes (see bar graph to the left in both FIGS. 4A and 4B). Data are shown as mean±S.E.M. FIGS. 4C and 4D provide the p-values for the results shown in FIGS. 4A and 4B, respectively.

FIGS. 5A and 5B provides a comparison of the percentage of different types of blood cells present in the peripheral blood of lethally irradiated mice at eight weeks after transplantation with (i) bone marrow (BM) cells from normal (i.e., control) mice (FIG. 5A) or (ii) PBMCs isolated from IL-7 treated mice (FIG. 5B). The different types of blood cells shown include: T cells (“T”), B cells (“B”), and myeloid cells (“M”).

FIG. 6A shows the survival curves for lethally irradiated recipient mice transplanted with PBMCs isolated from donor mice treated with a control buffer (“1”) or from donor mice treated with a single administration of the IL-7 protein (“2”) (i.e. primary transfer). In FIG. 6B, bone marrow cells from the surviving mice in FIG. 6A (i.e., all were transplanted with cells from donor mice treated with IL-7 protein) were transplanted into new lethally irradiated recipient mice (i.e., secondary transfer). “**” indicates a statistically significant difference (p=0.006) compared to control. FIG. 6B provides the survival curve of these mice. In FIG. 6C, bone marrow cells from the surviving mice in FIG. 6B were isolated and transplanted into new lethally irradiated recipient mice (i.e., tertiary transfer). FIG. 6C provides the survival curve of these mice.

FIG. 7A shows the number of CD34+ HSCs in the peripheral blood of healthy human subjects treated (via subcutaneous administration) with either placebo (left graph) or a single dose (60 μg/kg) of the IL-7 protein (right graph). The number of CD34+ HSCs at day 0 (i.e., just before IL-7 protein administration) and at day 10 post IL-7 protein administration are provided. Data are shown as mean±S.E.M. FIG. 7B provides the p-values for the results shown in FIG. 7A.

FIGS. 8A and 8B show the number of LSK cells and HSCs in the peripheral blood of normal (wild-type) and RAG-1 knockout mice, respectively, at day 3 post treatment with either the control buffer (white bar) or IL-7 protein (black bar). Data are shown as mean±S.E.M. FIG. 8C provides the p-values for the results shown in FIGS. 8A and 8B.

FIGS. 9A and 9B show the number of different B cell subsets in the bone marrow of normal (wild-type) and RAG-1 knockout mice, respectively, at day 3 post treatment with either the control buffer (white bar) or IL-7 protein (black bar). Data are shown as mean±S.E.M. FIG. 9C provides the p-values for the results shown in FIGS. 9A and 9B.

FIG. 10 shows the correlation between the number of HSCs present in the peripheral blood and the percentage of proB cells present in the bone marrow of normal mice at day 3 post treatment with a single administration of the IL-7 protein. The IL-7 protein was administered at one of the following concentrations: (i) 0 mg/kg, (ii) 0.1 mg/kg, (iii) 0.5 mg/kg, (iv) 2.5 mg/kg, or (v) 12.5 mg/kg. Each of the circles represent an individual mice.

FIG. 11 shows the number of proB cells in the bone marrow of normal (wild-type) (“circle”) and proB-specific IL-7R deficient mice (“triangle”) at day 3 post treatment with either the control buffer or IL-7 protein. Data are shown individually and as mean±S.E.M. “*” indicates a statistically significant difference (p=0.004) compared to the control animals.

FIGS. 12A and 12B show the mobilization of LSK cells and HSCs into the peripheral blood in normal (wild-type) (“Mb-1^{c/+} IL-7R^{+/+}”) and proB-specific IL-7R deficient mice (“Mb-1^{c/+} IL-7R^{f/f}”) at day 3 post treatment with either the control buffer or IL-7 protein. FIG. 12A shows a representative dot plot of HSCs (bottom row) and LSK cells (top row) observed in the peripheral blood of the different animals. FIG. 12B shows the number of LSK cells and HSCs observed in the peripheral blood of the different animals. In FIG. 12B, data are shown individually and as mean±S.E.M. FIG. 12C provides the p-values for the results shown in FIG. 12B.

FIGS. 13A and 13B show the mobilization of LSK cells and HCS from the bone marrow in normal (wild-type) (“Mb-1^{c/+} IL-7R^{+/+}”) and proB-specific IL-7R deficient mice (“Mb-1^{c/+} IL-7R^{f/f}”) at day 3 post treatment with either the control buffer or IL-7 protein. FIG. 13A shows a representative dot plot of HSCs (bottom row) and LSK cells (top row) observed in the bone marrow of the different animals. FIG. 13B shows the number of LSK cells and HSCs observed in the bone marrow of the different animals. In FIG. 13B, data are shown individually and as mean±S.E.M. FIG. 13C provides the p-values for the results shown in FIG. 13B.

FIGS. 14A, 14B, 14C, 14D, 14E, and 14F show the effect of IL-7 administration on the expression of different niche factors in the bone marrow of mice at day 2 post administration. FIG. 14A shows the relative mRNA expression of three different genes related to stem cell retention on CD45⁻TER119⁻ non-hematopoietic cells (i.e., Cxcl12, Scf, and Vcam1) in normal mice treated with either the control buffer or IL-7 protein. FIG. 14C shows the expression of CXCR4, KIT, and VLA-4 on HSCs from normal mice treated with either the control buffer or IL-7 protein, as measured using flow cytometry (expression is shown as median fluorescence intensity (MFI)). FIG. 14E provides a comparison of VLA-4 expression on HSCs from normal (wild-type) (“Mb-1^{c/+} IL-7R^{+/+}”) and proB-specific IL-7R deficient mice (“Mb-1^{c/+} IL-7R^{f/f}”) treated with either the control buffer or IL-7 protein. VLA-4 expression was measured using flow cytometry (expression is shown as median fluorescence intensity (MFI)). FIGS. 14B, 14D and 14F provide the p-values for the results shown in FIGS. 14A, 14C, and 14E, respectively.

FIGS. 15A, 15B, and 15C provide a comparison of the mobilization of HSCs to the peripheral blood in mice treated with either IL-7 protein or G-CSF. Two different versions of G-CSF were administered to mice: (i) pegylated recombinant human G-CSF (“PEG-rhG-CSF”) or (ii) non-pegylated recombinant human G-CSF (“rhG-CSF”). Mice treated with the control buffer were used as control. FIG. 15A show representative dot plots of HSCs (bottom row) and LSK cells (top row) present in the peripheral blood of the different animals. FIG. 15B shows the number of LSK cells and HSCs, respectively, in the peripheral blood of animals from the different treatment groups. In FIG. 15B, data are shown individually and as mean±S.E.M. FIG. 15C provide the p-values for the results shown in FIG. 15B.

FIGS. 16A, 16B, 16C, 16D, and 16E provide a comparison of the repopulating capacity of PBMCs isolated from mice treated with the IL-7 protein or pegylated recombinant human G-CSF (“G-CSF”) when administered to lethally irradiated recipient mice. FIG. 16A provides a schematic of the experimental design. FIG. 16B shows the percentage of leukocytes in the peripheral blood of recipient mice at weeks 8, 12, and 18 post HSC transplantation. Data are shown as mean±S.E.M. FIG. 16C provides the p-values for the results shown in FIG. 16B. FIG. 16D shows the percentage of different cell populations in the peripheral blood of the recipient mice. The cell populations shown include B cells (B220+), myeloid cells (CD11b+), and T cells (CD3e+). Data are shown individually and as mean±S.E.M. FIG. 16E provides the p-values for the results shown in FIG. 16D.

FIG. 17 provides a comparison of the number of HSCs that mobilized to the peripheral blood in mice at day 3 post treatment with (i) control buffer, (ii) IL-7 protein alone, (iii) pegylated recombinant human G-CSF (“G-CSF”), or (iv) G-CSF in combination with AMD3100. Data are shown individually and as mean±S.E.M.

FIGS. 18A, 18B, 18C, and 18D show the mobilization of HSCs to the peripheral blood after administration of an IL-7 protein in combination with a pegylated recombinant human G-CSF (“Comb.”). For comparison purposes, some of the mice were treated with one of the following: (i) control buffer, (ii) an IL-7 protein alone (“IL-7”), or (iii) the pegylated recombinant human G-CSF alone (“G-CSF”). FIG. 18A provides a schematic of the experimental design. FIGS. 18B and 18C provide a comparison of the number of LSK cells and HSCs, respectively, present in the peripheral blood of animals from the different treatment groups at day 3 post-administration. In FIGS. 18B and 18C, the data are shown individually and as mean±S.E.M. FIG. 18D provides the p-values for the results shown in FIGS. 18B and 18C.

FIGS. 19A, 19B, 19C, and 19D show the mobilization of HSCs to the peripheral blood after administration of an IL-7 protein in combination with AMD3100 (“Comb.”). For comparison purposes, animals were also treated with one of the following: (i) control buffer, (ii) an IL-7 protein alone (“IL-7”), or (iii) AMD3100 alone (“AMD3100”). FIG. 19A provides a schematic of the experimental design. FIGS. 19B and 19C provide a comparison of the number of LSK cells and HSCs, respectively, present in the peripheral blood of animals from the different treatment groups at day 3 post IL-7 protein administration. In FIGS. 19B and 19C, the data are shown individually and as mean±S.E.M. FIG. 19D provides the p-values for the results shown in FIGS. 19B and 19C.

FIGS. 20A, 20B, 20C, and 20D show the mobilization of HSCs to the peripheral blood after administration of an IL-7 protein in combination with both pegylated recombinant human G-CSF and AMD3100 (“G3”). For comparison purposes, some of the mice were treated with one of the following: (i) IL-7 protein in combination with the G-CSF (“G1”) or (ii) IL-7 protein in combination with AMD3100 (“G2”). FIG. 20A provides a schematic of the experimental design. FIGS. 20B and 20C provide a comparison of the number of LSK cells and HSCs, respectively, present in the peripheral blood of animals from the different treatment groups at day 3 post IL-7 protein administration. In FIGS. 20B and 20C, the data are shown individually and as mean±S.E.M. FIG. 20D provides the p-values for the results shown in FIGS. 20B and 20C.

### DETAILED DESCRIPTION OF THE INVENTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

Throughout this disclosure, the term “a” or “an” entity refers to one or more of that entity; for example, “an antibody,” is understood to represent one or more antibodies. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, “administering” refers to the physical introduction of a therapeutic agent or a composition comprising a therapeutic agent to a subject, using any of the various methods and delivery systems known to those skilled in the art. The different routes of administration for a therapeutic agent described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, intratracheal, pulmonary, subcutaneous, subcuticular, intraarticular, subcapsular, subarachnoid, intraventricle, intravitreal, epidural, and intrasternal injection and infusion, as well as in vivo electroporation. Alternatively, a therapeutic agent described herein can be administered via a non-parenteral route, such as a topical, epidermal, or mucosal route of administration, for example, intranasally, orally, vaginally, rectally, sublingually, or topically. Administering can also be performed, for example, once, a plurality of times, and/or over one or more extended periods.

As used herein, the term “antigen” refers to any natural or synthetic immunogenic substance, such as a protein, peptide, or hapten.

The terms “antibody” and “antibodies” are terms of art and can be used interchangeably herein and refer to a molecule with an antigen binding site that specifically binds an antigen. The terms as used to herein include whole antibodies and any antigen binding fragments (i.e., “antigen-binding portions”) or single chains thereof. An “antibody” refers, in some aspects, to a glycoprotein comprising at least two heavy (H) chains and two light (L) chains inter-connected by disulfide bonds, or an antigen-binding portion thereof. In another aspect, an “antibody” refers to a single chain antibody comprising a single variable domain, e.g., VHH domain. Each heavy chain is comprised of a heavy chain variable region (abbreviated herein as VH) and a heavy chain constant region. In certain naturally-occurring antibodies, the heavy chain constant region is comprised of three domains, CH1, CH2 and CH3. In certain naturally-occurring antibodies, each light chain is comprised of a light chain variable region (abbreviated herein as VL) and a light chain constant region. The light chain constant region is comprised of one domain, CL.

The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1 q) of the classical complement system.

Antibodies typically bind specifically to their cognate antigen with high affinity, reflected by a dissociation constant (K_{D}) of 10⁻⁵ to 10⁻¹¹ M or less. Any K_{D} greater than about 10⁻⁴ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, 10⁻⁸ M or less, 5×10⁻⁹ M or less, or between 10⁻⁸ M and 10⁻¹⁰ M or less, but does not bind with high affinity to unrelated antigens. An antigen is “substantially identical” to a given antigen if it exhibits a high degree of sequence identity to the given antigen, for example, if it exhibits at least 80%, at least 90%, at least 95%, at least 97%, or at least 99% sequence identity to the sequence of the given antigen. By way of example, an antibody that binds specifically to PD-1 can, in certain aspects, also have cross-reactivity with PD-1 antigens from certain primate species (e.g., cynomolgus anti-PD-1 antibody), but cannot cross-react with PD-1 molecules from other species or with a molecule other than PD-1.

An immunoglobulin can be derived from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. In certain aspects, one or more amino acids of the isotype can be mutated to alter effector function. The term “antibody” includes, by way of example, both naturally occurring and non-naturally occurring Abs; monoclonal and polyclonal Abs; chimeric and humanized Abs; human or nonhuman Abs; wholly synthetic Abs; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

An “isolated antibody” refers to an antibody that is substantially free of other antibodies having different antigenic specificities (e.g., an isolated antibody that binds specifically to PD-1 is substantially free of antibodies that bind specifically to antigens other than PD-1). An isolated antibody that binds specifically to PD-1 can, however, have cross-reactivity to other antigens, such as PD-1 molecules from different species. Moreover, an isolated antibody can be substantially free of other cellular material and/or chemicals.

The term “monoclonal antibody” (“mAb”) refers to a non-naturally occurring preparation of antibody molecules of single molecular composition, i.e., antibody molecules whose primary sequences are essentially identical, and which exhibits a single binding specificity and affinity for a particular epitope. A mAb is an example of an isolated antibody. MAbs can be produced by hybridoma, recombinant, transgenic or other techniques known to those skilled in the art.

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the invention can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies and are used synonymously.

A “humanized antibody” refers to an antibody in which some, most or all of the amino acids outside the CDR domains of a non-human antibody are replaced with corresponding amino acids derived from human immunoglobulins. In one aspect of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “antigen-binding portion” of an antibody (also called an “antigen-binding fragment”) refers to one or more fragments of an antibody that retain the ability to bind specifically to the antigen bound by the whole antibody.

As used herein, the terms “specific binding,” “selective binding,” “selectively binds,” and “specifically binds,” refer to antibody binding to an epitope on a predetermined antigen. Typically, the antibody (i) binds with an equilibrium dissociation constant (K_{D}) of approximately less than 10⁻⁴ M, such as approximately less than 10⁻M, 10⁻⁹ M or 10⁻¹⁰ M or even lower when determined by, e.g., surface plasmon resonance (SPR) technology in a BIACORE™ 2000 instrument using the predetermined antigen as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” can comprise one or more polypeptides. Unless otherwise specified, the terms “protein” and “polypeptide” can be used interchangeably.

The term “nucleic acid molecule,” as used herein, is intended to include DNA molecules and RNA molecules. A nucleic acid molecule can be single-stranded or double-stranded, and can be cDNA.

“Conservative amino acid substitutions” refer to substitutions of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In certain aspects, a predicted nonessential amino acid residue in an antibody is replaced with another amino acid residue from the same side chain family. Methods of identifying nucleotide and amino acid conservative substitutions which do not eliminate antigen binding are well-known in the art (see, e.g., Brummell et al., Biochem. 32: 1180-1187 (1993); Kobayashi et al. Protein Eng. 12(10):879-884 (1999); and Burks et al. Proc. Natl. Acad. Sci. USA 94:412-417 (1997)).

For nucleic acids, the term “substantial homology” indicates that two nucleic acids, or designated sequences thereof, when optimally aligned and compared, are identical, with appropriate nucleotide insertions or deletions, in at least about 80% of the nucleotides, at least about 90% to 95%, or at least about 98% to 99.5% of the nucleotides. Alternatively, substantial homology exists when the segments will hybridize under selective hybridization conditions, to the complement of the strand.

For polypeptides, the term “substantial homology” indicates that two polypeptides, or designated sequences thereof, when optimally aligned and compared, are identical, with appropriate amino acid insertions or deletions, in at least about 80% of the amino acids, at least about 90% to 95%, or at least about 98% to 99.5% of the amino acids.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, e.g., as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at worldwideweb.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and)(BLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the)(BLAST program, score=50, wordlength=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g.,)(BLAST and NBLAST) can be used. See worldwideweb.ncbi.nlm.nih.gov.

The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., the other parts of the chromosome) or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

Nucleic acids, e.g., cDNA, can be mutated, in accordance with standard techniques to provide gene sequences. For coding sequences, these mutations, can affect amino acid sequence as desired. In particular, DNA sequences substantially homologous to or derived from native V, D, J, constant, switches and other such sequences described herein are contemplated (where “derived” indicates that a sequence is identical or modified from another sequence).

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”) In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny cannot, in fact, be identical to the parent cell, but are still included within the scope of the term “host cell” as used herein.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

As used herein, the term “mobilizing” LSK cells (e.g., hematopoietic stem cells) or LSK cell (e.g., hematopoietic stem cell) mobilization refers to the recruitment of LSK cells (e.g., hematopoietic stem cells (HSCs)) from a first location (e.g., stem cell niche, e.g., bone marrow) to a second location (e.g., tissue (e.g., peripheral blood) or organ). In some aspects, the first location is the bone marrow and the second location is the peripheral blood. The current methods for mobilizing LSK cells (e.g., HSCs) to the peripheral blood involve the use of granulocyte colony stimulating factor (G-CSF), alone or in combination with other agents. Non-limiting examples of other agents that are used in combination with G-CSF to promote LSK cell (e.g., HSC) mobilization include AMD3100 (a CXCR4 antagonist). Other non-limiting examples of agents that can be used alone or in combination with other agents (e.g., G-CSF) to induce LSK cell (e.g., HSC) mobilization include: plerixafor (e.g., MOZOBIL®), chemotherapy (e.g., cyclophosphamide, etoposide (e.g., TOPOSAR® and ETOPOPHOS®), POL6326 (CXCR4 antagonist), TG-0054 (CXCR4 antagonist), BKT140 (anti-SDF-1), bortezomib (proteasome inhibitor, downregulation of VLA4/VCAM-1 axis) (e.g., VELCADE®), Groβ (CXCR2 agonist, induction of MMP-9 secretion), PTH (PTH receptor agonist, expansion of BM HSC), CDX-301 (FLT3 agonist), LY2510924 (CXCR4 antagonist), natalizumab (VLA-4 antagonist) (e.g., TYSABRI®), meloxicam (Non-steroidal anti-inflammatory drug) (e.g., VIVLODEX®, MOBIC®, COMFORT®), eltrombopag (TPO receptor agonist) (e.g., PROMACTA®), ALX-0651 (Anti-CXCR4 Nanobody), and combinations thereof. See, e.g., Domingues et al., Int J Hematol 105:141-152 (2017); Bakanay et al., Bone Marrow Transplantation 47:1154-1163 (2012), both of which is herein incorporated by reference in its entirety. As demonstrated herein (see, e.g., Example 9), the methods disclosed herein can allow for greater mobilization of LSK cells (e.g., HSCs) to the peripheral blood compared to methods currently available in the art (e.g., administration of G-CSF alone or in combination with AMD3100).

As used herein, the term “hematopoietic stem cells” (HSCs) refers to a subset of multipotent stem cells (i.e., LSK cells) that give rise to all the blood or immune cell types, including myeloid (e.g., monocytes and macrophages, neutrophils, basophils, eosinophils, erythrocytes, megakaryocytes/platelets, dendritic cells, mast cells), and lymphoid lineages (e.g., innate lymphoid cells, T-cells, B-cells, NKT-cells, NK-cells), and having multi-lineage hematopoietic differentiation potential and sustained self-renewal activity. The term “stem cells,” as used herein, refers to cells that retain the ability to renew themselves through mitotic cell division and can differentiate into a diverse range of specialized cell types. In some aspects, the therapeutic effects of the present disclosure on HSCs (e.g., promoting HSC mobilization) can also occur with other LSK subsets (see, e.g., Example 4). Accordingly, unless indicated otherwise, the terms LSK cells and HSCs are used interchangeably in the present disclosure.

The term “self-renewal” refers to the ability of a cell to divide and generate at least one daughter cell with the identical (e.g., self-renewing) characteristics of the parent cell. In some aspects, the second daughter cell can commit to a particular differentiation pathway. For example, in certain aspects, a self-renewing hematopoietic stem cell can divide and forms one daughter stem cell and another daughter cell committed to differentiation in the myeloid or lymphoid pathway. In contrast, a committed progenitor cell has typically lost the self-renewal capacity, and upon cell division produces two daughter cells that display a more differentiated (i.e., restricted) phenotype.

As described herein, hematopoietic stem cells (HSCs) can be identified using various phenotypic markers. In certain aspects, the HSCs relevant for the present disclosure are lineage marker negative, Sca-1-positive, cKit-positive (or “LSK cells”). In some aspects, mouse and/or human HSCs can be further identified based on one or more additional phenotypic markers, e.g., as shown in Table 1. Table 1 (below) provides a list of different hematopoietic stem and progenitor (LSK) cell subsets and their phenotypic markers.

TABLE 1
LSK Cell Subsets and Phenotypic Markers

| Organism | LSK Cell Subset | Phenotypic Markers |
|---|---|---|
| Mouse | HSCs | lin⁻SCA-1⁺c-kit⁺CD48⁻CD150⁺ |
| | Short-term HSCs (ST-HSCs) | lin⁻SCA-1⁺c-kit⁺CD48⁻CD150⁻ |
| | hematopoietic progenitor cell-2 (HPC-2) | lin⁻SCA-1⁺c-kit⁺CD48⁺CD150⁺ |
| | multipotent progenitors (MPPs) | lin⁻SCA-1⁺c-kit⁺CD48⁺CD150⁻ |
| | lymphoid-primed progenitor cells (LMPPs) | lin⁻SCA-1⁺c-kit⁺Flt3^{high} |
| | common lymphoid progenitor cells (CLPs) | lin⁻SCA-1^{int}c-kit^{int}CD127⁺ |
| | common myeloid progenitor cells (CMPs) | lin⁻SCA-1⁻c-kit⁺CD34⁺CD16/32^{low} |
| | granulocyte-monocyte progenitor cells (GMPs) | lin⁻SCA-1⁻c-kit⁺CD34⁺CD16/32^{high} |
| | megakaryocyte-erythrocyte progenitor cells | lin⁻SCA-1⁻c-kit⁺CD34⁻CD16/32^{low} |
| | (MEPs) | |
| Human | HSCs | CD34⁺CD38⁻CD90⁺CD45RA⁻ |
| | MPPs | CD34⁺CD38⁻CD90⁻CD45RA⁻ |
| | CLPs | CD34⁺CD10⁺ |
| | CMPs | CD34⁺CD38⁺CD10⁻CD135⁺CD45RA⁻ |
| | GMPs | CD34⁺CD38⁺CD10⁻CD135⁺CD45RA⁺ |
| | MEPs | CD34⁺CD38⁺CD10⁻CD135⁻CD45RA⁻ |

As used herein, the term “differentiation” or “differentiated” refer to cells that are more specialized in their fate or function than at a previous point in their development, and includes both cells that are terminally differentiated and cells that, although not terminally differentiated, are more specialized than at a previous point in their development. The development of a cell from an uncommitted cell (for example, a stem cell), to a cell with an increasing degree of commitment to a particular differentiated cell type, and finally to a terminally differentiated cell is known as “progressive differentiation” or “progressive commitment.” A cell that is “differentiated” relative to a progenitor cell has one or more phenotypic differences relative to that progenitor cell. Phenotypic differences include, but are not limited to, morphologic differences and differences in gene expression and biological activity, including not only the presence or absence of an expressed marker, but also differences in the amount of a marker and differences in the co-expression patterns of a set of markers. The differentiation state of a cell can be determined using various methods known in the art (e.g., flow cytometry).

A “cancer” refers a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” as used herein refers to primary, metastatic and recurrent cancers.

The terms “anticancer agent” and “anticancer drug,” as used herein, refer to any therapeutic agents (e.g., chemotherapeutic compounds and/or molecular therapeutic compounds), radiation therapies, or surgical interventions, used in the treatment of hyperproliferative diseases such as cancer (e.g., in mammals).

The term “hyperproliferative disease” or “hyperproliferative disorder” as used herein, refers to any condition in which a localized population of proliferating cells in an animal is not governed by the usual limitations of normal growth. Non-limiting examples of hyperproliferative disorders include tumors, neoplasms, lymphomas and the like. A neoplasm is said to be benign if it does not undergo invasion or metastasis and malignant if it does either of these. A “metastatic” cell means that the cell can invade and destroy neighboring body structures. Hyperplasia is a form of cell proliferation involving an increase in cell number in a tissue or organ without significant alteration in structure or function. Metaplasia is a form of controlled cell growth in which one type of fully differentiated cell substitutes for another type of differentiated cell.

The term “fusion protein” refers to proteins created through the joining of two or more genes that originally coded for separate proteins. Translation of this fusion gene results in a single polypeptide or multiple polypeptides with functional properties derived from each of the original proteins. In some aspects, the two or more genes can comprise a substitution, a deletion, and/or an addition in its nucleotide sequence.

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIB) receptor. Various properties of human FcγRs are known in the art. The majority of innate effector cell types coexpress one or more activating FcγR and the inhibitory FcγRIIB, whereas natural killer (NK) cells selectively express one activating Fc receptor (FcγRIII in mice and FcγRIIIA in humans) but not the inhibitory FcγRIIB in mice and humans. Human IgG1 binds to most human Fc receptors and is considered equivalent to murine IgG2a with respect to the types of activating Fc receptors that it binds to.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system. Thus, an Fc region comprises the constant region of an antibody excluding the first constant region immunoglobulin domain (e.g., CH1 or CL). In IgG, IgA and IgD antibody isotypes, the Fc region comprises two identical protein fragments, derived from the second (CH2) and third (CH3) constant domains of the antibody's two heavy chains; IgM and IgE Fc regions comprise three heavy chain constant domains (CH domains 2-4) in each polypeptide chain. For IgG, the Fc region comprises immunoglobulin domains CH2 and CH3 and the hinge between CH1 and CH2 domains. Although the definition of the boundaries of the Fc region of an immunoglobulin heavy chain might vary, as defined herein, the human IgG heavy chain Fc region is defined to stretch from an amino acid residue D221 for IgG1, V222 for IgG2, L221 for IgG3 and P224 for IgG4 to the carboxy-terminus of the heavy chain, wherein the numbering is according to the EU index as in Kabat. The CH2 domain of a human IgG Fc region extends from amino acid 237 to amino acid 340, and the CH3 domain is positioned on C-terminal side of a CH2 domain in an Fc region, i.e., it extends from amino acid 341 to amino acid 447 or 446 (if the C-terminal lysine residue is absent) or 445 (if the C-terminal glycine and lysine residues are absent) of an IgG. As used herein, the Fc region can be a native sequence Fc, including any allotypic variant, or a variant Fc (e.g., a non-naturally occurring Fc). Fc can also refer to this region in isolation or in the context of an Fc-comprising protein polypeptide such as a “binding protein comprising an Fc region,” also referred to as an “Fc fusion protein” (e.g., an antibody or immunoadhesion).

A “native sequence Fc region” or “native sequence Fc” comprises an amino acid sequence that is identical to the amino acid sequence of an Fc region found in nature. Native sequence human Fc regions include a native sequence human IgG1 Fc region; native sequence human IgG2 Fc region; native sequence human IgG3 Fc region; and native sequence human IgG4 Fc region as well as naturally occurring variants thereof. Native sequence Fc include the various allotypes of Fcs (see, e.g., Jefferis et al. (2009) mAbs 1: 1).

Additionally, an Fc (native or variant) of the present invention can be in the form of having native sugar chains, increased sugar chains, or decreased sugar chains compared to the native form, or may be in a deglycosylated form. The immunoglobulin Fc sugar chains can be modified by conventional methods such as a chemical method, an enzymatic method, and a genetic engineering method using a microorganism. The removal of sugar chains from an Fc fragment results in a sharp decrease in binding affinity to the C1q part of the first complement component C1, and a decrease or loss of ADCC or CDC, thereby not inducing any unnecessary immune responses in vivo. In this regard, an immunoglobulin Fc region in a deglycosylated or aglycosylated form may be more suitable to the object of the present invention as a drug carrier. As used herein, the term “deglycosylation” refers to an Fc region in which sugars are removed enzymatically from an Fc fragment. Additionally, the term “aglycosylation” means that an Fc fragment is produced in an unglycosylated form by a prokaryote, and preferably in E. coli.

As used herein, the term “immune response” refers to a biological response within a vertebrate against foreign agents, which response protects the organism against these agents and diseases caused by them. An immune response is mediated by the action of a cell of the immune system (e.g., a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4⁺ or CD8⁺ T cell, or the inhibition of a Treg cell.

An “immunomodulator” or “immunoregulator” refers to an agent, e.g., a component of a signaling pathway, that can be involved in modulating, regulating, or modifying an immune response. “Modulating,” “regulating,” or “modifying” an immune response refers to any alteration in a cell of the immune system or in the activity of such cell (e.g., an effector T cell). Such modulation includes stimulation or suppression of the immune system which can be manifested by an increase or decrease in the number of various cell types, an increase or decrease in the activity of these cells, or any other changes which can occur within the immune system. Both inhibitory and stimulatory immunomodulators have been identified, some of which can have enhanced function in a tumor microenvironment. In preferred aspects, the immunomodulator is located on the surface of a T cell. An “immunomodulatory target” or “immunoregulatory target” is an immunomodulator that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

“Immunostimulating therapy” or “immunostimulatory therapy” refers to a therapy that results in increasing (inducing or enhancing) an immune response in a subject for, e.g., treating cancer.

As used herein, the term “interleukin-7” or “IL-7” refers to IL-7 polypeptides and derivatives and analogs thereof having substantial amino acid sequence identity to wild-type mature mammalian IL-7s and substantially equivalent biological activity, e.g., in standard bioassays or assays of IL-7 receptor binding affinity. For example, IL-7 refers to an amino acid sequence of a recombinant or non-recombinant polypeptide having an amino acid sequence of: i) a native or naturally-occurring allelic variant of an IL-7 polypeptide, ii) a biologically active fragment of an IL-7 polypeptide, iii) a biologically active polypeptide analog of an IL-7 polypeptide, or iv) a biologically active variant of an IL-7 polypeptide. IL-7 polypeptides of the invention can be obtained from any species, e.g., human, cow or sheep. IL-7 nucleic acid and amino acid sequences are well known in the art. For example, the human IL-7 amino acid sequence has a Genbank accession number of P13232 (SEQ ID NO: 1); the mouse IL-7 amino acid sequence has a Genbank accession number of P10168 (SEQ ID NO: 3); the rat IL-7 amino acid sequence has a Genbank accession number of P56478 (SEQ ID NO: 2); the monkey IL-7 amino acid sequence has a Genbank accession number of NP_001279008 (SEQ ID NO: 4); the cow IL-7 amino acid sequence has a Genbank accession number of P26895 (SEQ ID NO: 5); and the sheep IL-7 amino acid sequence has a Genbank accession number of Q28540 (SEQ ID NO: 6). In some aspects, an IL-7 polypeptide of the present disclosure is a variant of an IL-7 protein.

A “variant” of an IL-7 protein is defined as an amino acid sequence that is altered by one or more amino acids. The variant can have “conservative” changes, wherein a substituted amino acid has similar structural or chemical properties, e.g., replacement of leucine with isoleucine. More rarely, a variant can have “nonconservative” changes, e.g., replacement of a glycine with a tryptophan. Similar minor variations can also include amino acid deletions or insertions, or both. Guidance in determining which and how many amino acid residues may be substituted, inserted or deleted without abolishing biological activity can be found using computer programs well known in the art, for example software for molecular modeling or for producing alignments. The variant IL-7 proteins included within the invention include IL-7 proteins that retain IL-7 activity. IL-7 polypeptides which also include additions, substitutions or deletions are also included within the invention as long as the proteins retain substantially equivalent biological IL-7 activity. For example, truncations of IL-7 which retain comparable biological activity as the full length form of the IL-7 protein are included within the invention. The activity of the IL-7 protein can be measured using in vitro cellular proliferation assays such as described in Example 6 below. The activity of IL-7 variants of the invention maintain biological activity of at least 10%, 20%, 40%, 60%, but more preferably 80%, 90%, 95% and even more preferably 99% as compared to wild type IL-7.

Variant IL-7 proteins also include polypeptides that have at least about 70%, 75%, 80%, 85%, 90%, 92%, 95%, 96%, 97%, 98%, 99%, or more sequence identity with wild-type IL-7. To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino acid or nucleic acid sequence). The percent identity between the two sequences is a function of the number of identical positions shared by the sequences (i.e., % homology=# of identical positions/total # of positions.times.100). The determination of percent homology between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and)(BLAST programs of Altschul, et al., (1990) J. Mol. Biol. 215:403-10. BLAST nucleotide searches can be performed with the NBLAST program, score=100, wordlength=12. BLAST protein searches can be performed with the)(BLAST program, score=50, wordlength=3. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g.,)(BLAST and NBLAST) can be used.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject” and “patient” are used interchangeably herein.

The term “therapeutically effective amount,” “therapeutically effective dosage,” “effective amount,” or “effective dosage” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. As described herein, in some aspects, the desired result can be an increase in the mobilization of LSK cells (e.g., HSCs) to the peripheral blood. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations. The effective amount of the drug or composition can: (i) increase the mobilization of LSK cells (e.g., HSCs) to the peripheral blood, (ii) increase the number of LSK cells (e.g., HSCs) present in the peripheral blood, (iii) promote the differentiation of LSK cells (e.g., HSCs) into one or more types of blood cells, (iv) reduce the number of cancer cells; (v) reduce tumor size; (vi) inhibit, retard, slow to some extent and can stop cancer cell infiltration into peripheral organs; (vii) inhibit (i.e., slow to some extent and can stop) tumor metastasis; (viii) inhibit tumor growth; (ix) prevent or delay occurrence and/or recurrence of tumor; and/or (x) relieve to some extent one or more of the symptoms associated with the cancer. The ability of a therapeutic agent disclosed herein (e.g., IL-7 protein, alone or in combination with an additional agent, such as G-CSF and/or AMD3100) to achieve a desired result of the present disclosure (e.g., increased mobilization of LSK cells (e.g., HSCs) to the peripheral blood) can be evaluated using a variety of methods known to the skilled practitioner, such as in human subjects during clinical trials, in animal model systems predictive of efficacy in humans, or by assaying the activity of the agent in in vitro assays.

The term “dosing frequency” refers to the number of times a therapeutic agent (e.g., an IL-7 protein alone or in combination with an additional agent, such as G-CSF and/or AMD3100) is administered to a subject within a specific time period. Dosing frequency can be indicated as the number of doses per a given time, for example, once per day, once a week, or once in two weeks. As used herein, “dosing frequency” is applicable where a subject receives multiple (or repeated) administrations of a therapeutic agent.

As used herein, the term “standard of care” refers to a treatment that is accepted by medical experts as a proper treatment for a certain type of disease and that is widely used by healthcare professionals. The term can be used interchangeable with any of the following terms: “best practice,” “standard medical care,” and “standard therapy.”

As used herein, the term “drug” refers to any bioactive agent (e.g., an IL-7 protein or in combination with an additional agent, such as G-CSF and/or AMD3100) intended for administration to a human or non-human mammal to achieve a desired result disclosed herein (e.g., promote the mobilization of LSK cells (e.g., HSCs) to peripheral blood, and/or to prevent or treat a disease or other undesirable condition). Drugs include hormones, growth factors, proteins, peptides and other compounds. In some aspects, a drug disclosed herein is an anti-cancer agent.

By way of example, an “anti-cancer agent” promotes cancer regression in a subject or prevents further tumor growth. In certain aspects, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example, for the treatment of tumors, a therapeutically effective amount of an anti-cancer agent can inhibit cell growth or tumor growth by at least about 10%, at least about 20%, by at least about 40%, by at least about 60%, or by at least about 80% relative to untreated subjects or, in certain aspects, relative to patients treated with a standard-of-care therapy. In other aspects of the invention, tumor regression can be observed and continue for a period of at least about 20 days, at least about 40 days, or at least about 60 days. Notwithstanding these ultimate measurements of therapeutic effectiveness, evaluation of immunotherapeutic drugs must also make allowance for “immune-related” response patterns.

As used herein, the term “immune checkpoint inhibitor” refers to molecules that totally or partially reduce, inhibit, interfere with or modulate one or more immune checkpoint proteins. Immune checkpoint proteins regulate T-cell activation or function. Numerous checkpoint proteins are known, such as CTLA-4 and its ligands CD80 and CD86; and PD-1 with its ligands PD-L1 and PD-L2. Pardoll, D. M., Nat Rev Cancer 12(4):252-64 (2012). These proteins are responsible for co-stimulatory or inhibitory interactions of T-cell responses. Immune checkpoint proteins regulate and maintain self-tolerance and the duration and amplitude of physiological immune responses. Immune checkpoint inhibitors include antibodies or are derived from antibodies.

The term “reference,” as used herein, refers to a corresponding subject (e.g., a cancer subject) who did not receive an administration of an IL-7 protein disclosed herein (alone or in combination with an additional agent, such as G-CSF and/or AMD3100). In some aspects, the reference subject received neither an IL-7 protein nor an additional agent, such as G-CSF and/or AMD3100. The term “reference” can also refer to a same cancer subject but prior to the administration of the IL-7 protein (alone or in combination with an additional agent disclosed herein). In certain aspects, the term “reference” refers to an average of a population of subjects (e.g., cancer subjects).

As used herein, the terms “ug” and “uM” are used interchangeably with “μg” and “μM,” respectively.

Various aspects described herein are described in further detail in the following subsections.

### II. Methods of the Disclosure

Method of Mobilizing LSK Cells (e.g., HSCs)

The present disclosure is directed to a method of mobilizing hematopoietic stem and progenitor (LSK) cells in a subject in need thereof. In some aspects, the method comprises administering to the subject an effective amount of an interleukin-7 (IL-7) protein, wherein the IL-7 protein is capable of promoting the mobilization of LSK cells (e.g., HSCs) to the peripheral blood of the subject. In certain aspects, LSK cells comprise hematopoietic stem cells (HSCs), short-term HSCs (ST-HSCs), hematopoietic progenitor cell-2 (HPC-2), multipotent progenitors (MPPs), lymphoid-primed progenitor cells (LMPPs), common lymphoid progenitor cells (CLPs), common myeloid progenitor cells (CMPs), granulocyte-monocyte progenitor cells (GMPs), megakaryocyte-erythrocyte progenitor cells (MEPs), or combinations thereof.

As demonstrated herein, in some aspects, administering an IL-7 protein can allow for increased mobilization of LSK cells (e.g., HSCs) to the peripheral blood compared to methods currently available in the art (e.g., administration of G-CSF alone or in combination with AMD3100). In some aspects, the increased mobilization of LSK cells (e.g., HSCs) results in a greater number of LSK cells (e.g., HSCs) present in the peripheral blood of the subject. In certain aspects, the number of LSK cells (e.g., HSCs) present in the peripheral blood of a subject treated with an IL-7 protein disclosed herein is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, or at least about 100-fold or more, compared to a reference. In some aspects, the reference is the number of LSK cells (e.g., HSCs) present in the peripheral blood of the subject prior to the administration of the IL-7 protein. In some aspects, the reference is the number of LSK cells (e.g., HSCs) present in the peripheral blood of a corresponding subject that received an alternative regimen for mobilizing LSK cells (e.g., HSCs), wherein the alternative regimen does not include an IL-7 protein (e.g., G-CSF alone or in combination with AMD3100).

As described herein, hallmarks of many LSK cells (e.g., HSCs) include long-term self-renewal capability and the ability to differentiate into more specialized blood cells. In some aspects, LSK cells (e.g., HSCs) mobilized to the peripheral blood using a method disclosed herein (e.g., administration of an IL-7 protein) retain one or more of the functional features of normal LSK cells (e.g., HSCs). For example, in some aspects, the LSK cells (e.g., HSCs) that mobilize to the peripheral blood after an IL-7 protein administration are capable of self-replicating. As used herein, the term “self-replicating” or “self-renewing” refer to the ability to produce replicate daughter LSK cells (e.g., HSCs) having differentiation potential that is identical to those from which they arose. In certain aspects, the LSK cells (e.g., HSCs) are capable of self-renewing for at least about one week, at least about two weeks, at least about three weeks, at least about four weeks, at least about five weeks, at least about six weeks, at least about seven weeks, at least about eight weeks, at least about nine weeks, at least about ten weeks, at least about 11 weeks, at least about 12 weeks, at least about 13 weeks, at least about 14 weeks, at least about 15 weeks, at least about 20 weeks, at least about 25 weeks, at least about 30 weeks, at least about 35 weeks, at least about 40 weeks, at least about 45 weeks, at least about 50 weeks, at least about one year, at least about two years, at least about three years, at least about four years, or at least about five years or more after mobilizing to the peripheral blood.

In some aspects, the LSK cells (e.g., HSCs) that mobilize to the peripheral blood using a method disclosed herein (e.g., administration of an IL-7 protein) are capable of differentiating into different blood cells. In certain aspects, the LSK cells (e.g., HSCs) are capable of differentiating into a myeloid cell. In certain aspects, the LSK cells (e.g., HSCs) are capable of differentiating into a lymphoid cell. In some aspects, the LSK cells (e.g., HSCs) are capable of differentiating into both myeloid and lymphoid cells. In certain aspects, myeloid cells comprise monocytes, macrophages, dendritic cells, mast cells, neutrophils, basophils, eosinophils, erythrocytes, megakaryocytes, or combinations thereof. In some aspects, lymphoid cells comprise innate lymphoid cells, natural killer cells, T lymphocytes, B lymphocytes, or combinations thereof.

As demonstrated herein, an IL-7 protein disclosed herein can improve the LSK cell (e.g., HSC) mobilization effects observed with regimens currently used in the art (e.g., G-CSF, alone or in combination with AMD3100). Accordingly, in some aspects, the present disclosure is related to a method of increasing the mobilization of LSK cells (e.g., HSCs) to the peripheral blood of a subject in need thereof, comprising administering to the subject an effective amount of an IL-7 protein (e.g., disclosed herein) in combination with an additional agent. As used herein, the term “additional agent” refers to a substance with one or more properties that are useful for inducing the mobilization of LSK cells (e.g., HSCs) to the peripheral blood. In some aspects, the additional agent comprises G-CSF and/or AMD3100, which are commonly used in the art to promote LSK cell (e.g., HSC) mobilization. Non-limiting examples of other additional agents that can be used in combination with an IL-7 protein are provided elsewhere in the present disclosure.

In some aspects, administering an IL-7 protein in combination with an additional agent disclosed herein (e.g., those used in the art for mobilizing LSK cells (e.g., HSCs)) results in greater mobilization of LSK cells (e.g., HSCs), compared to administering the IL-7 protein alone or the additional agent alone. In some aspects, the greater mobilization of LSK cells (e.g., HSCs) results in greater number of LSK cells (e.g., HSCs) present in the peripheral blood of a subject. In certain aspects, the number of LSK cells (e.g., HSCs) present in the peripheral blood of a subject treated with a combination of an IL-7 protein and an additional agent is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, or at least about 100-fold or more, compared to the number of LSK cells (e.g., HSCs) present in a corresponding subject that received the IL-7 protein alone or the additional agent alone.

In some aspects, administering an IL-7 protein in combination with an additional agent disclosed herein can reduce the dose of the IL-7 protein required to mobilize LSK cells (e.g., HSCs) to the peripheral blood. In some aspects, administering an IL-7 protein in combination with an additional agent disclosed herein can reduce the dose of the additional agent required to mobilize LSK cells (e.g., HSCs) to the peripheral blood. In some aspects, administering an IL-7 protein in combination with an additional agent disclosed herein can reduce the dose of both the IL-7 protein and the additional agent required to mobilize LSK cells (e.g., HSCs) to the peripheral blood. For example, in some aspects, when administering an IL-7 protein in combination with an additional agent disclosed herein to mobilize LSK cells (e.g., HSC) to the peripheral blood, the dose of the IL-7 protein can be reduced by at least about 5%, at least about 10%, at least about 20%, at least about 30%, at least about 40%, at least about 50%, at least about 60%, at least about 70%, at least about 80%, or at least about 90% or more. In certain aspects, administering an IL-7 protein in combination with an additional agent disclosed herein can reduce the dose of the IL-7 protein by about 80%.

In some aspects, a method of increasing LSK cell (e.g., HSC) mobilization comprises administering the IL-7 protein (e.g., disclosed herein) in combination with G-CSF. In some aspects, a method of increasing LSK cell (e.g., HSC) mobilization comprises administering the IL-7 protein in combination with AMD3100. In some aspects, a method of increasing LSK cell (e.g., HSC) mobilization comprises administering the IL-7 protein in combination with G-CSF and AMD3100. Non-limiting examples of additional agents that can be used in combination with an IL-7 protein disclosed herein to increase LSK cell (e.g., HSC) mobilization include GM-CSF, IL-3, GM-CSF/IL-3 fusion proteins, FLK-2/FLT-3 ligand, stem cell factor, IL-6, IL-11, TPO, VEGF, plerixafor (e.g., MOZOBIL®), chemotherapy (e.g., cyclophosphamide, etoposide (e.g., TOPOSAR® and ETOPOPHOS®), POL6326 (CXCR4 antagonist), TG-0054 (CXCR4 antagonist), BKT140 (anti-SDF-1), bortezomib (proteasome inhibitor, downregulation of VLA4/VCAM-1 axis) (e.g., VELCADE®), Groβ (CXCR2 agonist, induction of MMP-9 secretion), PTH (PTH receptor agonist, expansion of BM HSC), CDX-301 (FLT3 agonist), LY2510924 (CXCR4 antagonist), natalizumab (VLA-4 antagonist) (e.g., TYSABRI®), meloxicam (Non-steroidal anti-inflammatory drug) (e.g., VIVLODEX®, MOBIC®, COMFORT®), eltrombopag (TPO receptor agonist) (e.g., PROMACTA®), ALX-0651 (Anti-CXCR4 Nanobody), or combinations thereof.

In some aspects, the IL-7 protein and the additional agent can be administered concurrently as a single composition. In certain aspects, the IL-7 protein and the additional agent can be administered concurrently as separate compositions. In some aspects, the IL-7 protein and the additional agent can be administered sequentially. In some aspects, the IL-7 protein is administered to the subject prior to the administration of the additional agent. In certain aspects, the IL-7 protein is administered to the subject after the administration of the additional agent.

Method of Increasing the Recovery of LSK Cells (e.g., HSCs) from Peripheral Blood

As described herein, for successful HSC transplantation, it is imperative that a sufficient number of HSCs are recovered from a donor subject. In an “autologous” HSC transplantation, the LSK cells (e.g., HSCs) are isolated from a subject in need of a treatment (e.g., chemotherapy or radiation therapy) and then administered back to the subject after the treatment. Therefore, in the context of autologous HSC transplantation, the terms “donor” and “subject”/“recipient” refer to the same individual. In a “syngenic” HSC transplantation, LSK cells (e.g., HSCs) are isolated from an identical twin of the subject to be treated and then administered to the subject after treatment (e.g., chemotherapy or radiation therapy). In an “allogeneic” HSC transplantation, LSK cells (e.g., HSCs) are isolated from a healthy volunteer (e.g., non-identical twin or an individual not related to the subject to be treated) and then administered to a different recipient subject after treatment (e.g., chemotherapy or radiation therapy). In such transplantations, the terms “donor” and “subject”/“recipient” refer to different individuals.

Not to be bound by any one theory, in some aspects, increased mobilization of LSK cells (e.g., HSCs) can allow for greater recovery of LSK cells (e.g., HSCs) from the peripheral blood. Accordingly, in some aspects, the present disclosure is related to a method of increasing the amount of LSK cells (e.g., HSCs) isolated from the peripheral blood of a donor subject, comprising administering to the subject an effective amount of an IL-7 protein in combination with an additional agent. As described herein, in some aspects, the donor subject is a healthy individual. In some aspects, a donor subject is suffering from a tumor. In some aspects, a donor subject is in need of a HSC transplantation.

In some aspects, the additional agent that can be administered in combination with an IL-7 protein to increase the recovery of HSCs from peripheral blood comprises G-CSF, CXCR4 antagonist (e.g., AMD3100, POL6326, TG-0054, LY2510924, ALX-0651), CXCR2 antagonist (e.g., bortezomib, Groβ), anti-SDF-1 (e.g., BKT140), GM-CSF, IL-3, GM-CSF/IL-3 fusion proteins, FLK-2/FLT-3 ligand (e.g., CDX-301), stem cell factor, IL-6, IL-11, TPO, VEGF, VLA-4 antagonist (e.g., natalizumab), non-steroidal anti-inflammatory drug (e.g., meloxicam), PTH receptor agonist, TPO receptor agonist (e.g., eltrombopag) plerixafor, chemotherapy, or combinations thereof. In certain aspects, the additional agent is G-CSF. In some aspects, the additional agent is AMD3100. In some aspects, the additional agent is a combination of G-CSF and AMD3100.

In some aspects, the number of LSK cells (e.g., HSCs) isolated from the peripheral blood of the donor subject is increased compared to the number of LSK cells (e.g., HSCs) isolated from the peripheral blood of a reference subject. In certain aspects, the reference subject is the donor subject prior to the administration of the IL-7 protein in combination with the additional agent. In some aspects, the reference subject is a corresponding donor subject that did not receive an administration of the IL-7 protein in combination with the additional agent (e.g., received an administration of the IL-7 protein alone or the additional agent alone). In some aspects, the number of LSK cells (e.g., HSCs) isolated from the peripheral blood of a donor subject treated with the IL-7 protein in combination with the additional agent is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, or at least about 100-fold or more, compared to the reference subject.

As described herein, the IL-7 protein and the additional agent can be administered to the subject concurrently (e.g., as a single composition or separate compositions) or sequentially (e.g., the IL-7 protein can be administered prior to or after the administration of the additional agent).

In some aspects, a method of increasing the amount of LSK cells (e.g., HSCs) isolated from the peripheral blood of a donor subject, further comprises isolating the LSK cells (e.g., HSCs) from the peripheral blood after the administration. The LSK cells (e.g., HSCs) can be isolated from the peripheral blood by any methods known in the art.

Method of Reconstituting a Hematopoietic Compartment

In some aspects, the present disclosure is related to a method of reconstituting a hematopoietic compartment of a subject in need thereof. For example, in some aspects, the method disclosed herein can be used to reconstitute the hematopoietic compartment of a subject that has been treated with a therapy, wherein the therapy is capable of depleting the hematopoietic compartment of the subject. As used herein, the term “hematopoietic compartment” refers to the cell compartment in a subject that contains all blood cell lineages, including without limitation, the myeloid lineage, which includes, without limitation, monocytes, macrophages, mast cells, neutrophils, basophils, eosinophils, erythrocytes, megakaryocytes, platelets, and dendritic cells; and the lymphoid lineage, which includes, without limitation, innate lymphoid cells, T cells, B cells, natural killer T (NKT) cells, and NK cells. The “hematopoietic compartment” can contain all immature, mature, undifferentiated, and differentiated white blood cell populations and sub-populations, including tissue-specific and specialized varieties.

In some aspects, the method of reconstituting a hematopoietic compartment comprises administering to a donor subject an effective amount of an IL-7 protein, wherein the IL-7 protein is capable of inducing the mobilization of the LSK cells (e.g., HSCs) to the peripheral blood of the donor subject. In some aspects, the donor subject is a healthy volunteer. In some aspects, the donor subject is suffering from a tumor. In some aspects, the donor subject is in need of a HSC transplantation. In some aspects, the donor subject is a subject in need of a treatment (e.g., chemotherapy and/or radiation therapy).

As described herein, in some aspects, the mobilization of LSK cells (e.g., HSCs) to the peripheral blood can increase the amount of LSK cells (e.g., HSCs) present in the peripheral blood of the donor subject. In certain aspects, the amount of LSK cells (e.g., HSCs) present in the peripheral blood of the donor subject is increased at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, or at least about 100-fold or more, compared to a subject that did not receive the IL-7 protein administration.

In some aspects, a method of reconstituting a hematopoietic compartment of a subject further comprises isolating the LSK cells (e.g., HSCs) from the peripheral blood of the donor subject (e.g., using any method known in the art). In some aspects, a method of reconstituting a hematopoietic compartment of a subject further comprises administering the isolated LSK cells (e.g., HSCs) to a recipient subject. As described herein, in certain aspects, the recipient subject suffers from a tumor. In some aspects, the recipient subject is in need of a LSK cells (e.g., HSC) transplantation. In some aspects, the recipient subject has been treated with a therapy that is capable of depleting the hematopoietic compartment of the subject. Non-limiting examples of such a therapy includes chemotherapy, radiation therapy, or both.

In certain aspects, the method comprises further expanding the isolated LSK cells (e.g., HSCs) ex vivo prior to administering the isolated LSK cells (e.g., HSCs) to the recipient subject. Methods of expanding the isolated LSK cells (e.g., HSCs) ex vivo are known in the art. See, e.g., Tajer et al., Cells 8(2): 169 (2019); and McNiece et al., Exp Hematol 29(1): 3-11 (2001); each of which is herein incorporated by reference in its entirety. In some aspects, the isolated LSK cells (e.g., HSCs) are expanded ex vivo by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, or at least about 100-fold or more, prior to administering the isolated LSK cells (e.g., HSCs) to the recipient subject.

As described herein, in some aspects, the donor subject and the recipient subject are the same individual (i.e., autologous transplantation). In such aspects, the steps of administering the IL-7 protein and isolating the LSK cells (e.g., HSCs) from the peripheral blood are performed prior to the recipient subject being treated with a therapy that is capable of depleting the hematopoietic compartment of the subject. In some aspects, the therapy comprises a chemotherapy, radiation therapy, or both. In certain aspects, the step of further expanding the isolated LSK cells (e.g., HSCs) ex vivo can be performed prior to, concurrently, or after administering the therapy to the recipient subject.

Accordingly, in some aspects, a method of reconstituting a hematopoietic compartment of a subject having been treated with a therapy that is capable of depleting the hematopoietic compartment of the subject comprises (in the following order): (i) administering an effective amount of an IL-7 protein (e.g., disclosed herein) to the subject prior to the therapy, (ii) isolating the LSK cells (e.g., HSCs) from the peripheral blood of the subject prior to the therapy, and (iii) administering the isolated LSK cells (e.g., HSCs) to the subject after the therapy. In some aspects, the method further comprises expanding the isolated LSK cells (e.g., HSCs) ex vivo prior to administering the isolated LSK cells (e.g., HSCs) to the subject.

In some aspects, administering the isolated LSK cells (e.g., HSCs) described above to a recipient subject increases the number of LSK cells (e.g., HSCs) in the recipient subject, wherein the increased number of LSK cells (e.g., HSCs) are capable of reconstituting the hematopoietic compartment of the recipient subject. In certain aspects, administering the isolated LSK cells (e.g., HSCs) to the recipient subject increases the number of LSK cells (e.g., HSCs) in the recipient subject by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, or at least about 100-fold or more, compared to prior to administering the LSK cells (e.g., HSCs).

As described herein, in some aspects, LSK cells (e.g., HSCs) that are mobilized to the peripheral blood using methods disclosed herein and subsequently administered to a subject to reconstitute a hematopoietic compartment are capable of long-term self-renewal. In certain aspects, after being administered to a subject, the LSK cells (e.g., HSCs) described herein are capable of self-renewing for at least about one week, at least about two weeks, at least about three weeks, at least about four weeks, at least about five weeks, at least about six weeks, at least about seven weeks, at least about eight weeks, at least about nine weeks, at least about ten weeks, at least about 11 weeks, at least about 12 weeks, at least about 13 weeks, at least about 14 weeks, at least about 15 weeks, at least about 20 weeks, at least about 25 weeks, at least about 30 weeks, at least about 35 weeks, at least about 40 weeks, at least about 45 weeks, at least about 50 weeks, at least about one year, at least about two years, at least about three years, at least about four years, or at least about five years or more in the recipient subject.

As described herein, a hallmark of LSK cells (e.g., HSCs) is their ability to differentiate into more specialized blood cells. Accordingly, in some aspects, administering the LSK cells (e.g., HSCs) to a recipient subject can increase the number of blood cells in the recipient subject. In certain aspects, the number of blood cells in the recipient subject is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, or at least about 100-fold or more, compared to the number of blood cells present in the recipient subject prior to administering the LSK cells (e.g., HSCs). In certain aspects, after administering the LSK cells (e.g., HSCs) to a recipient subject, the number of blood cells present in the recipient subject is similar to the number of blood cells that are naturally present in a healthy subject. In some aspects, a blood cell comprises a myeloid cell. In some aspects, a blood cell comprises a lymphoid cell. In some aspects, a blood cell comprises both myeloid and lymphoid cells. In certain aspects, myeloid cells comprise monocytes, macrophages, dendritic cells, mast cells neutrophils, basophils, eosinophils, erythrocytes, megakaryocytes, or combinations thereof. In some aspects, lymphoid cells comprise innate lymphoid cells, natural killer cells, T lymphocytes, B lymphocytes, or combinations thereof.

In some aspects, reconstituting the hematopoietic compartment of a recipient subject using the methods disclosed herein increases the survival of the recipient subject. In certain aspects, the survival of the recipient subject is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, or at least about 100-fold or more, compared to the survival of a corresponding subject that did not receive the LSK cells (e.g., HSCs) disclosed herein. In some aspects, after reconstituting the hematopoietic compartment using a method disclosed herein, the survival of the subject is increased by at least about one week, at least about two weeks, at least about three weeks, at least about four weeks, at least about five weeks, at least about six weeks, at least about seven weeks, at least about eight weeks, at least about nine weeks, at least about ten weeks, at least about 11 weeks, at least about 12 weeks, at least about 13 weeks, at least about 14 weeks, at least about 15 weeks, at least about 20 weeks, at least about 25 weeks, at least about 30 weeks, at least about 35 weeks, at least about 40 weeks, at least about 45 weeks, at least about 50 weeks, at least about one year, at least about two years, at least about three years, at least about four years, or at least about five years or more.

In some aspects, a method of reconstituting a hematopoietic compartment disclosed herein comprises administering the IL-7 protein in combination with an additional agent. In certain aspects, the method comprises administering the IL-7 protein in combination with G-CSF. In some aspects, the method comprises administering the IL-7 protein in combination with AMD3100. In some aspects, the method comprises administering the IL-7 protein in combination with G-CSF and AMD3100. Non-limiting examples of other agents that can be used in combination with an IL-7 protein disclosed herein are provided elsewhere in the present disclosure. In some aspects, the IL-7 protein and the additional agent can be administered to the subject concurrently (e.g., as a single composition or separate compositions) or sequentially (e.g., the IL-7 protein can be administered prior to or after the administration of the additional agent).

Method of Treating

As described herein, an impairment in the hematopoietic process can result in reduced number of blood cells, which are essential for maintaining the health of a subject. As used herein, the term “hematopoietic process” is interchangeable with the term “hematopoiesis” and refers to the continuous, regulated process of renewal, proliferation, differentiation, and maturation of all blood cells from LSK cells (e.g., HSCs). For instance, in some aspects, an impaired hematopoietic process results in reduced number of red blood cells, which can lead to disorders such as anemia. In some aspects, an impaired hematopoietic process results in reduced number of immune cells (e.g., T lymphocytes and B lymphocytes), which could lead to increased susceptibility to infections. Not to be bound by any one theory, in some aspects, the reconstitution of a hematopoietic compartment can improve the health of a subject suffering from an impaired hematopoietic process, e.g., by increasing the number of different blood cells that are essential for good health in the subject. Accordingly, in some aspects, the present disclosure provides a method of treating an abnormality of a hematopoietic process in a subject in need thereof, comprising administering to the subject an effective amount of hematopoietic stem and progenitor (LSK) cells (e.g., HSCs).

As described herein, in some aspects, the LSK cells (e.g., HSCs) are derived from a donor subject (e.g., described herein) who has received one or more doses of an IL-7 protein, such as those described herein. In some aspects, the donor subject is the same as the subject to be treated. Accordingly, in such aspects, a method of treating an abnormality of a hematopoietic process in a subject comprises administering a population of LSK cells (e.g., HSCs) in combination with an IL-7 protein. In certain aspects, the IL-7 protein is administered to the subject prior to the population of LSK cells (e.g., HSCs). As described herein, in some aspects, the method further comprises isolating the population of LSK cells (e.g., HSCs) after the IL-7 protein administration, and optionally, expanding the population of LSK cells (e.g., HSCs) ex vivo prior to administering the LSK cells (e.g., HSCs) to the subject. In some aspects, the LSK cells (e.g., HSCs) are derived from another donor.

In some aspects, the donor subject received one or more additional agents that are capable of inducing LSK cell (e.g., HSC) mobilization into the peripheral blood. In certain aspects, the one or more additional agents comprise G-CSF, AMD3100, GM-CSF, IL-3, GM-CSF/IL-3 fusion proteins, FLK-2/FLT-3 ligand, stem cell factor, IL-6, IL-11, TPO, VEGF, plerixafor (e.g., MOZOBIL®), chemotherapy (e.g., cyclophosphamide, etoposide (e.g., TOPOSAR® and ETOPOPHOS®), POL6326 (CXCR4 antagonist), TG-0054 (CXCR4 antagonist), BKT140 (anti-SDF-1), bortezomib (proteasome inhibitor, downregulation of VLA4/VCAM-1 axis) (e.g., VELCADE®), Groβ (CXCR2 agonist, induction of MMP-9 secretion), PTH (PTH receptor agonist, expansion of BM HSC), CDX-301 (FLT3 agonist), LY2510924 (CXCR4 antagonist), natalizumab (VLA-4 antagonist) (e.g., TYSABRI®), meloxicam (Non-steroidal anti-inflammatory drug) (e.g., VIVLODEX®, MOBIC®, COMFORT®), eltrombopag (TPO receptor agonist) (e.g., PROMACTA®), ALX-0651 (Anti-CXCR4 Nanobody), or combinations thereof. In some aspects, the one or more additional agents are G-CSF, AMD3100, or both.

In some aspects, an abnormality of a hematopoietic process comprises a suppression in a bone marrow hematopoietic activity (e.g., uncontrolled differentiation of lymphoid and/or myeloid precursors). In certain aspects, the suppression of a bone marrow hematopoietic activity results in reduced number of LSK cells (e.g., HSCs) in a subject. Accordingly, in some aspects, the number of LSK cells (e.g., HSCs) in a subject having an abnormality in a hematopoietic process is decreased by at least about 10%, at least about 20%, at least about 30%, at least bout 40%, at least about 50%, at least about 60%, at least about 70%, at least about 80%, or at least about 90% or more, compared to a reference subject (e.g., a corresponding subject not suffering from an abnormality in a hematopoietic process). In some aspects, the suppression of a bone marrow hematopoietic activity results in reduced number of one or more types of blood cells in a subject. In certain aspects, the number of one or more types of blood cells in a subject having an abnormality in a hematopoietic process is decreased by at least about 10%, at least about 20%, at least about 30%, at least bout 40%, at least about 50%, at least about 60%, at least about 70%, at least about 80%, or at least about 90% or more, compared to a reference subject (e.g., a corresponding subject not suffering from an abnormality in a hematopoietic process).

In some aspects, an abnormality of a hematopoietic process that can be treated with the present disclosure is associated with a non-malignant disorder. In certain aspects, the non-malignant blood disorder comprises a myelofibrosis, myelodysplasia syndrome, amyloidosis, severe aplastic anemia, paroxysmal nocturnal hemoglobinuria, immune cytopenias, systemic sclerosis, rheumatoid arthritis, multiple sclerosis, systemic lupus erythematosus, Crohn's disease, chronic inflammatory demyelinating polyradiculoneuropathy, human immunodeficiency virus (HIV), anemia (e.g., fanconi anemia, aplastic anemia), sickle cell disease, beta thalassemia major, metabolic storage disease (e.g., Hurler's disease, Hunter's disease, or mannosidosis), adrenoleukodystrophy, metachromatic, eukodystrophy, familial erythrophagocytic lymphohistiocytosis and other histiocytic disorders, severe combined immunodeficiency (SCID), Wiskott-Aldrich syndrome, or combinations thereof.

In some aspects, an abnormality of a hematopoietic process is associated with a cancer. In certain aspects, the cancer comprises a hematological malignancy, such as acute lymphoid leukemia, acute myeloid leukemia, chronic lymphoid leukemia, chronic myeloid leukemia, diffuse large B-cell non-Hodgkin's lymphoma, mantle cell lymphoma, lymphoblastic lymphoma, Burkitt's lymphoma, follicular B-cell non-Hodgkin's lymphoma, T-cell non-Hodgkin's lymphoma, lymphocyte predominant nodular Hodgkin's lymphoma, multiple myeloma, juvenile myelomonocytic leukemia, or combinations thereof. Non-limiting examples of diseases or disorders that can be treated with the present disclosure include: mature B-cell neoplasms; mature T and NK neoplasms; Hodgkin lymphoma; posttransplant lymphoproliferative disorders (PTLD); histiocytic and dendritic cell neoplasms; myeloproliferative neoplasms (MPN); myeloid/lymphoid neoplasms with eosinophilia and rearrangement of PDGFRA, PDGFRB, or FGFR1, or with PCMI-JAK2, Myelodysplastic/myeloproliferative neoplasms (MDS/MPN), Myelodysplastic syndromes (MDS), Acute myeloid leukemia (AML) and related neoplasms, Blastic plasmacytoid dendritic cell neoplasm, Acute leukemias of ambiguous lineage, B-lymphoblastic leukemia/lymphoma, T-lymphoblastic leukemia/lymphoma, and combinations thereof. Additional disclosure relating to diseases and disorders that can be treated with the present disclosure are provided in, e.g., Swerdlow et al., Blood 127(20): 2375-2390 (2016); and Arber et al., Blood 127(20): 2391-2405 (2016); each of which is incorporated herein by reference in its entirety.

In some aspects, an abnormality of a hematopoietic process is associated with an anti-cancer therapy, wherein the anti-cancer therapy suppresses one or more bone marrow hematopoietic activity described herein. In certain aspects, the anti-cancer therapy comprises chemotherapy, radiation therapy, immunotherapy, serotherapy, targeted therapy (e.g., anti-thymocyte immunoglobulin), or combinations thereof.

In some aspects, the one or more bone marrow hematopoietic activities that are improved comprise the mobilization of LSK cells (e.g., HSCs) to the peripheral blood. Therefore, in certain aspects, after the administration of the IL-7 protein, the number of LSK cells (e.g., HSCs) present in the peripheral blood of the subject is increased by at least about 0.5-fold, 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, or at least about 100-fold or more, compared to the number of LSK cells (e.g., HSCs) present in the peripheral blood of a subject not treated with the IL-7 protein (e.g., the subject prior to the IL-7 administration or a corresponding subject treated with a different treatment regimen).

The current standard treatment for many cancers include high doses of radiation and/or chemotherapeutic drugs, which can destroy the various blood cells within a subject. In some aspects, the methods disclosed herein can be used in combination with such anti-cancer therapies to treat a cancer, wherein the present methods can help restore the LSK cells (e.g., HSCs) that are capable of differentiating into the different blood cells in the subject. Accordingly, in some aspects, a method of treating a cancer (or a tumor) in a subject in need thereof, comprises administering to the subject an effective amount of an IL-7 protein prior to the anti-cancer therapy treatment. As described herein, in certain aspects, administering an IL-7 protein disclosed herein increases the mobilization of LSK cells (e.g., HSCs) to the peripheral blood of the subject. In some aspects, the method of treating a cancer (or a tumor) further comprises isolating the mobilized LSK cells (e.g., HSCs) from the peripheral blood of the subject. In certain aspects, the mobilized LSK cells (e.g., HSCs) are transplanted back into the subject after the anti-cancer therapy treatment (e.g., autologous transplantation). In certain aspects, the LSK cells (e.g., HSCs) that are administered to a subject after an anti-cancer therapy are derived from a different donor (i.e., an individual other than the subject to be treated) (e.g., allogenic or syngenic transplantation). In such aspects, an IL-7 protein (e.g., disclosed herein) is administered to the different donor prior to isolating the LSK cells (e.g., HSCs).

In some aspects, a treatment method disclosed herein (e.g., treating an abnormality of a hematopoietic process and/or treating a cancer) comprises administering the IL-7 protein in combination with an additional agent. Non-limiting examples of additional agents that can be administered in combination with the IL-7 protein include G-CSF, CXCR4 antagonist (e.g., AMD3100, POL6326, TG-0054, LY2510924, ALX-0651), CXCR2 antagonist (e.g., bortezomib (e.g., VELCADE®), Groβ), anti-SDF-1 (e.g., BKT140), GM-CSF, IL-3, GM-CSF/IL-3 fusion proteins, FLK-2/FLT-3 ligand (e.g., CDX-301), stem cell factor, IL-6, IL-11, TPO, VEGF, VLA-4 antagonist (e.g., natalizumab (e.g., TYSABRI®)), non-steroidal anti-inflammatory drug (e.g., meloxicam (e.g., VIVLODEX®, MOBIC®, COMFORT®)), PTH receptor agonist, TPO receptor agonist (e.g., eltrombopag (e.g., PROMACTA®)), plerixafor (e.g., MOZOBIL®), chemotherapy (e.g., cyclophosphamide, etoposide (e.g., TOPOSAR® and ETOPOPHOS®), or combinations thereof.

In some aspects, the IL-7 protein (alone or in combination with an additional agent) is administered to the subject prior to the administration of the anti-cancer therapy. In some aspects, prior to the anti-cancer therapy but after the administration of the IL-7 protein (alone or in combination with an additional agent), the mobilized LSK cells (e.g., HSCs) are isolated from the peripheral blood of the subject (e.g., using any method known in the art). In certain aspects, the isolated LSK cells (e.g., HSCs) are further expanded ex vivo. In some aspects, the isolated LSK cells (e.g., HSCs) are administered to the subject after the administration of the anti-cancer therapy, wherein the administration of the isolated LSK cells (e.g., HSCs) restores the hematopoietic process within the subject.

Non-limiting examples of cancers (or tumors) that can be treated with methods disclosed herein include squamous cell carcinoma, small-cell lung cancer (SCLC), non-small cell lung cancer, squamous non-small cell lung cancer (NSCLC), nonsquamous NSCLC, gastrointestinal cancer, renal cancer (e.g., clear cell carcinoma), ovarian cancer, liver cancer (e.g., hepatocellular carcinoma), colorectal cancer, endometrial cancer, kidney cancer (e.g., renal cell carcinoma (RCC)), prostate cancer (e.g., hormone refractory prostate adenocarcinoma), thyroid cancer, pancreatic cancer, cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus (e.g., gastroesophageal junction cancer), cancer of the small intestine, cancer of the endocrine system, cancer of the parathyroid gland, cancer of the adrenal gland, sarcoma of soft tissue (e.g., rhabdomyosarcoma (RMS)), Ewing's sarcoma (or Ewing tumors), Wilms' tumor, retinoblastoma, cancer of the urethra, cancer of the penis, solid tumors of childhood, cancer of the ureter, carcinoma of the renal pelvis, tumor angiogenesis, pituitary adenoma, Kaposi's sarcoma, epidermoid cancer, squamous cell cancer, T-cell lymphoma, environmentally-induced cancers including those induced by asbestos, virus-related cancers or cancers of viral origin (e.g., human papilloma virus (HPV-related or -originating tumors)), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CIVIL), undifferentiated AML (MO), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B cell hematologic malignancy, e.g., B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukemia (T-Lbly/T-ALL), peripheral T-cell lymphoma, lymphoblastic lymphoma, post-transplantation lymphoproliferative disorder, true histiocytic lymphoma, primary effusion lymphoma, B cell lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, and any combinations thereof.

In some aspects, a cancer (or tumor) that can be treated comprises a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, carcinoma, sarcoma, leukemia, lymphoma, myeloma, germ cell tumor, or a combination thereof. In certain aspects, a cancer (or tumor) that can be treated with the present methods is breast cancer. In some aspects, breast cancer is a triple negative breast cancer (TNBC). In some aspects, a cancer (or tumor) that can be treated is a brain cancer. In certain aspects, brain cancer is a glioblastoma. In some aspects, a cancer (or tumor) that can be treated with the present methods is skin cancer. In some aspects, skin cancer is a basal cell carcinoma (BCC), cutaneous squamous cell carcinoma (cSCC), melanoma, Merkel cell carcinoma (MCC), or a combination thereof. In certain aspects, a head and neck cancer is a head and neck squamous cell carcinoma. In further aspects, a lung cancer is a small cell lung cancer (SCLC). In some aspects, an esophageal cancer is gastroesophageal junction cancer. In certain aspects, a kidney cancer is renal cell carcinoma. In some aspects, a liver cancer is hepatocellular carcinoma. In some aspects, a cancer (or tumor) that can be treated with the present disclosure is an eye cancer. In certain aspects, eye cancer comprises retinoblastoma. In some aspects, the cancer (or tumor) comprises a germ cell tumor (e.g., embryonal carcinoma, yolk sac tumor, germinoma, intracranial germ cell tumor, teratoma, and mixed germ cell tumors).

In any of the methods disclosed herein, the unit dose (e.g., for human use) of an IL-7 protein disclosed herein can be in the range of about 0.001 mg/kg to about 10 mg/kg. In certain aspects, the unit dose of an IL-7 protein is in the range of about 0.01 mg/kg to about 2 mg/kg. In some aspects, the unit dose is in the range of about 0.02 mg/kg to about 1 mg/kg. The administration of an IL-7 protein can be performed by periodic bolus injections or external reservoirs (e.g., intravenous bags) or by continuous intravenous, subcutaneous, or intraperitoneal administration from the internal (e.g., biocorrosive implants). In some aspects, an IL-7 protein is administered via subcutaneous injection. In certain aspects, an IL-7 protein disclosed herein is administered via intramuscular injection.

In some aspects, an IL-7 protein disclosed herein can be administered to a subject at a weight-based dose. In certain aspects, an IL-7 protein can be administered at a weight-based dose between about 20 μg/kg and about 600 μg/kg. In further aspects, an IL-7 protein of the present disclosure can be administered at a weight-based dose of about 20 μg/kg, about 60 μg/kg, about 120 μg/kg, about 240 μg/kg, about 360 μg/kg, about 480 μg/kg, or about 600 μg/kg. In some aspects, an IL-7 protein is administered to a subject at a dose of about 60 μg/kg.

In some aspects, an IL-7 protein disclosed herein can be administered to a subject at a dose greater than about 600 μg/kg. In certain aspects, an IL-7 protein is administered to a subject at a dose greater than about 600 μg/kg, greater than about 700 μg/kg, greater than about 800 μg/kg, greater than about 900 μg/kg, greater than about 1,000 μg/kg, greater than about 1,100 μg/kg, greater than about 1,200 μg/kg, greater than about 1,300 μg/kg, greater than about 1,400 μg/kg, greater than about 1,500 μg/kg, greater than about 1,600 μg/kg, greater than about 1,700 μg/kg, greater than about 1,800 μg/kg, greater than about 1,900 μg/kg, or greater than about 2,000 μg/kg.

In some aspects, an IL-7 protein of the present disclosure is administered at a dose of between 610 μg/kg and about 1,200 μg/kg, between 650 μg/kg and about 1,200 μg/kg, between about 700 μg/kg and about 1,200 μg/kg, between about 750 μg/kg and about 1,200 μg/kg, between about 800 μg/kg and about 1,200 μg/kg, between about 850 μg/kg and about 1,200 μg/kg, between about 900 μg/kg and about 1,200 μg/kg, between about 950 μg/kg and about 1,200 μg/kg, between about 1,000 μg/kg and about 1,200 μg/kg, between about 1,050 μg/kg and about 1,200 μg/kg, between about 1,100 μg/kg and about 1,200 μg/kg, between about 1,200 μg/kg and about 2,000 μg/kg, between about 1,300 μg/kg and about 2,000 μg/kg, between about 1,500 μg/kg and about 2,000 μg/kg, between about 1,700 μg/kg and about 2,000 μg/kg, between about 610 μg/kg and about 1,000 μg/kg, between about 650 μg/kg and about 1,000 μg/kg, between about 700 μg/kg and about 1,000 μg/kg, between about 750 μg/kg and about 1,000 μg/kg, between about 800 μg/kg and about 1,000 μg/kg, between about 850 μg/kg and about 1,000 μg/kg, between about 900 μg/kg and about 1,000 μg/kg, or between about 950 μg/kg and about 1,000 μg/kg.

In some aspects, an IL-7 protein of the present disclosure is administered at a dose of between 610 μg/kg and about 1,200 μg/kg. In certain aspects, an IL-7 protein is administered at a dose of between 650 μg/kg and about 1,200 μg/kg. In some aspects, an IL-7 protein is administered at a dose of between about 700 μg/kg and about 1,200 μg/kg. In further aspects, an IL-7 protein is administered at a dose of between about 750 μg/kg and about 1,200 μg/kg. In certain aspects, an IL-7 protein is administered at a dose of between about 800 μg/kg and about 1,200 μg/kg. In some aspects, an IL-7 protein is administered at a dose of between about 850 μg/kg and about 1,200 μg/kg. In some aspects, an IL-7 protein is administered at a dose of between about 900 μg/kg and about 1,200 μg/kg. In further aspects, an IL-7 protein is administered at a dose of between about 950 μg/kg and about 1,200 μg/kg. In some aspects, an IL-7 protein disclosed herein is administered at a dose of between about 1,000 μg/kg and about 1,200 μg/kg. In some aspects, an IL-7 protein is administered at a dose of between about 1,050 μg/kg and about 1,200 μg/kg. In some aspects, an IL-7 protein is administered at a dose of between about 1,100 μg/kg and about 1,200 μg/kg. In some aspects, an IL-7 protein is administered at a dose of between about 1,200 μg/kg and about 2,000 μg/kg. In further aspects, an IL-7 protein is administered at a dose of between about 1,300 μg/kg and about 2,000 μg/kg. In some aspects, an IL-7 protein is administered at a dose of between about 1,500 μg/kg and about 2,000 μg/kg. In some aspects, an IL-7 protein is administered at a dose of between about 1,700 μg/kg and about 2,000 μg/kg. In certain aspects, an IL-7 protein is administered at a dose of between about 610 μg/kg and about 1,000 μg/kg. In some aspects, an IL-7 protein is administered at a dose of between about 650 μg/kg and about 1,000 μg/kg. In further aspects, an IL-7 protein is administered at a dose of between about 700 μg/kg and about 1,000 μg/kg. In yet further aspects, an IL-7 protein is administered at a dose of between about 750 μg/kg and about 1,000 μg/kg. In certain aspects, an IL-7 protein is administered at a dose of between about 800 μg/kg and about 1,000 μg/kg. In some aspects, an IL-7 protein is administered at a dose of between about 850 μg/kg and about 1,000 μg/kg. In some aspects, an IL-7 protein of the present disclosure is administered at a dose of between about 900 μg/kg and about 1,000 μg/kg. In some aspects, an IL-7 protein is administered at a dose of between about 950 μg/kg and about 1,000 μg/kg.

In some aspects, an IL-7 protein is administered at a dose of between about 700 μg/kg and about 900 μg/kg, between about 750 μg/kg and about 950 μg/kg, between about 700 μg/kg and about 850 μg/kg, between about 750 μg/kg and about 850 μg/kg, between about 700 μg/kg and about 800 μg/kg, between about 800 μg/kg and about 900 μg/kg, between about 750 μg/kg and about 850 μg/kg, or between about 850 μg/kg and about 950 μg/kg. In some aspects, an IL-7 protein is administered at a dose of between about 700 μg/kg and about 900 μg/kg. In certain aspects, an IL-7 protein is administered at a dose of between about 750 μg/kg and about 950 μg/kg. In further aspects, an IL-7 protein is administered at a dose of between about 700 μg/kg and about 850 μg/kg. In some aspects, an IL-7 protein is administered at a dose of between about 750 μg/kg and about 850 μg/kg. In other aspects, an IL-7 protein is administered at a dose of between about 700 μg/kg and about 800 μg/kg. In some aspects, an IL-7 protein is administered at a dose of between about 800 μg/kg and about 900 μg/kg. In some aspects, an IL-7 protein is administered at a dose of between about 750 μg/kg and about 850 μg/kg. In certain aspects, an IL-7 protein is administered at a dose of between about 850 μg/kg and about 950 μg/kg.

In some aspects, an IL-7 protein is administered at a dose of about 650 μg/kg, about 680 μg/kg, about 700 μg/kg, about 720 μg/kg, about 740 μg/kg, about 750 μg/kg, about 760 μg/kg, about 780 μg/kg, about 800 μg/kg, about 820 μg/kg, about 840 μg/kg, about 850 μg/kg, about 860 μg/kg, about 880 μg/kg, about 900 μg/kg, about 920 μg/kg, about 940 μg/kg, about 950 μg/kg, about 960 μg/kg, about 980 μg/kg, about 1,000 μg/kg, about 1,020 μg/kg, about 1,020 μg/kg, about 1,040 μg/kg, about 1,060 μg/kg, about 1,080 μg/kg, about 1,100 μg/kg, about 1,120 μg/kg, about 1,140 μg/kg, about 1,160 μg/kg, about 1,180 μg/kg, about 1200 μg/kg, about 1,220 μg/kg, about 1,240 μg/kg, about 1,260 μg/kg, about 1,280 μg/kg, about 1,300 μg/kg, about 1,320 μg/kg, about 1,340 μg/kg, about 1,360 μg/kg, about 1,380 μg/kg, about 1,400 μg/kg, about 1,420 μg/kg, about 1,440 μg/kg, about 1,460 μg/kg, about 1,480 μg/kg, about 1,500 μg/kg, about 1,520 μg/kg, about 1,540 μg/kg, about 1,560 μg/kg, about 1,580 μg/kg, about 1,600 μg/kg, about 1,620 μg/kg, about 1,640 μg/kg, about 1,660 μg/kg, about 1,680 μg/kg, about 1,700 μg/kg, about 1,720 μg/kg, about 1,740 μg/kg, about 1,760 μg/kg, about 1,780 μg/kg, about 1,800 μg/kg, about 1,820 μg/kg, about 1,840 μg/kg, about 1,860 μg/kg, about 1,880 μg/kg, about 1,900 μg/kg, about 1,920 μg/kg, about 1,940 μg/kg, about 1,960 μg/kg, about 1,980 μg/kg, or about 2,000 μg/kg.

In some aspects, an IL-7 protein is administered at a dose of about 650 μg/kg. In other aspects, an IL-7 protein disclosed herein is administered at a dose of about 680 μg/kg. In some aspects, an IL-7 protein is administered at a dose of about 700 μg/kg. In some aspects, an IL-7 protein is administered at a dose of about 720 μg/kg. In certain aspects, an IL-7 protein is administered at a dose of about 740 μg/kg. In some aspects, an IL-7 protein is administered at a dose of about 750 μg/kg. In some aspects, an IL-7 protein is administered at a dose of about 760 μg/kg. In certain aspects, an IL-7 protein is administered at a dose of about 780 μg/kg. In some aspects, an IL-7 protein is administered at a dose of about 800 μg/kg. In further aspects, an IL-7 protein is administered at a dose of about 820 μg/kg. In certain aspects, an IL-7 protein is administered at a dose of about 840 μg/kg. In some aspects, an IL-7 protein is administered at a dose of about 850 μg/kg. In certain aspects, an IL-7 protein is administered at a dose of about 860 μg/kg. In some aspects, an IL-7 protein is administered at a dose of about 880 μg/kg. In some aspects, an IL-7 protein is administered at a dose of about 900 μg/kg. In further aspects, an IL-7 protein is administered at a dose of about 920 μg/kg. In some aspects, an IL-7 protein is administered at a dose of about 940 μg/kg. In further aspects, an IL-7 protein is administered at a dose of about 950 μg/kg. In some aspects, an IL-7 protein is administered at a dose of about 960 μg/kg. In certain aspects, an IL-7 protein is administered at a dose of about 980 μg/kg. In some aspects, an IL-7 protein is administered at a dose of about 1,000 μg/kg. In certain aspects, an IL-7 protein is administered at a dose of about 1,020 μg/kg. In further aspects, an IL-7 protein is administered at a dose of about 1,040 μg/kg. In some aspects, an IL-7 protein is administered at a dose of about 1,060 μg/kg. In other aspects, an IL-7 protein is administered at a dose of about 1,080 μg/kg. In some aspects, an IL-7 protein is administered at a dose of about 1,100 μg/kg. In certain aspects, an IL-7 protein is administered at a dose of about 1,120 μg/kg. In further aspects, an IL-7 protein is administered at a dose of about 1,140 μg/kg. In some aspects, an IL-7 protein is administered at a dose of about 1,160 μg/kg. In other aspects, an IL-7 protein is administered at a dose of about 1,180 μg/kg. In certain aspects, an IL-7 protein is administered at a dose of about 1,200 μg/kg. In certain aspects, an IL-7 protein is administered at a dose of about 1,220 μg/kg. In further aspects, an IL-7 protein is administered at a dose of about 1,240 μg/kg. In some aspects, an IL-7 protein is administered at a dose of about 1,260 μg/kg. In other aspects, an IL-7 protein is administered at a dose of about 1,280 μg/kg. In some aspects, an IL-7 protein is administered at a dose of about 1,300 μg/kg. In certain aspects, an IL-7 protein is administered at a dose of about 1,320 μg/kg. In further aspects, an IL-7 protein is administered at a dose of about 1,340 μg/kg. In some aspects, an IL-7 protein is administered at a dose of about 1,360 μg/kg. In other aspects, an IL-7 protein is administered at a dose of about 1,380 μg/kg. In further aspects, an IL-7 protein is administered at a dose of about 1,400 μg/kg. In certain aspects, an IL-7 protein is administered at a dose of about 1,420 μg/kg. In some aspects, an IL-7 protein is administered at a dose of about 1,440 μg/kg. In some aspects, an IL-7 protein is administered at a dose of about 1,460 μg/kg. In other aspects, an IL-7 protein is administered at a dose of about 1,480 μg/kg. In certain aspects, an IL-7 protein is administered at a dose of about 1,500 μg/kg. In certain aspects, an IL-7 protein is administered at a dose of about 1,520 μg/kg. In further aspects, an IL-7 protein is administered at a dose of about 1,540 μg/kg. In some aspects, an IL-7 protein is administered at a dose of about 1,560 μg/kg. In other aspects, an IL-7 protein is administered at a dose of about 1,580 μg/kg. In some aspects, an IL-7 protein is administered at a dose of about 1,600 μg/kg. In certain aspects, an IL-7 protein is administered at a dose of about 1,620 μg/kg. In further aspects, an IL-7 protein is administered at a dose of about 1,640 μg/kg. In some aspects, an IL-7 protein is administered at a dose of about 1,660 μg/kg. In other aspects, an IL-7 protein is administered at a dose of about 1,680 μg/kg. In certain aspects, an IL-7 protein is administered at a dose of about 1,700 μg/kg. In certain aspects, an IL-7 protein is administered at a dose of about 1,720 μg/kg. In further aspects, an IL-7 protein is administered at a dose of about 1,740 μg/kg. In some aspects, an IL-7 protein is administered at a dose of about 1,760 μg/kg. In other aspects, an IL-7 protein is administered at a dose of about 1,780 μg/kg. In some aspects, an IL-7 protein is administered at a dose of about 1,800 μg/kg. In certain aspects, an IL-7 protein is administered at a dose of about 1,820 μg/kg. In further aspects, an IL-7 protein is administered at a dose of about 1,840 μg/kg. In some aspects, an IL-7 protein is administered at a dose of about 1,860 μg/kg. In other aspects, an IL-7 protein is administered at a dose of about 1,880 μg/kg. In some aspects, an IL-7 protein is administered at a dose of about 1,900 μg/kg. In certain aspects, an IL-7 protein is administered at a dose of about 1,920 μg/kg. In further aspects, an IL-7 protein is administered at a dose of about 1,940 μg/kg. In some aspects, an IL-7 protein is administered at a dose of about 1,960 μg/kg. In other aspects, an IL-7 protein is administered at a dose of about 1,980 μg/kg. In further aspects, an IL-7 protein is administered at a dose of about 2,000 μg/kg.

In some aspects, an IL-7 protein can be administered at a flat dose. In certain aspects, an IL-7 protein can be administered at a flat dose of about 0.25 mg to about 9 mg. In some aspects, an IL-7 protein can be administered at a flat dose of about 0.25 mg, about 1 mg, about 3 mg, about 6 mg, or about 9 mg.

In some aspects, an IL-7 protein disclosed herein is administered to a subject at multiple doses (i.e., repeated administrations). In certain embodiments, an IL-7 protein is administered to the subject at least two times, at least three times, at least four times, at least five times, at least six times, at least seven times, at least eight times, at least nine times, or at least ten times or more. In other embodiments, a subject receives a single administration of the IL-7 protein (e.g., prior to, concurrently, or after an administration of an immune checkpoint inhibitor).

In some aspects, an IL-7 protein is administered at a dosing frequency of about once a week, about once in two weeks, about once in three weeks, about once in four weeks, about once in five weeks, about once in six weeks, about once in seven weeks, about once in eight weeks, about once in nine weeks, about once in 10 weeks, about once in 11 weeks, or about once in 12 weeks. In certain aspects, an IL-7 protein is administered at a dosing frequency of about once every 10 days, about once every 20 days, about once every 30 days, about once every 40 days, about once every 50 days, about once every 60 days, about once every 70 days, about once every 80 days, about once every 90 days, or about once every 100 days. In some aspects, the IL-7 protein is administered once in three weeks. In some aspects, the IL-7 protein is administered once a week. In some aspects, the IL-7 protein is administered once in two weeks. In certain aspects, the IL-7 protein is administered once in three weeks. In some aspects, the IL-7 protein is administered once in four weeks. In certain aspects, the IL-7 protein is administered once in six weeks. In further aspects, the IL-7 protein is administered once in eight weeks. In some aspects, the IL-7 protein is administered once in nine weeks. In certain aspects, the IL-7 protein is administered once in 12 weeks. In some aspects, the IL-7 protein is administered once every 10 days. In certain aspects, the IL-7 protein is administered once every 20 days. In other aspects, the IL-7 protein is administered once every 30 days. In some aspects, the IL-7 protein is administered once every 40 days. In certain aspects, the IL-7 protein is administered once every 50 days. In some aspects, the IL-7 protein is administered once every 60 days. In further aspects, the IL-7 protein is administered once every 70 days. In some aspects, the IL-7 protein is administered once every 80 days. In certain aspects, the IL-7 protein is administered once every days. In some aspects, the IL-7 protein is administered once every 100 days.

In some aspects, the IL-7 protein is administered twice or more times in an amount of about 720 μg/kg at an interval of about 1 week, about 2 weeks, about 3 weeks, or about 4 weeks. In some aspects, the IL-7 protein is administered twice or more times in an amount of about 840 μg/kg at an interval of about 2 weeks, about 3 weeks, about 4 weeks, or about 5 weeks. In some aspects, the IL-7 protein is administered twice or more times in an amount of about 960 μg/kg at an interval of about 2 weeks, about 3 weeks, about 4 weeks, about 5 weeks, or about 6 weeks. In some aspects, the IL-7 protein is administered twice or more times in an amount of about 1200 μg/kg at an interval of about 3 weeks, about 4 weeks, about 5 weeks, about 6 weeks, about 7 weeks, or about 8 weeks. In some aspects, the IL-7 protein is administered twice or more times in an amount of about 1440 μg/kg at an interval of about 3 weeks, about 4 weeks, about 5 weeks, about 6 weeks, about 7 weeks, about 8 weeks, about 2 months, about 8 weeks, about 10 weeks, about 12 weeks, or about 3 months.

In some aspects, the IL-7 protein is administered at a dose of 60 μg/kg with a dosing frequency of once a week. In some aspects, the IL-7 protein is administered at a dose of 120 μg/kg with a dosing frequency of once a week. In some aspects, the IL-7 protein is administered at a dose of 240 μg/kg with a dosing frequency of once a week. In some aspects, the IL-7 protein is administered at a dose of 480 μg/kg with a dosing frequency of once a week. In some aspects, the IL-7 protein is administered at a dose of 720 μg/kg with a dosing frequency of once a week. In some aspects, the IL-7 protein is administered at a dose of 960 μg/kg with a dosing frequency of once a week. In some aspects, the IL-7 protein is administered at a dose of 1,200 μg/kg with a dosing frequency of once a week. In some aspects, the IL-7 protein is administered at a dose of 1,300 μg/kg with a dosing frequency of once a week. In some aspects, the IL-7 protein is administered at a dose of 1,400 μg/kg with a dosing frequency of once a week. In other aspects, the IL-7 protein is administered at a dose of 1,420 μg/kg with a dosing frequency of once a week. In certain aspects, the IL-7 protein is administered at a dose of 1,440 μg/kg with a dosing frequency of once a week. In further aspects, the IL-7 protein is administered at a dose of 1,460 μg/kg with a dosing frequency of once a week. In certain aspects, the IL-7 protein is administered at a dose of 1,480 μg/kg with a dosing frequency of once a week. In some aspects, the IL-7 protein is administered at a dose of 1,500 μg/kg with a dosing frequency of once a week. In further aspects, the IL-7 protein is administered at a dose of 1,600 μg/kg with a dosing frequency of once a week. In some aspects, the IL-7 protein is administered at a dose of 1,700 μg/kg with a dosing frequency of once a week. In some aspects, the IL-7 protein is administered at a dose of 2,000 μg/kg with a dosing frequency of once a week.

In some aspects, the IL-7 protein is administered at a dose of 60 μg/kg with a dosing frequency of once in two weeks. In some aspects, the IL-7 protein is administered at a dose of 120 μg/kg with a dosing frequency of once in two weeks. In some aspects, the IL-7 protein is administered at a dose of 240 μg/kg with a dosing frequency of once in two weeks. In some aspects, the IL-7 protein is administered at a dose of 480 μg/kg with a dosing frequency of once in two weeks. In some aspects, the IL-7 protein is administered at a dose of 720 μg/kg with a dosing frequency of once in two weeks. In some aspects, the IL-7 protein is administered at a dose of 960 μg/kg with a dosing frequency of once in two weeks. In some aspects, the IL-7 protein is administered at a dose of 1,200 μg/kg with a dosing frequency of once in two weeks. In some aspects, the IL-7 protein is administered at a dose of 1,300 μg/kg with a dosing frequency of once in two weeks. In some aspects, the IL-7 protein is administered at a dose of 1,400 μg/kg with a dosing frequency of once in two weeks. In other aspects, the IL-7 protein is administered at a dose of 1,420 μg/kg with a dosing frequency of once in two weeks. In certain aspects, the IL-7 protein is administered at a dose of 1,440 μg/kg with a dosing frequency of once in two weeks. In further aspects, the IL-7 protein is administered at a dose of 1,460 μg/kg with a dosing frequency of once in two weeks. In certain aspects, the IL-7 protein is administered at a dose of 1,480 μg/kg with a dosing frequency of once in two weeks. In some aspects, the IL-7 protein is administered at a dose of 1,500 μg/kg with a dosing frequency of once in two weeks. In further aspects, the IL-7 protein is administered at a dose of 1,600 μg/kg with a dosing frequency of once in two weeks. In some aspects, the IL-7 protein is administered at a dose of 1,700 μg/kg with a dosing frequency of once in two weeks. In some aspects, the IL-7 protein is administered at a dose of 2,000 μg/kg with a dosing frequency of once in two weeks.

In some aspects, the IL-7 protein is administered at a dose of 60 μg/kg with a dosing frequency of once in three weeks. In some aspects, the IL-7 protein is administered at a dose of 120 μg/kg with a dosing frequency of once in three weeks. In some aspects, the IL-7 protein is administered at a dose of 240 μg/kg with a dosing frequency of once in three weeks. In some aspects, the IL-7 protein is administered at a dose of 480 μg/kg with a dosing frequency of once in three weeks. In some aspects, the IL-7 protein is administered at a dose of 720 μg/kg with a dosing frequency of once in three weeks. In some aspects, the IL-7 protein is administered at a dose of 960 μg/kg with a dosing frequency of once in three weeks. In some aspects, the IL-7 protein is administered at a dose of 1,200 μg/kg with a dosing frequency of once in three weeks. In some aspects, the IL-7 protein is administered at a dose of 1,300 μg/kg with a dosing frequency of once in three weeks. In some aspects, the IL-7 protein is administered at a dose of 1,400 μg/kg with a dosing frequency of once in three weeks. In other aspects, the IL-7 protein is administered at a dose of 1,420 μg/kg with a dosing frequency of once in three weeks. In certain aspects, the IL-7 protein is administered at a dose of 1,440 μg/kg with a dosing frequency of once in three weeks. In further aspects, the IL-7 protein is administered at a dose of 1,460 μg/kg with a dosing frequency of once in three weeks. In certain aspects, the IL-7 protein is administered at a dose of 1,480 μg/kg with a dosing frequency of once in three weeks. In some aspects, the IL-7 protein is administered at a dose of 1,500 μg/kg with a dosing frequency of once in three weeks. In further aspects, the IL-7 protein is administered at a dose of 1,600 μg/kg with a dosing frequency of once in three weeks. In some aspects, the IL-7 protein is administered at a dose of 1,700 μg/kg with a dosing frequency of once in three weeks. In some aspects, the IL-7 protein is administered at a dose of 2,000 μg/kg with a dosing frequency of once in three weeks.

In some aspects, the IL-7 protein is administered at a dose of 60 μg/kg with a dosing frequency of once in four weeks. In some aspects, the IL-7 protein is administered at a dose of 120 μg/kg with a dosing frequency of once in four weeks. In some aspects, the IL-7 protein is administered at a dose of 240 μg/kg with a dosing frequency of once in four weeks. In some aspects, the IL-7 protein is administered at a dose of 480 μg/kg with a dosing frequency of once in four weeks. In some aspects, the IL-7 protein is administered at a dose of 720 μg/kg with a dosing frequency of once in four weeks. In some aspects, the IL-7 protein is administered at a dose of 960 μg/kg with a dosing frequency of once in four weeks. In some aspects, the IL-7 protein is administered at a dose of 1,200 μg/kg with a dosing frequency of once in four weeks. In some aspects, the IL-7 protein is administered at a dose of 1,300 μg/kg with a dosing frequency of once in four weeks. In some aspects, the IL-7 protein is administered at a dose of 1,400 μg/kg with a dosing frequency of once in four weeks. In other aspects, the IL-7 protein is administered at a dose of 1,420 μg/kg with a dosing frequency of once in four weeks. In certain aspects, the IL-7 protein is administered at a dose of 1,440 μg/kg with a dosing frequency of once in four weeks. In further aspects, the IL-7 protein is administered at a dose of 1,460 μg/kg with a dosing frequency of once in four weeks. In certain aspects, the IL-7 protein is administered at a dose of 1,480 μg/kg with a dosing frequency of once in four weeks. In some aspects, the IL-7 protein is administered at a dose of 1,500 μg/kg with a dosing frequency of once in four weeks. In further aspects, the IL-7 protein is administered at a dose of 1,600 μg/kg with a dosing frequency of once in four weeks. In some aspects, the IL-7 protein is administered at a dose of 1,700 μg/kg with a dosing frequency of once in four weeks. In some aspects, the IL-7 protein is administered at a dose of 2,000 μg/kg with a dosing frequency of once in four weeks.

In some aspects, the IL-7 protein is administered at a dose of 60 μg/kg with a dosing frequency of once in five weeks. In some aspects, the IL-7 protein is administered at a dose of 120 μg/kg with a dosing frequency of once in five weeks. In some aspects, the IL-7 protein is administered at a dose of 240 μg/kg with a dosing frequency of once in five weeks. In some aspects, the IL-7 protein is administered at a dose of 480 μg/kg with a dosing frequency of once in five weeks. In some aspects, the IL-7 protein is administered at a dose of 720 μg/kg with a dosing frequency of once in five weeks. In some aspects, the IL-7 protein is administered at a dose of 960 μg/kg with a dosing frequency of once in five weeks. In some aspects, the IL-7 protein is administered at a dose of 1,200 μg/kg with a dosing frequency of once in five weeks. In some aspects, the IL-7 protein is administered at a dose of 1,300 μg/kg with a dosing frequency of once in five weeks. In some aspects, the IL-7 protein is administered at a dose of 1,400 μg/kg with a dosing frequency of once in five weeks. In other aspects, the IL-7 protein is administered at a dose of 1,420 μg/kg with a dosing frequency of once in five weeks. In certain aspects, the IL-7 protein is administered at a dose of 1,440 μg/kg with a dosing frequency of once in five weeks. In further aspects, the IL-7 protein is administered at a dose of 1,460 μg/kg with a dosing frequency of once in five weeks. In certain aspects, the IL-7 protein is administered at a dose of 1,480 μg/kg with a dosing frequency of once in five weeks. In some aspects, the IL-7 protein is administered at a dose of 1,500 μg/kg with a dosing frequency of once in five weeks. In further aspects, the IL-7 protein is administered at a dose of 1,600 μg/kg with a dosing frequency of once in five weeks. In some aspects, the IL-7 protein is administered at a dose of 1,700 μg/kg with a dosing frequency of once in five weeks. In some aspects, the IL-7 protein is administered at a dose of 2,000 μg/kg with a dosing frequency of once in five weeks.

In some aspects, the IL-7 protein is administered at a dose of 60 μg/kg with a dosing frequency of once in six weeks. In some aspects, the IL-7 protein is administered at a dose of 120 μg/kg with a dosing frequency of once in six weeks. In some aspects, the IL-7 protein is administered at a dose of 240 μg/kg with a dosing frequency of once in six weeks. In some aspects, the IL-7 protein is administered at a dose of 480 μg/kg with a dosing frequency of once in six weeks. In some aspects, the IL-7 protein is administered at a dose of 720 μg/kg with a dosing frequency of once in six weeks. In some aspects, the IL-7 protein is administered at a dose of 960 μg/kg with a dosing frequency of once in six weeks. In some aspects, the IL-7 protein is administered at a dose of 1,200 μg/kg with a dosing frequency of once in six weeks. In some aspects, the IL-7 protein is administered at a dose of 1,300 μg/kg with a dosing frequency of once in six weeks. In some aspects, the IL-7 protein is administered at a dose of 1,400 μg/kg with a dosing frequency of once in six weeks. In other aspects, the IL-7 protein is administered at a dose of 1,420 μg/kg with a dosing frequency of once in six weeks. In certain aspects, the IL-7 protein is administered at a dose of 1,440 μg/kg with a dosing frequency of once in six weeks. In further aspects, the IL-7 protein is administered at a dose of 1,460 μg/kg with a dosing frequency of once in six weeks. In certain aspects, the IL-7 protein is administered at a dose of 1,480 μg/kg with a dosing frequency of once in six weeks. In some aspects, the IL-7 protein is administered at a dose of 1,500 μg/kg with a dosing frequency of once in six weeks. In further aspects, the IL-7 protein is administered at a dose of 1,600 μg/kg with a dosing frequency of once in six weeks. In some aspects, the IL-7 protein is administered at a dose of 1,700 μg/kg with a dosing frequency of once in six weeks. In some aspects, the IL-7 protein is administered at a dose of 2,000 μg/kg with a dosing frequency of once in six weeks.

In some aspects, the IL-7 protein is administered at a dose of 60 μg/kg with a dosing frequency of once in seven weeks. In some aspects, the IL-7 protein is administered at a dose of 120 μg/kg with a dosing frequency of once in seven weeks. In some aspects, the IL-7 protein is administered at a dose of 240 μg/kg with a dosing frequency of once in seven weeks. In some aspects, the IL-7 protein is administered at a dose of 480 μg/kg with a dosing frequency of once in seven weeks. In some aspects, the IL-7 protein is administered at a dose of 720 μg/kg with a dosing frequency of once in seven weeks. In some aspects, the IL-7 protein is administered at a dose of 960 μg/kg with a dosing frequency of once in seven weeks. In some aspects, the IL-7 protein is administered at a dose of 1,200 μg/kg with a dosing frequency of once in seven weeks. In some aspects, the IL-7 protein is administered at a dose of 1,300 μg/kg with a dosing frequency of once in seven weeks. In some aspects, the IL-7 protein is administered at a dose of 1,400 μg/kg with a dosing frequency of once in seven weeks. In other aspects, the IL-7 protein is administered at a dose of 1,420 μg/kg with a dosing frequency of once in seven weeks. In certain aspects, the IL-7 protein is administered at a dose of 1,440 μg/kg with a dosing frequency of once in seven weeks. In further aspects, the IL-7 protein is administered at a dose of 1,460 μg/kg with a dosing frequency of once in seven weeks. In certain aspects, the IL-7 protein is administered at a dose of 1,480 μg/kg with a dosing frequency of once in seven weeks. In some aspects, the IL-7 protein is administered at a dose of 1,500 μg/kg with a dosing frequency of once in seven weeks. In further aspects, the IL-7 protein is administered at a dose of 1,600 μg/kg with a dosing frequency of once in seven weeks. In some aspects, the IL-7 protein is administered at a dose of 1,700 μg/kg with a dosing frequency of once in seven weeks. In some aspects, the IL-7 protein is administered at a dose of 2,000 μg/kg with a dosing frequency of once in seven weeks.

In some aspects, the IL-7 protein is administered at a dose of 60 μg/kg with a dosing frequency of once in eight weeks. In some aspects, the IL-7 protein is administered at a dose of 120 μg/kg with a dosing frequency of once in eight weeks. In some aspects, the IL-7 protein is administered at a dose of 240 μg/kg with a dosing frequency of once in eight weeks. In some aspects, the IL-7 protein is administered at a dose of 480 μg/kg with a dosing frequency of once in eight weeks. In some aspects, the IL-7 protein is administered at a dose of 720 μg/kg with a dosing frequency of once in eight weeks. In some aspects, the IL-7 protein is administered at a dose of 960 μg/kg with a dosing frequency of once in eight weeks. In some aspects, the IL-7 protein is administered at a dose of 1,200 μg/kg with a dosing frequency of once in eight weeks. In some aspects, the IL-7 protein is administered at a dose of 1,300 μg/kg with a dosing frequency of once in eight weeks. In some aspects, the IL-7 protein is administered at a dose of 1,400 μg/kg with a dosing frequency of once in eight weeks. In other aspects, the IL-7 protein is administered at a dose of 1,420 μg/kg with a dosing frequency of once in eight weeks. In certain aspects, the IL-7 protein is administered at a dose of 1,440 μg/kg with a dosing frequency of once in eight weeks. In further aspects, the IL-7 protein is administered at a dose of 1,460 μg/kg with a dosing frequency of once in eight weeks. In certain aspects, the IL-7 protein is administered at a dose of 1,480 μg/kg with a dosing frequency of once in eight weeks. In some aspects, the IL-7 protein is administered at a dose of 1,500 μg/kg with a dosing frequency of once in eight weeks. In further aspects, the IL-7 protein is administered at a dose of 1,600 μg/kg with a dosing frequency of once in eight weeks. In some aspects, the IL-7 protein is administered at a dose of 1,700 μg/kg with a dosing frequency of once in eight weeks. In some aspects, the IL-7 protein is administered at a dose of 2,000 μg/kg with a dosing frequency of once in eight weeks.

In some aspects, the IL-7 protein is administered at a dose of 60 μg/kg with a dosing frequency of once in nine weeks. In some aspects, the IL-7 protein is administered at a dose of 120 μg/kg with a dosing frequency of once in nine weeks. In some aspects, the IL-7 protein is administered at a dose of 240 μg/kg with a dosing frequency of once in nine weeks. In some aspects, the IL-7 protein is administered at a dose of 480 μg/kg with a dosing frequency of once in nine weeks. In some aspects, the IL-7 protein is administered at a dose of 720 μg/kg with a dosing frequency of once in nine weeks. In some aspects, the IL-7 protein is administered at a dose of 960 μg/kg with a dosing frequency of once in nine weeks. In some aspects, the IL-7 protein is administered at a dose of 1,200 μg/kg with a dosing frequency of once in nine weeks. In some aspects, the IL-7 protein is administered at a dose of 1,300 μg/kg with a dosing frequency of once in three weeks. In some aspects, the IL-7 protein is administered at a dose of 1,400 μg/kg with a dosing frequency of once in three weeks. In other aspects, the IL-7 protein is administered at a dose of 1,420 μg/kg with a dosing frequency of once in three weeks. In certain aspects, the IL-7 protein is administered at a dose of 1,440 μg/kg with a dosing frequency of once in nine weeks. In further aspects, the IL-7 protein is administered at a dose of 1,460 μg/kg with a dosing frequency of once in three weeks. In certain aspects, the IL-7 protein is administered at a dose of 1,480 μg/kg with a dosing frequency of once in three weeks. In some aspects, the IL-7 protein is administered at a dose of 1,500 μg/kg with a dosing frequency of once in three weeks. In further aspects, the IL-7 protein is administered at a dose of 1,600 μg/kg with a dosing frequency of once in three weeks. In some aspects, the IL-7 protein is administered at a dose of 1,700 μg/kg with a dosing frequency of once in nine weeks. In some aspects, the IL-7 protein is administered at a dose of 2,000 μg/kg with a dosing frequency of once in nine weeks.

In some aspects, the IL-7 protein is administered at a dose of 60 μg/kg with a dosing frequency of once in 10 weeks. In some aspects, the IL-7 protein is administered at a dose of 120 μg/kg with a dosing frequency of once in 10 weeks. In some aspects, the IL-7 protein is administered at a dose of 240 μg/kg with a dosing frequency of once in 10 weeks. In some aspects, the IL-7 protein is administered at a dose of 480 μg/kg with a dosing frequency of once in 10 weeks. In some aspects, the IL-7 protein is administered at a dose of 720 μg/kg with a dosing frequency of once in 10 weeks. In some aspects, the IL-7 protein is administered at a dose of 960 μg/kg with a dosing frequency of once in 10 weeks. In some aspects, the IL-7 protein is administered at a dose of 1,200 μg/kg with a dosing frequency of once in 10 weeks. In some aspects, the IL-7 protein is administered at a dose of 1,300 μg/kg with a dosing frequency of once in 10 weeks. In some aspects, the IL-7 protein is administered at a dose of 1,400 μg/kg with a dosing frequency of once in 10 weeks. In other aspects, the IL-7 protein is administered at a dose of 1,420 μg/kg with a dosing frequency of once in 10 weeks. In certain aspects, the IL-7 protein is administered at a dose of 1,440 μg/kg with a dosing frequency of once in 10 weeks. In further aspects, the IL-7 protein is administered at a dose of 1,460 μg/kg with a dosing frequency of once in 10 weeks. In certain aspects, the IL-7 protein is administered at a dose of 1,480 μg/kg with a dosing frequency of once in 10 weeks. In some aspects, the IL-7 protein is administered at a dose of 1,500 μg/kg with a dosing frequency of once in 10 weeks. In further aspects, the IL-7 protein is administered at a dose of 1,600 μg/kg with a dosing frequency of once in 10 weeks. In some aspects, the IL-7 protein is administered at a dose of 1,700 μg/kg with a dosing frequency of once in 10 weeks. In some aspects, the IL-7 protein is administered at a dose of 2,000 μg/kg with a dosing frequency of once in 10 weeks.

In some aspects, the IL-7 protein is administered at a dose of 60 μg/kg with a dosing frequency of once in 11 weeks. In some aspects, the IL-7 protein is administered at a dose of 120 μg/kg with a dosing frequency of once in 11 weeks. In some aspects, the IL-7 protein is administered at a dose of 240 μg/kg with a dosing frequency of once in 11 weeks. In some aspects, the IL-7 protein is administered at a dose of 480 μg/kg with a dosing frequency of once in 11 weeks. In some aspects, the IL-7 protein is administered at a dose of 720 μg/kg with a dosing frequency of once in 11 weeks. In some aspects, the IL-7 protein is administered at a dose of 960 μg/kg with a dosing frequency of once in 11 weeks. In some aspects, the IL-7 protein is administered at a dose of 1,200 μg/kg with a dosing frequency of once in 11 weeks. In some aspects, the IL-7 protein is administered at a dose of 1,300 μg/kg with a dosing frequency of once in 11 weeks. In some aspects, the IL-7 protein is administered at a dose of 1,400 μg/kg with a dosing frequency of once in 11 weeks. In other aspects, the IL-7 protein is administered at a dose of 1,420 μg/kg with a dosing frequency of once in 11 weeks. In certain aspects, the IL-7 protein is administered at a dose of 1,440 μg/kg with a dosing frequency of once in 11 weeks. In further aspects, the IL-7 protein is administered at a dose of 1,460 μg/kg with a dosing frequency of once in 11 weeks. In certain aspects, the IL-7 protein is administered at a dose of 1,480 μg/kg with a dosing frequency of once in 11 weeks. In some aspects, the IL-7 protein is administered at a dose of 1,500 μg/kg with a dosing frequency of once in 11 weeks. In further aspects, the IL-7 protein is administered at a dose of 1,600 μg/kg with a dosing frequency of once in 11 weeks. In some aspects, the IL-7 protein is administered at a dose of 1,700 μg/kg with a dosing frequency of once in 11 weeks. In some aspects, the IL-7 protein is administered at a dose of 2,000 μg/kg with a dosing frequency of once in 11 weeks.

In some aspects, the IL-7 protein is administered at a dose of 60 μg/kg with a dosing frequency of once in 12 weeks. In some aspects, the IL-7 protein is administered at a dose of 120 μg/kg with a dosing frequency of once in 12 weeks. In some aspects, the IL-7 protein is administered at a dose of 240 μg/kg with a dosing frequency of once in 12 weeks. In some aspects, the IL-7 protein is administered at a dose of 480 μg/kg with a dosing frequency of once in 12 weeks. In some aspects, the IL-7 protein is administered at a dose of 720 μg/kg with a dosing frequency of once in 12 weeks. In some aspects, the IL-7 protein is administered at a dose of 960 μg/kg with a dosing frequency of once in 12 weeks. In some aspects, the IL-7 protein is administered at a dose of 1,200 μg/kg with a dosing frequency of once in 12 weeks. In some aspects, the IL-7 protein is administered at a dose of 1,300 μg/kg with a dosing frequency of once in 12 weeks. In some aspects, the IL-7 protein is administered at a dose of 1,400 μg/kg with a dosing frequency of once in 12 weeks. In other aspects, the IL-7 protein is administered at a dose of 1,420 μg/kg with a dosing frequency of once in 12 weeks. In certain aspects, the IL-7 protein is administered at a dose of 1,440 μg/kg with a dosing frequency of once in 12 weeks. In further aspects, the IL-7 protein is administered at a dose of 1,460 μg/kg with a dosing frequency of once in 12 weeks. In certain aspects, the IL-7 protein is administered at a dose of 1,480 μg/kg with a dosing frequency of once in 12 weeks. In some aspects, the IL-7 protein is administered at a dose of 1,500 μg/kg with a dosing frequency of once in 12 weeks. In further aspects, the IL-7 protein is administered at a dose of 1,600 μg/kg with a dosing frequency of once in 12 weeks. In some aspects, the IL-7 protein is administered at a dose of 1,700 μg/kg with a dosing frequency of once in 12 weeks. In some aspects, the IL-7 protein is administered at a dose of 2,000 μg/kg with a dosing frequency of once in 12 weeks.

In some aspects, the IL-7 protein is administered at a dose of 60 μg/kg with a dosing frequency of once every 10 days. In some aspects, the IL-7 protein is administered at a dose of 120 μg/kg with a dosing frequency of once every 10 days. In some aspects, the IL-7 protein is administered at a dose of 240 μg/kg with a dosing frequency of once every 10 days. In some aspects, the IL-7 protein is administered at a dose of 480 μg/kg with a dosing frequency of once every 10 days. In some aspects, the IL-7 protein is administered at a dose of 720 μg/kg with a dosing frequency of once every 10 days. In some aspects, the IL-7 protein is administered at a dose of 960 μg/kg with a dosing frequency of once every 10 days. In some aspects, the IL-7 protein is administered at a dose of 1,200 μg/kg with a dosing frequency of once every 10 days. In some aspects, the IL-7 protein is administered at a dose of 1,300 μg/kg with a dosing frequency of once every 10 days. In some aspects, the IL-7 protein is administered at a dose of 1,400 μg/kg with a dosing frequency of once every 10 days. In other aspects, the IL-7 protein is administered at a dose of 1,420 μg/kg with a dosing frequency of once every 10 days. In certain aspects, the IL-7 protein is administered at a dose of 1,440 μg/kg with a dosing frequency of once every 10 days. In further aspects, the IL-7 protein is administered at a dose of 1,460 μg/kg with a dosing frequency of once every 10 days. In certain aspects, the IL-7 protein is administered at a dose of 1,480 μg/kg with a dosing frequency of once every 10 days. In some aspects, the IL-7 protein is administered at a dose of 1,500 μg/kg with a dosing frequency of once every 10 days. In further aspects, the IL-7 protein is administered at a dose of 1,600 μg/kg with a dosing frequency of once every 10 days. In some aspects, the IL-7 protein is administered at a dose of 1,700 μg/kg with a dosing frequency of once every 10 days. In some aspects, the IL-7 protein is administered at a dose of 2,000 μg/kg with a dosing frequency of once every 10 days.

In some aspects, the IL-7 protein is administered at a dose of 60 μg/kg with a dosing frequency of once every 20 days. In some aspects, the IL-7 protein is administered at a dose of 120 μg/kg with a dosing frequency of once every 20 days. In some aspects, the IL-7 protein is administered at a dose of 240 μg/kg with a dosing frequency of once every 20 days. In some aspects, the IL-7 protein is administered at a dose of 480 μg/kg with a dosing frequency of once every 20 days. In some aspects, the IL-7 protein is administered at a dose of 720 μg/kg with a dosing frequency of once every 20 days. In some aspects, the IL-7 protein is administered at a dose of 960 μg/kg with a dosing frequency of once every 20 days. In some aspects, the IL-7 protein is administered at a dose of 1,200 μg/kg with a dosing frequency of once every 20 days. In some aspects, the IL-7 protein is administered at a dose of 1,300 μg/kg with a dosing frequency of once every 20 days. In some aspects, the IL-7 protein is administered at a dose of 1,400 μg/kg with a dosing frequency of once every 20 days. In other aspects, the IL-7 protein is administered at a dose of 1,420 μg/kg with a dosing frequency of once every 20 days. In certain aspects, the IL-7 protein is administered at a dose of 1,440 μg/kg with a dosing frequency of once every 20 days. In further aspects, the IL-7 protein is administered at a dose of 1,460 μg/kg with a dosing frequency of once every 20 days. In certain aspects, the IL-7 protein is administered at a dose of 1,480 μg/kg with a dosing frequency of once every 20 days. In some aspects, the IL-7 protein is administered at a dose of 1,500 μg/kg with a dosing frequency of once every 20 days. In further aspects, the IL-7 protein is administered at a dose of 1,600 μg/kg with a dosing frequency of once every 20 days. In some aspects, the IL-7 protein is administered at a dose of 1,700 μg/kg with a dosing frequency of once every 20 days. In some aspects, the IL-7 protein is administered at a dose of 2,000 μg/kg with a dosing frequency of once every 20 days.

In some aspects, the IL-7 protein is administered at a dose of 60 μg/kg with a dosing frequency of once every 30 days. In some aspects, the IL-7 protein is administered at a dose of 120 μg/kg with a dosing frequency of once every 30 days. In some aspects, the IL-7 protein is administered at a dose of 240 μg/kg with a dosing frequency of once every 30 days. In some aspects, the IL-7 protein is administered at a dose of 480 μg/kg with a dosing frequency of once every 30 days. In some aspects, the IL-7 protein is administered at a dose of 720 μg/kg with a dosing frequency of once every 30 days. In some aspects, the IL-7 protein is administered at a dose of 960 μg/kg with a dosing frequency of once every 30 days. In some aspects, the IL-7 protein is administered at a dose of 1,200 μg/kg with a dosing frequency of once every 30 days. In some aspects, the IL-7 protein is administered at a dose of 1,300 μg/kg with a dosing frequency of once every 30 days. In some aspects, the IL-7 protein is administered at a dose of 1,400 μg/kg with a dosing frequency of once every 30 days. In other aspects, the IL-7 protein is administered at a dose of 1,420 μg/kg with a dosing frequency of once every 30 days. In certain aspects, the IL-7 protein is administered at a dose of 1,440 μg/kg with a dosing frequency of once every 30 days. In further aspects, the IL-7 protein is administered at a dose of 1,460 μg/kg with a dosing frequency of once every 30 days. In certain aspects, the IL-7 protein is administered at a dose of 1,480 μg/kg with a dosing frequency of once every 30 days. In some aspects, the IL-7 protein is administered at a dose of 1,500 μg/kg with a dosing frequency of once every 30 days. In further aspects, the IL-7 protein is administered at a dose of 1,600 μg/kg with a dosing frequency of once every 30 days. In some aspects, the IL-7 protein is administered at a dose of 1,700 μg/kg with a dosing frequency of once every 30 days. In some aspects, the IL-7 protein is administered at a dose of 2,000 μg/kg with a dosing frequency of once every 30 days.

In some aspects, the IL-7 protein is administered at a dose of 60 μg/kg with a dosing frequency of once every 40 days. In some aspects, the IL-7 protein is administered at a dose of 120 μg/kg with a dosing frequency of once every 40 days. In some aspects, the IL-7 protein is administered at a dose of 240 μg/kg with a dosing frequency of once every 40 days. In some aspects, the IL-7 protein is administered at a dose of 480 μg/kg with a dosing frequency of once every 40 days. In some aspects, the IL-7 protein is administered at a dose of 720 μg/kg with a dosing frequency of once every 40 days. In some aspects, the IL-7 protein is administered at a dose of 960 μg/kg with a dosing frequency of once every 40 days. In some aspects, the IL-7 protein is administered at a dose of 1,200 μg/kg with a dosing frequency of once every 40 days. In some aspects, the IL-7 protein is administered at a dose of 1,300 μg/kg with a dosing frequency of once every 40 days. In some aspects, the IL-7 protein is administered at a dose of 1,400 μg/kg with a dosing frequency of once every 40 days. In other aspects, the IL-7 protein is administered at a dose of 1,420 μg/kg with a dosing frequency of once every 40 days. In certain aspects, the IL-7 protein is administered at a dose of 1,440 μg/kg with a dosing frequency of once every 40 days. In further aspects, the IL-7 protein is administered at a dose of 1,460 μg/kg with a dosing frequency of once every 40 days. In certain aspects, the IL-7 protein is administered at a dose of 1,480 μg/kg with a dosing frequency of once every 40 days. In some aspects, the IL-7 protein is administered at a dose of 1,500 μg/kg with a dosing frequency of once every 40 days. In further aspects, the IL-7 protein is administered at a dose of 1,600 μg/kg with a dosing frequency of once every 40 days. In some aspects, the IL-7 protein is administered at a dose of 1,700 μg/kg with a dosing frequency of once every 40 days. In some aspects, the IL-7 protein is administered at a dose of 2,000 μg/kg with a dosing frequency of once every 40 days.

In some aspects, the IL-7 protein is administered at a dose of 60 μg/kg with a dosing frequency of once every 50 days. In some aspects, the IL-7 protein is administered at a dose of 120 μg/kg with a dosing frequency of once every 50 days. In some aspects, the IL-7 protein is administered at a dose of 240 μg/kg with a dosing frequency of once every 50 days. In some aspects, the IL-7 protein is administered at a dose of 480 μg/kg with a dosing frequency of once every 50 days. In some aspects, the IL-7 protein is administered at a dose of 720 μg/kg with a dosing frequency of once every 50 days. In some aspects, the IL-7 protein is administered at a dose of 960 μg/kg with a dosing frequency of once every 50 days. In some aspects, the IL-7 protein is administered at a dose of 1,200 μg/kg with a dosing frequency of once every 50 days. In some aspects, the IL-7 protein is administered at a dose of 1,300 μg/kg with a dosing frequency of once every 50 days. In some aspects, the IL-7 protein is administered at a dose of 1,400 μg/kg with a dosing frequency of once every 50 days. In other aspects, the IL-7 protein is administered at a dose of 1,420 μg/kg with a dosing frequency of once every 50 days. In certain aspects, the IL-7 protein is administered at a dose of 1,440 μg/kg with a dosing frequency of once every 50 days. In further aspects, the IL-7 protein is administered at a dose of 1,460 μg/kg with a dosing frequency of once every 50 days. In certain aspects, the IL-7 protein is administered at a dose of 1,480 μg/kg with a dosing frequency of once every 50 days. In some aspects, the IL-7 protein is administered at a dose of 1,500 μg/kg with a dosing frequency of once every 50 days. In further aspects, the IL-7 protein is administered at a dose of 1,600 μg/kg with a dosing frequency of once every 50 days. In some aspects, the IL-7 protein is administered at a dose of 1,700 μg/kg with a dosing frequency of once every 50 days. In some aspects, the IL-7 protein is administered at a dose of 2,000 μg/kg with a dosing frequency of once every 50 days.

In some aspects, the IL-7 protein is administered at a dose of 60 μg/kg with a dosing frequency of once every 60 days. In some aspects, the IL-7 protein is administered at a dose of 120 μg/kg with a dosing frequency of once every 60 days. In some aspects, the IL-7 protein is administered at a dose of 240 μg/kg with a dosing frequency of once every 60 days. In some aspects, the IL-7 protein is administered at a dose of 480 μg/kg with a dosing frequency of once every 60 days. In some aspects, the IL-7 protein is administered at a dose of 720 μg/kg with a dosing frequency of once every 60 days. In some aspects, the IL-7 protein is administered at a dose of 960 μg/kg with a dosing frequency of once every 60 days. In some aspects, the IL-7 protein is administered at a dose of 1,200 μg/kg with a dosing frequency of once every 60 days. In some aspects, the IL-7 protein is administered at a dose of 1,300 μg/kg with a dosing frequency of once every 60 days. In some aspects, the IL-7 protein is administered at a dose of 1,400 μg/kg with a dosing frequency of once every 60 days. In other aspects, the IL-7 protein is administered at a dose of 1,420 μg/kg with a dosing frequency of once every 60 days. In certain aspects, the IL-7 protein is administered at a dose of 1,440 μg/kg with a dosing frequency of once every 60 days. In further aspects, the IL-7 protein is administered at a dose of 1,460 μg/kg with a dosing frequency of once every 60 days. In certain aspects, the IL-7 protein is administered at a dose of 1,480 μg/kg with a dosing frequency of once every 60 days. In some aspects, the IL-7 protein is administered at a dose of 1,500 μg/kg with a dosing frequency of once every 60 days. In further aspects, the IL-7 protein is administered at a dose of 1,600 μg/kg with a dosing frequency of once every 60 days. In some aspects, the IL-7 protein is administered at a dose of 1,700 μg/kg with a dosing frequency of once every 60 days. In some aspects, the IL-7 protein is administered at a dose of 2,000 μg/kg with a dosing frequency of once every 60 days.

In some aspects, the IL-7 protein is administered at a dose of 60 μg/kg with a dosing frequency of once every 70 days. In some aspects, the IL-7 protein is administered at a dose of 120 μg/kg with a dosing frequency of once every 70 days. In some aspects, the IL-7 protein is administered at a dose of 240 μg/kg with a dosing frequency of once every 70 days. In some aspects, the IL-7 protein is administered at a dose of 480 μg/kg with a dosing frequency of once every 70 days. In some aspects, the IL-7 protein is administered at a dose of 720 μg/kg with a dosing frequency of once every 70 days. In some aspects, the IL-7 protein is administered at a dose of 960 μg/kg with a dosing frequency of once every 70 days. In some aspects, the IL-7 protein is administered at a dose of 1,200 μg/kg with a dosing frequency of once every 70 days. In some aspects, the IL-7 protein is administered at a dose of 1,300 μg/kg with a dosing frequency of once every 70 days. In some aspects, the IL-7 protein is administered at a dose of 1,400 μg/kg with a dosing frequency of once every 70 days. In other aspects, the IL-7 protein is administered at a dose of 1,420 μg/kg with a dosing frequency of once every 70 days. In certain aspects, the IL-7 protein is administered at a dose of 1,440 μg/kg with a dosing frequency of once every 70 days. In further aspects, the IL-7 protein is administered at a dose of 1,460 μg/kg with a dosing frequency of once every 70 days. In certain aspects, the IL-7 protein is administered at a dose of 1,480 μg/kg with a dosing frequency of once every 70 days. In some aspects, the IL-7 protein is administered at a dose of 1,500 μg/kg with a dosing frequency of once every 70 days. In further aspects, the IL-7 protein is administered at a dose of 1,600 μg/kg with a dosing frequency of once every 70 days. In some aspects, the IL-7 protein is administered at a dose of 1,700 μg/kg with a dosing frequency of once every 70 days. In some aspects, the IL-7 protein is administered at a dose of 2,000 μg/kg with a dosing frequency of once every 70 days.

In some aspects, the IL-7 protein is administered at a dose of 60 μg/kg with a dosing frequency of once every 80 days. In some aspects, the IL-7 protein is administered at a dose of 120 μg/kg with a dosing frequency of once every 80 days. In some aspects, the IL-7 protein is administered at a dose of 240 μg/kg with a dosing frequency of once every 80 days. In some aspects, the IL-7 protein is administered at a dose of 480 μg/kg with a dosing frequency of once every 80 days. In some aspects, the IL-7 protein is administered at a dose of 720 μg/kg with a dosing frequency of once every 80 days. In some aspects, the IL-7 protein is administered at a dose of 960 μg/kg with a dosing frequency of once every 80 days. In some aspects, the IL-7 protein is administered at a dose of 1,200 μg/kg with a dosing frequency of once every 80 days. In some aspects, the IL-7 protein is administered at a dose of 1,300 μg/kg with a dosing frequency of once every 80 days. In some aspects, the IL-7 protein is administered at a dose of 1,400 μg/kg with a dosing frequency of once every 80 days. In other aspects, the IL-7 protein is administered at a dose of 1,420 μg/kg with a dosing frequency of once every 80 days. In certain aspects, the IL-7 protein is administered at a dose of 1,440 μg/kg with a dosing frequency of once every 80 days. In further aspects, the IL-7 protein is administered at a dose of 1,460 μg/kg with a dosing frequency of once every 80 days. In certain aspects, the IL-7 protein is administered at a dose of 1,480 μg/kg with a dosing frequency of once every 80 days. In some aspects, the IL-7 protein is administered at a dose of 1,500 μg/kg with a dosing frequency of once every 80 days. In further aspects, the IL-7 protein is administered at a dose of 1,600 μg/kg with a dosing frequency of once every 80 days. In some aspects, the IL-7 protein is administered at a dose of 1,700 μg/kg with a dosing frequency of once every 80 days. In some aspects, the IL-7 protein is administered at a dose of 2,000 μg/kg with a dosing frequency of once every 80 days.

In some aspects, the IL-7 protein is administered at a dose of 60 μg/kg with a dosing frequency of once every 90 days. In some aspects, the IL-7 protein is administered at a dose of 120 μg/kg with a dosing frequency of once every 90 days. In some aspects, the IL-7 protein is administered at a dose of 240 μg/kg with a dosing frequency of once every 90 days. In some aspects, the IL-7 protein is administered at a dose of 480 μg/kg with a dosing frequency of once every 90 days. In some aspects, the IL-7 protein is administered at a dose of 720 μg/kg with a dosing frequency of once every 90 days. In some aspects, the IL-7 protein is administered at a dose of 960 μg/kg with a dosing frequency of once every 90 days. In some aspects, the IL-7 protein is administered at a dose of 1,200 μg/kg with a dosing frequency of once every 90 days. In some aspects, the IL-7 protein is administered at a dose of 1,300 μg/kg with a dosing frequency of once every 90 days. In some aspects, the IL-7 protein is administered at a dose of 1,400 μg/kg with a dosing frequency of once every 90 days. In other aspects, the IL-7 protein is administered at a dose of 1,420 μg/kg with a dosing frequency of once every 90 days. In certain aspects, the IL-7 protein is administered at a dose of 1,440 μg/kg with a dosing frequency of once every 90 days. In further aspects, the IL-7 protein is administered at a dose of 1,460 μg/kg with a dosing frequency of once every 90 days. In certain aspects, the IL-7 protein is administered at a dose of 1,480 μg/kg with a dosing frequency of once every 90 days. In some aspects, the IL-7 protein is administered at a dose of 1,500 μg/kg with a dosing frequency of once every 90 days. In further aspects, the IL-7 protein is administered at a dose of 1,600 μg/kg with a dosing frequency of once every 90 days. In some aspects, the IL-7 protein is administered at a dose of 1,700 μg/kg with a dosing frequency of once every 90 days. In some aspects, the IL-7 protein is administered at a dose of 2,000 μg/kg with a dosing frequency of once every 90 days.

In some aspects, the IL-7 protein is administered at a dose of 60 μg/kg with a dosing frequency of once every 100 days. In some aspects, the IL-7 protein is administered at a dose of 120 μg/kg with a dosing frequency of once every 100 days. In some aspects, the IL-7 protein is administered at a dose of 240 μg/kg with a dosing frequency of once every 100 days. In some aspects, the IL-7 protein is administered at a dose of 480 μg/kg with a dosing frequency of once every 100 days. In some aspects, the IL-7 protein is administered at a dose of 720 μg/kg with a dosing frequency of once every 100 days. In some aspects, the IL-7 protein is administered at a dose of 960 μg/kg with a dosing frequency of once every 100 days. In some aspects, the IL-7 protein is administered at a dose of 1,200 μg/kg with a dosing frequency of once every 100 days. In some aspects, the IL-7 protein is administered at a dose of 1,300 μg/kg with a dosing frequency of once every 100 days. In some aspects, the IL-7 protein is administered at a dose of 1,400 μg/kg with a dosing frequency of once every 100 days. In other aspects, the IL-7 protein is administered at a dose of 1,420 μg/kg with a dosing frequency of once every 100 days. In certain aspects, the IL-7 protein is administered at a dose of 1,440 μg/kg with a dosing frequency of once every 100 days. In further aspects, the IL-7 protein is administered at a dose of 1,460 μg/kg with a dosing frequency of once every 100 days. In certain aspects, the IL-7 protein is administered at a dose of 1,480 μg/kg with a dosing frequency of once every 100 days. In some aspects, the IL-7 protein is administered at a dose of 1,500 μg/kg with a dosing frequency of once every 100 days. In further aspects, the IL-7 protein is administered at a dose of 1,600 μg/kg with a dosing frequency of once every 100 days. In some aspects, the IL-7 protein is administered at a dose of 1,700 μg/kg with a dosing frequency of once every 100 days. In some aspects, the IL-7 protein is administered at a dose of 2,000 μg/kg with a dosing frequency of once every 100 days.

### III. IL-7 Proteins Useful for the Disclosure

Disclosed herein are IL-7 proteins that can be used (alone or in combination with an additional agent disclosed herein) with the various methods disclosed herein (e.g., method of mobilizing HSCs, method of increasing the recovery of HSCs from peripheral blood, method of reconstituting a hematopoietic compartment, method of treating an abnormality in a hematopoietic process and/or cancer). In some aspects, IL-7 protein useful for the present uses can be wild-type IL-7 or modified IL-7 (i.e., not wild-type IL-7 protein) (e.g., IL-7 variant, IL-7 functional fragment, IL-7 derivative, or any combination thereof, e.g., fusion protein, chimeric protein, etc.) as long as the IL-7 protein contains one or more biological activities of IL-7, e.g., capable of binding to IL-7R, e.g., inducing early T-cell development, promoting T-cell homeostasis. See ElKassar and Gress. J Immunotoxicol. 2010 March; 7(1): 1-7. In some aspects, an IL-7 protein of the present disclosure is not a wild-type IL-7 protein (i.e., comprises one or more modifications). Non-limiting examples of such modifications can include an oligopeptide and/or a half-life extending moiety. See WO 2016/200219, which is herein incorporated by reference in its entirety.

IL-7 binds to its receptor which is composed of the two chains IL-7Rα (CD127), shared with the thymic stromal lymphopoietin (TSLP) (Ziegler and Liu, 2006), and the common γ chain (CD132) for IL-2, IL-15, IL-9 and IL-21. Whereas γc is expressed by most hematopoietic cells, IL-7Rα is nearly exclusively expressed on lymphoid cells. After binding to its receptor, IL-7 signals through two different pathways: Jak-Stat (Janus kinase-Signal transducer and activator of transcription) and PI3K/Akt responsible for differentiation and survival, respectively. The absence of IL-7 signaling is responsible for a reduced thymic cellularity as observed in mice that have received an anti-IL-7 neutralizing monoclonal antibody (MAb); Grabstein et al., 1993), in IL-7−/− (von Freeden-Jeffry et al., 1995), IL-7Rα−/− (Peschon et al., 1994; Maki et al., 1996), γc−/− (Malissen et al., 1997), and Jak3−/− mice (Park et al., 1995). In the absence of IL-7 signaling, mice lack T-, B-, and NK-T cells. IL-7α−/− mice (Peschon et al., 1994) have a similar but more severe phenotype than IL-7−/− mice (von Freeden-Jeffry et al., 1995), possibly because TSLP signaling is also abrogated in IL-7α−/− mice. IL-7 is required for the development of γδ cells (Maki et al., 1996) and NK-T cells (Boesteanu et al., 1997).

In some aspects, an IL-7 protein useful for the present disclosure comprises an amino acid sequence as set forth in any one of SEQ ID NOs: 1 to 6. In other aspects, the IL-7 protein comprises an amino acid sequence having a sequence identity of about 70%, about 75%, about 80%, about 85%, about 90%, about 91%, about 92%, about 93%, about 94%, about 95%, about 96%, about 97%, about 98%, or about 99% or higher, to a sequence of SEQ ID NOS: 1 to 6.

In some aspects, the IL-7 protein includes a modified IL-7 or a fragment thereof, wherein the modified IL-7 or the fragment retains one or more biological activities of wild-type IL-7. In some aspects, the IL-7 protein can be derived from humans, rats, mice, monkeys, cows, or sheep.

In some aspects, the human IL-7 can have an amino acid sequence represented by SEQ ID NO: 1 (Genbank Accession No. P13232); the rat IL-7 can have an amino acid sequence represented by SEQ ID NO: 2 (Genbank Accession No. P56478); the mouse IL-7 can have an amino acid sequence represented by SEQ ID NO: 3 (Genbank Accession No. P10168); the monkey IL-7 may have an amino acid sequence represented by SEQ ID NO: 4 (Genbank Accession No. NP 001279008); the cow IL-7 can have an amino acid sequence represented by SEQ ID NO: 5 (Genbank Accession No. P26895), and the sheep IL-7 can have an amino acid sequence represented by SEQ ID NO: 6 (Genbank Accession No. Q28540).

In some aspects, an IL-7 protein useful for the present disclosure comprises an IL-7 fusion protein. In certain aspects, an IL-7 fusion protein comprises (i) an oligopeptide and (i) an IL-7 or a variant thereof. In some aspects, the oligopeptide is linked to the N-terminal region of the IL-7 or a variant thereof.

In some aspects, an oligopeptide disclosed herein consists of 1 to 10 amino acids. In certain aspects, an oligopeptide consists of at least 1, at least 2, at least 3, at least 4, at least 5, at least 6, at least 7, at least 8, at least 9, or 10 amino acids. In some aspects, one or more amino acids of an oligopeptide are selected from the group consisting of methionine, glycine, and combinations thereof. In certain aspects, an oligopeptide is selected from the group consisting of methionine (M), glycine (G), methionine-methionine (MM), glycine-glycine (GG), methionine-glycine (MG), glycine-methionine (GM), methionine-methionine-methionine (MMM), methionine-methionine-glycine (MMG), methionine-glycine-methionine (MGM), glycine-methionine-methionine (GMM), methionine-glycine-glycine (MGG), glycine-methionine-glycine (GMG), glycine-glycine-methionine (GGM), glycine-glycine-glycine (GGG), methionine-glycine-glycine-methionine (MGGM) (SEQ ID NO: 41), methionine-methionine-glycine-glycine (MMGG) (SEQ ID NO: 42), glycine-glycine-methionine-methionine (GGMM) (SEQ ID NO: 43), methionine-glycine-methionine-glycine (MGMG) (SEQ ID NO: 44), glycine-methionine-methionine-glycine (GMMG) (SEQ ID NO: 45), glycine-glycine-glycine-methionine (GGGM) (SEQ ID NO: 46), methionine-glycine-glycine-glycine (MGGG) (SEQ ID NO: 47), glycine-methionine-glycine-glycine (GMGG) (SEQ ID NO: 48), glycine-glycine-methionine-glycine (GGMG) (SEQ ID NO: 49), glycine-glycine-methionine-methionine-methionine (GGMMM) (SEQ ID NO: 50), glycine-glycine-glycine-methionine-methionine (GGGMM) (SEQ ID NO: 51), glycine-glycine-glycine-glycine-methionine (GGGGM) (SEQ ID NO: 52), methionine-glycine-methionine-methionine-methionine (MGMMM) (SEQ ID NO: 53), methionine-glycine-glycine-methionine-methionine (MGGMM) (SEQ ID NO: 54), methionine-glycine-glycine-glycine-methionine (MGGGM) (SEQ ID NO: 55), methionine-methionine-glycine-methionine-methionine (MMGMM) (SEQ ID NO: 56), methionine-methionine-glycine-glycine-methionine (MMGGM) (SEQ ID NO: 57), methionine-methionine-glycine-glycine-glycine (MMGGG) (SEQ ID NO: 58), methionine-methionine-methionine-glycine-methionine (MMMGM) (SEQ ID NO: 59), methionine-glycine-methionine-glycine-methionine (MGMGM) (SEQ ID NO: 60), glycine-methionine-glycine-methionine-glycine (GMGMG) (SEQ ID NO: 61), glycine-methionine-methionine-methionine-glycine (GMMMG) (SEQ ID NO: 62), glycine-glycine-methionine-glycine-methionine (GGMGM) (SEQ ID NO: 63), glycine-glycine-methionine-methionine-glycine (GGMMG) (SEQ ID NO: 64), glycine-methionine-methionine-glycine-methionine (GMMGM) (SEQ ID NO: 65), methionine-glycine-methionine-methionine-glycine (MGMMG) (SEQ ID NO: 66), glycine-methionine-glycine-glycine-methionine (GMGGM) (SEQ ID NO: 67), methionine-methionine-glycine-methionine-glycine (MMGMG) (SEQ ID NO: 68), glycine-methionine-methionine-glycine-glycine (GMMGG) (SEQ ID NO: 69), glycine-methionine-glycine-glycine-glycine (GMGGG) (SEQ ID NO: 70), glycine-glycine-methionine-glycine-glycine (GGMGG) (SEQ ID NO: 71), glycine-glycine-glycine-glycine-glycine (GGGGG) (SEQ ID NO: 72), or combinations thereof. In some aspects, an oligopeptide is methionine-glycine-methionine (MGM).

In some aspects, an IL-7 fusion protein comprises (i) an IL-7 or a variant thereof, and (ii) a half-life extending moiety. In some aspects, a half-life extending moiety extends the half-life of the IL-7 or variant thereof. In some aspects, a half-life extending moiety is linked to the C-terminal region of an IL-7 or a variant thereof.

In some aspects, an IL-7 fusion protein comprises (i) IL-7 (a first domain), (ii) a second domain that includes an amino acid sequence having 1 to 10 amino acid residues consisting of methionine, glycine, or a combination thereof, e.g., MGM, and (iii) a third domain comprising a half-life extending moiety. In some aspects, the half-life extending moiety can be linked to the N-terminal or the C-terminal of the first domain or the second domain. Additionally, the IL-7 including the first domain and the second domain can be linked to both terminals of the third domain.

Non-limiting examples of half-life extending moieties include: Fc, albumin, an albumin-binding polypeptide, Pro/Ala/Ser (PAS), a C-terminal peptide (CTP) of the 0 subunit of human chorionic gonadotropin, polyethylene glycol (PEG), long unstructured hydrophilic sequences of amino acids (XTEN), hydroxyethyl starch (HES), an albumin-binding small molecule, and combinations thereof.

In some aspects, a half-life extending moiety is Fc. In certain aspects, Fc is from a modified immunoglobulin in which the antibody-dependent cellular cytotoxicity (ADCC) or complement-dependent cytotoxicity (CDC) weakened due to the modification in the binding affinity with the Fc receptor and/or a complement. In some aspects, the modified immunoglobulin can be selected from the group consisting of IgG1, IgG2, IgG3, IgG4, IgA1, IgA2, IgD, IgE, and a combination thereof. In some aspects, an Fc is a hybrid Fc (“hFc” or “hyFc”), comprising a hinge region, a CH2 domain, and a CH3 domain. In certain aspects, a hinge region of a hybrid Fc disclosed herein comprises a human IgD hinge region. In certain aspects, a CH2 domain of a hybrid Fc comprises a part of human IgD CH2 domain and a part of human IgG4 CH2 domain. In certain aspects, a CH3 domain of a hybrid Fc comprises a part of human IgG4 CH3 domain. Accordingly, in some aspects, a hybrid Fc disclosed herein comprises a hinge region, a CH2 domain, and a CH3 domain, wherein the hinge region comprises a human IgD hinge region, wherein the CH2 domain comprises a part of human IgD CH2 domain and a part of human IgG4 CH2 domain, and wherein the CH3 domain comprises a part of human IgG4 CH3 domain.

In some aspects, an Fc disclosed herein can be an Fc variant. As used herein, the term “Fc variant” refers to an Fc which was prepared by substituting a part of the amino acids among the Fc region or by combining the Fc regions of different kinds. The Fc region variant can prevent from being cut off at the hinge region. Specifically, in some aspects, a Fc variant comprises modifications at the 144^{th} amino acid and/or 145^{th} amino acid of SEQ ID NO: 9. In certain aspects, the 144^{th} amino acid (K) and/or the 145^{th} amino acid (K) is substituted with G or S.

In some aspects, an Fc or an Fc variant disclosed herein can be represented by the following formula: N′—(Z1)p-Y—Z2-Z3-Z4-C, wherein:
N′ comprises the N-terminus;
Z1 comprises an amino acid sequence having 5 to 9 consecutive amino acid residues from the amino acid residue at position 98 toward the N-terminal, among the amino acid residues at positions from 90 to 98 of SEQ ID NO: 7;
Y comprises an amino acid sequence having 5 to 64 consecutive amino acid residues from the amino acid residue at position 162 toward the N-terminal, among the amino acid residues at positions from 99 to 162 of SEQ ID NO: 7;
Z2 comprises an amino acid sequence having 4 to 37 consecutive amino acid residues from the amino acid residue at position 163 toward the C-terminal, among the amino acid residues at positions from 163 to 199 of SEQ ID NO: 7;
Z3 comprises an amino acid sequence having 71 to 106 consecutive amino acid residues from the amino acid residue at position 220 toward the N-terminal, among the amino acid residues at positions from 115 to 220 of SEQ ID NO: 8; and
Z4 comprises an amino acid sequence having 80 to 107 consecutive amino acid residues from the amino acid residue at position 221 toward the C-terminal, among the amino acid residues at positions from 221 to 327 of SEQ ID NO: 8.

In some aspects, a Fc region disclosed herein can include the amino acid sequence of SEQ ID NO: 9 (hyFc), SEQ ID NO: 10 (hyFcM1), SEQ ID NO: 11 (hyFcM2), SEQ ID NO: 12 (hyFcM3), or SEQ ID NO: 13 (hyFcM4). In some aspects, the Fc region can include the amino acid sequence of SEQ ID NO: 14 (a non-lytic mouse Fc).

Other non-limiting examples of Fc regions that can be used with the present disclosure are described in U.S. Pat. No. 7,867,491, which is herein incorporated by reference in its entirety.

In some aspects, an IL-7 fusion protein disclosed herein comprises both an oligopeptide and a half-life extending moiety.

In some aspects, an IL-7 protein can be fused to albumin, a variant, or a fragment thereof. Examples of the IL-7-albumin fusion protein can be found at International Application Publication No. WO 2011/124718 A1. In some aspects, an IL-7 protein is fused to a pre-pro-B cell Growth Stimulating Factor (PPBSF), optionally by a flexible linker. See US 2002/0058791A1. In other aspects, an IL-7 protein useful for the disclosure is an IL-7 conformer that has a particular three dimensional structure. See US 2005/0249701 A1. In some aspects, an IL-7 protein can be fused to an Ig chain, wherein amino acid residues 70 and 91 in the IL-7 protein are glycosylated the amino acid residue 116 in the IL-7 protein is non-glycosylated. See U.S. Pat. No. 7,323,549 B2. In some aspects, an IL-7 protein that does not contain potential T-cell epitopes (thereby to reduce anti-IL-7 T-cell responses) can also be used for the present disclosure. See US 2006/0141581 A1. In other aspects, an IL-7 protein that has one or more amino acid residue mutations in carboxy-terminal helix D region can be used for the present disclosure. The IL-7 mutant can act as IL-7R partial agonist despite lower binding affinity for the receptor. See US 2005/0054054A1. Any IL-7 proteins described in the above listed patents or publications are incorporated herein by reference in their entireties.

In addition, non-limiting examples of additional IL-7 proteins useful for the present disclosure are described in U.S. Pat. Nos. 7,708,985, 8,034,327, 8,153,114, 7,589,179, 7,323,549, 7,960,514, 8,338,575, 7,118,754, 7,488,482, 7,670,607, 6,730,512, WO0017362, GB2434578A, WO 2010/020766 A2, WO91/01143, Beq et al., Blood, vol. 114 (4), 816, 23 Jul. 2009, Kang et al., J. Virol. Doi:10.1128/JVI.02768-15, Martin et al., Blood, vol. 121 (22), 4484, May 30, 2013, McBride et al., Acta Oncologica, 34:3, 447-451, Jul. 8, 2009, and Xu et al., Cancer Science, 109: 279-288, 2018, which are incorporated herein by reference in their entireties.

The present disclosure is directed to a method for treating a tumor (or a cancer) in a subject in need thereof, comprising administering to the subject an effective amount of an interleukin-7 (IL-7) protein in combination with an effective amount of an immune checkpoint inhibitor. Non-limiting examples of immune checkpoint inhibitors that can be used with the current methods include an anti-PD-1 antibody, anti-PD-L1 antibody, anti-CTLA-4 antibody, and combinations thereof.

In some aspects, an oligopeptide disclosed herein is directly linked to the N-terminal region of IL-7 or a variant thereof. In other aspects, an oligopeptide is linked to the N-terminal region via a linker. In some aspects, a half-life extending moiety disclosed herein is directly linked to the C-terminal region of IL-7 or a variant thereof. In certain aspects, a half-life extending moiety is linked to the C-terminal region via a linker. In some aspects, a linker is a peptide linker. In certain aspects, a peptide linker comprises a peptide of 10 to 20 amino acid residues consisting of Gly and Ser residues. In some aspects, a linker is an albumin linker. In some aspects, a linker is a chemical bond. In certain aspects, a chemical bond comprises a disulfide bond, a diamine bond, a sulfide-amine bond, a carboxy-amine bond, an ester bond, a covalent bond, or combinations thereof. When the linker is a peptide linker, in some aspects, the connection can occur in any linking region. They may be coupled using a crosslinking agent known in the art. In some aspects, examples of the crosslinking agent can include N-hydroxy succinimide esters such as 1,1-bis(diazoacetyl)-2-phenylethane, glutaraldehyde, and 4-azidosalicylic acid; imido esters including disuccinimidyl esters such as 3,3′-dithiobis (succinimidyl propionate), and bifunctional maleimides such as bis-Nmaleimido-1,8-octane, but is not limited thereto.

In some aspects, an IL-7 (or variant thereof) portion of IL-7 fusion protein disclosed herein comprises an amino sequence that is at least 70%, at least 75%, at least 80%, at least 85%, at least 90%, at least 91%, at least 92%, at least 93%, at least 94%, at least 95%, at least 96%, at least 97%, at least 98%, at least 98%, or at least 99% identical to an amino acid sequence set forth in SEQ ID NOs: 15-20. In certain aspects, an IL-7 (or variant thereof) portion of IL-7 fusion protein disclosed herein comprises the amino acid sequence set forth in SEQ ID NOs: 15-20.

In some aspects, an IL-7 fusion protein comprises: a first domain including a polypeptide having the activity of IL-7 or a similar activity thereof; a second domain comprising an amino acid sequence having 1 to 10 amino acid residues consisting of methionine, glycine, or a combination thereof; and a third domain, which is an Fc region of modified immunoglobulin, coupled to the C-terminal of the first domain.

In some aspects, an IL-7 fusion protein that can be used with the present methods comprises an amino sequence that is at least 70%, at least 75%, at least 80%, at least 85%, at least 90%, at least 91%, at least 92%, at least 93%, at least 94%, at least 95%, at least 96%, at least 97%, at least 98%, at least 98%, or at least 99% identical to an amino acid sequence set forth in SEQ ID NOs: 21-25. In certain aspects, an IL-7 fusion protein of the present disclosure comprises the amino acid sequence set forth in SEQ ID NOs: 21-25. In further aspects, an IL-7 fusion protein disclosed herein comprises the amino acid sequence set forth in SEQ ID NOs: 26 and 27.

### IV. Nucleic Acids, Vectors, Host Cells

Further aspect described herein pertains to one or more nucleic acid molecules that encode a therapeutic agent described herein (e.g., an IL-7 protein). The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al., ed. (1987) Current Protocols in Molecular Biology, Greene Publishing and Wiley Interscience, New York. A nucleic acid described herein can be, for example, DNA or RNA and can or cannot contain intronic sequences. In a certain aspects, the nucleic acid is a cDNA molecule. Nucleic acids described herein can be obtained using standard molecular biology techniques known in the art.

Certain nucleic acid molecules disclosed herein are those encoding an IL-7 protein (e.g., disclosed herein). Exemplary nucleic acid sequences encoding an IL-7 protein disclosed herein are set forth in SEQ ID NOs: 29-39.

In some aspects, the present disclosure provides a vector comprising an isolated nucleic acid molecule encoding a therapeutic agent disclosed herein (e.g., an IL-7 protein). In some aspects, a vector can be used for gene therapy.

When used as a gene therapy (e.g., in humans), a nucleic acid encoding a therapeutic agent disclosed herein (e.g., an IL-7 protein) can be administered at a dosage in the range of 0.1 mg to 200 mg. In certain aspects, the dosage is in the range of 0.6 mg to 100 mg. In further aspects, the dosage is in the range of 1.2 mg to 50 mg.

Suitable vectors for the disclosure include expression vectors, viral vectors, and plasmid vectors. In some aspects, the vector is a viral vector.

As used herein, an expression vector refers to any nucleic acid construct which contains the necessary elements for the transcription and translation of an inserted coding sequence, or in the case of an RNA viral vector, the necessary elements for replication and translation, when introduced into an appropriate host cell. Expression vectors can include plasmids, phagemids, viruses, and derivatives thereof.

As used herein, viral vectors include, but are not limited to, nucleic acid sequences from the following viruses: retrovirus, such as Moloney murine leukemia virus, Harvey murine sarcoma virus, murine mammary tumor virus, and Rous sarcoma virus; lentivirus; adenovirus; adeno-associated virus; SV40-type viruses; polyomaviruses; Epstein-Barr viruses; papilloma viruses; herpes virus; vaccinia virus; polio virus; and RNA virus such as a retrovirus. One can readily employ other vectors well-known in the art. Certain viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA.

In some aspects, a vector is derived from an adeno-associated virus. In other aspects, a vector is derived from a lentivirus. Examples of the lentiviral vectors are disclosed in WO9931251, W09712622, W09817815, W09817816, and WO9818934, each which is incorporated herein by reference in its entirety.

Other vectors include plasmid vectors. Plasmid vectors have been extensively described in the art and are well-known to those of skill in the art. See, e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, CA.). Other plasmids are well-known to those of ordinary skill in the art. Additionally, plasmids can be custom designed using standard molecular biology techniques to remove and/or add specific fragments of DNA.

Also encompassed by the present disclosure is a method for making a therapeutic agent disclosed herein (e.g., an IL-7 protein). In some aspects, such a method can comprise expressing the therapeutic agent (e.g., an IL-7 protein) in a cell comprising a nucleic acid molecule encoding the therapeutic agent, e.g., SEQ ID NOs: 29-39. Additional details regarding the method for making an IL-7 protein disclosed herein are provided, e.g., in WO 2016/200219, which is herein incorporated by reference in its entirety. Host cells comprising these nucleotide sequences are encompassed herein. Non-limiting examples of host cell that can be used include immortal hybridoma cell, NSIO myeloma cell, 293 cell, Chinese hamster ovary (CHO) cell, HeLa cell, human amniotic fluid-derived cell (CapT cell), COS cell, or combinations thereof.

### V. Pharmaceutical Compositions

Further provided herein are compositions comprising one or more therapeutic agents (e.g., an IL-7 protein alone or in combination with an additional agent disclosed herein, e.g., G-CSF and/or AMD3100) having the desired degree of purity in a physiologically acceptable carrier, excipient or stabilizer (Remington's Pharmaceutical Sciences (1990) Mack Publishing Co., Easton, PA). In some aspects, a composition disclosed herein comprises an IL-7 protein or an additional agent (e.g., G-CSF and/or AMD3100). As disclosed herein, such compositions can be used in combination (e.g., a first composition comprising an IL-7 protein and a second composition comprising an additional agent, such as G-CSF and/or AMD3100). In other aspects, a composition disclosed herein can comprise both an IL-7 protein and an additional agent (e.g., G-CSF and/or AMD3100).

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

In some aspects, a composition disclosed herein (e.g., comprising an IL-7 protein or an immune checkpoint inhibitor) comprises one or more additional components selected from: a bulking agent, stabilizing agent, surfactant, buffering agent, or combinations thereof.

Buffering agents useful for the current disclosure can be a weak acid or base used to maintain the acidity (pH) of a solution near a chosen value after the addition of another acid or base. Suitable buffering agents can maximize the stability of the pharmaceutical compositions by maintaining pH control of the composition. Suitable buffering agents can also ensure physiological compatibility or optimize solubility. Rheology, viscosity and other properties can also be dependent on the pH of the composition. Common buffering agents include, but are not limited to, a Tris buffer, a Tris-Cl buffer, a histidine buffer, a TAE buffer, a HEPES buffer, a TBE buffer, a sodium phosphate buffer, a MES buffer, an ammonium sulfate buffer, a potassium phosphate buffer, a potassium thiocyanate buffer, a succinate buffer, a tartrate buffer, a DIPSO buffer, a HEPPSO buffer, a POPSO buffer, a PIPES buffer, a PBS buffer, a MOPS buffer, an acetate buffer, a phosphate buffer, a cacodylate buffer, a glycine buffer, a sulfate buffer, an imidazole buffer, a guanidine hydrochloride buffer, a phosphate-citrate buffer, a borate buffer, a malonate buffer, a 3-picoline buffer, a 2-picoline buffer, a 4-picoline buffer, a 3,5-lutidine buffer, a 3,4-lutidine buffer, a 2,4-lutidine buffer, a Aces, a diethylmalonate buffer, a N-methylimidazole buffer, a 1,2-dimethylimidazole buffer, a TAPS buffer, a bis-Tris buffer, a L-arginine buffer, a lactate buffer, a glycolate buffer, or combinations thereof.

In some aspects, a composition disclosed herein further comprises a bulking agent. Bulking agents can be added to a pharmaceutical product in order to add volume and mass to the product, thereby facilitating precise metering and handling thereof. Bulking agents that can be used with the present disclosure include, but are not limited to, sodium chloride (NaCl), mannitol, glycine, alanine, or combinations thereof.

In some aspects, a composition disclosed herein can also comprise a stabilizing agent. Non-limiting examples of stabilizing agents that can be used with the present disclosure include: sucrose, trehalose, raffinose, arginine, or combinations thereof.

In some aspects, a composition disclosed herein comprises a surfactant. In certain aspects, the surfactant can be selected from the following: alkyl ethoxylate, nonylphenol ethoxylate, amine ethoxylate, polyethylene oxide, polypropylene oxide, fatty alcohols such as cetyl alcohol or oleyl alcohol, cocamide MEA, cocamide DEA, polysorbates, dodecyl dimethylamine oxide, or combinations thereof. In some aspects, the surfactant is polysorbate 20 or polysorbate 80.

In some aspects, a composition comprising an IL-7 protein can be formulated using the same formulation used to formulate an additional agent disclosed herein (e.g., G-CSF and/or AMD3100). In other aspects, an IL-7 protein and an additional agent (e.g., G-CSF and/or AMD3100) are formulated using different formulations.

In some aspects, an IL-7 protein disclosed herein is formulated in a composition comprising (a) a basal buffer, (b) a sugar, and (c) a surfactant. In certain aspects, the basal buffer comprises histidine-acetate or sodium citrate. In some aspects, the basal buffer is at a concentration of about 10 to about 50 nM. In some aspects, a sugar comprises sucrose, trehalose, dextrose, or combinations thereof. In some aspects, the sugar is present at a concentration of about 2.5 to about 5.0 w/v %. In further aspects, the surfactant is selected from polysorbate, polyoxyethylene alkyl ether, polyoxyethylene stearate, alkyl sulfates, polyvinyl pyridone, poloxamer, or combinations thereof. In some embodiments, the surfactant is at a concentration of about 0.05% to about 6.0 w/v %.

In some aspects, the composition in which IL-7 is formulated further comprises an amino acid. In certain embodiments, the amino acid is selected from arginine, glutamate, glycine, histidine, or combinations thereof. In certain aspects, the composition further comprises a sugar alcohol. Non-limiting examples of sugar alcohol includes: sorbitol, xylitol, maltitol, mannitol, or combinations thereof.

In some aspects, an IL-7 protein disclosed herein is formulated in a composition comprising the following: (a) sodium citrate (e.g., about 20 mM), (b) sucrose (e.g., about 5%), (c) sorbitol (e.g., about 1.5%), and (d) Tween 80 (e.g., about 0.05%).

In some aspects, an IL-7 protein of the present disclosure is formulated as described in WO 2017/078385 A1, which is incorporated herein in its entirety.

A pharmaceutical composition (e.g., comprising an IL-7 protein disclosed herein) can be formulated for any route of administration to a subject. Specific examples of routes of administration include intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally. Parenteral administration, characterized by either subcutaneous, intramuscular or intravenous injection, is also contemplated herein. As described herein, in some aspects, an IL-7 protein can be administered in combination with an additional agent disclosed herein (e.g., G-CSF and/or AMD3100). In such aspects, the IL-7 protein and the additional agent can be administered using the same route of administration. In some aspects, an IL-7 protein and an additional agent are administered using different routes of administration.

Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Topical mixtures comprising an antibody are prepared as described for the local and systemic administration. The resulting mixture can be a solution, suspension, emulsions or the like and can be formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

A therapeutic agent described herein (e.g., an IL-7 protein alone or in combination with an additional agent disclosed herein, e.g., G-CSF and/or AMD3100) can be formulated as an aerosol for topical application, such as by inhalation (see, e.g., U.S. Pat. Nos. 4,044,126, 4,414,209 and 4,364,923, which describe aerosols for delivery of a steroid useful for treatment of inflammatory diseases, particularly asthma). These formulations for administration to the respiratory tract can be in the form of an aerosol or solution for a nebulizer, or as a microtine powder for insufflations, alone or in combination with an inert carrier such as lactose. In such a case, the particles of the formulation will, in one aspect, have diameters of less than 50 microns, in one aspect less than 10 microns.

A therapeutic agent disclosed herein (e.g., an IL-7 protein alone or in combination with an additional agent disclosed herein, e.g., G-CSF and/or AMD3100) can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the antibody alone or in combination with other pharmaceutically acceptable excipients can also be administered.

Transdermal patches, including iontophoretic and electrophoretic devices, are well known to those of skill in the art, and can be used to administer an antibody. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957, each of which is herein incorporated by reference in its entirety.

In certain aspects, a pharmaceutical composition comprising a therapeutic agent described herein (e.g., an IL-7 protein alone or in combination with an additional agent disclosed herein, e.g., G-CSF and/or AMD3100) is a lyophilized powder, which can be reconstituted for administration as solutions, emulsions and other mixtures. It can also be reconstituted and formulated as solids or gels. The lyophilized powder is prepared by dissolving an antibody or antigen-binding portion thereof described herein, or a pharmaceutically acceptable derivative thereof, in a suitable solvent. In some aspects, the lyophilized powder is sterile. The solvent can contain an excipient which improves the stability or other pharmacological component of the powder or reconstituted solution, prepared from the powder. Excipients that can be used include, but are not limited to, dextrose, sorbitol, fructose, corn syrup, xylitol, glycerin, glucose, sucrose or other suitable agent. The solvent can also contain a buffer, such as citrate, sodium or potassium phosphate or other such buffer known to those of skill in the art at, in one aspect, about neutral pH. Subsequent sterile filtration of the solution followed by lyophilization under standard conditions known to those of skill in the art provides the desired formulation. In some aspects, the resulting solution can be apportioned into vials for lyophilization. Each vial can contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier.

The compositions to be used for in vivo administration can be sterile. This can be accomplished by filtration through, e.g., sterile filtration membranes.

All publications, patents, patent applications and other documents cited in this application are hereby incorporated by reference in their entireties for all purposes to the same extent as if each individual publication, patent, patent application or other document were individually indicated to be incorporated by reference for all purposes.

The following examples are merely illustrative and should not be construed as limiting the scope of this disclosure in any way as many variations and equivalents will become apparent to those skilled in the art upon reading the present disclosure.

### Examples

### Example 1: Materials and Methods

In carrying out the Examples described below, one or more of the following materials and methods were used.

### Animal Studies

C57BL/6, CD45.1 (#002014), RAG-1 KO (#002216), Mb-1^{Cre} (#020505), and IL-7R^{flox/flox} (#022143) female or male mice (6-12 weeks old) were purchased from Jackson Laboratories and maintained in a specific pathogen-free animal facility at POSTECH.

### Human Sample Analysis

Human PBMC samples were obtained from healthy volunteers after IL-7 protein (60 ug/kg) subcutaneously treatment (NCT02860715) using a density gradient separation with Ficoll-Paque PLUS (GE Healthcare). Absolute CD34⁺ cell count was calculated by multiplying the number of PBMCs determined by an automated hematology analyzer with the percentage of CD34⁺ cells enumerated by flow cytometry analysis.

### Cell Preparation

Mice were anesthetized by intraperitoneal injection of ketamine (Yuhan Co.) and xylazine (Bayer) and blood was obtained by cardiac puncture. PBMCs were obtained using a density gradient separation with HISTOPAQUE®-1083 (Sigma, 10831). BM cells were harvested by flushing femur and tibia with RPMI 1640 medium (WELGENE, LM 011-01) containing newborn calf serum (Gibco, 26010074). Absolute cell numbers were counted with an automated Vi-CELL XR analyzer (Beckman Coulter).

### Transplantation

For evaluating the multi-potency of mobilized HSCs, lethally irradiated (9Gy) CD45.1⁺ recipients were transplanted with 2×10⁶ PBMCs isolated from IL-7 treated (2.5 mg/kg) CD45.2⁺ mice at day 3 post-treatment or freshly harvested control BM cells. Blood chimerism was analyzed 8 weeks after reconstitution.

For serial transplantation, CD45.2⁺ donor mice were treated control or IL-7 protein (2.5 mg/kg) and PBMCs were isolated at day 3 post-treatment. 2×10⁶ PBMCs were intravenously injected into lethally irradiated CD45.1⁺ primary recipients. After the immune reconstitution period for 16 weeks, secondary or tertiary recipients were transplanted with primary or secondary freshly harvested BM cells, respectively.

For competitive blood stem cell transplantation, CD45.2⁺ mice were treated IL-7 protein (2.5 mg/kg) or PEG-rhG-CSF 5 μg and PBMCs were isolated at day 3 post-treatment. 2×10⁶ PBMCs were intravenously injected into lethally irradiated CD45.1⁺/CD45.2⁺ recipients with freshly harvested competitor BM cells (CD45.1⁺, 0.5×10⁶ cells). Blood chimerism was analyzed 8, 12, and 18 weeks after reconstitution.

### Flow Cytometry

To analyze human CD34⁺ cells in the blood, CD34 (clone 8G12), CD45 (clone 2D1), and 7-amino-actinomycin D (7-AAD) were used according to recommendations of the ISHAGE guidelines¹. For analysis of mouse HSCs and progenitors, the following monoclonal antibodies were used: lineage cocktail (TER119 (clone TER-119), CD11b (clone M1/70), CD3ε (clone 145-2C11), B220 (clone RA3-6B2), CD19 (HIB19), NK1.1 (clone PK136), Gr-1 (clone RB6-8C5), and MHCII (I-A/I-E, clone M5/114.15.2)), c-Kit (clone 2B8), Sca-1 (clone D7), CD150 (clone mShad150), and CD48 (clone HM48-1). For analysis of mouse B cell subsets, B220, CD43 (clone S11), CD24 (clone M1/69), IgM (clone eB121-15F9), and IgD (clone 11-26c.2a) were used. For the mechanistic study, CD127 (clone A7R34), CD45 (clone 30-F11), CXCR4 (clone L276F12), and VLA-4 (clone R1-2) were additionally used. All samples were assessed on LSRFortessa or FACSCanto (BD) and analyzed using FlowJo software. Cell sorting was performed on MoFlo XDP (Beckman Coulter).

### Quantitative Real-Time PCR

BM CD45⁻TER119⁻7-AAD⁻ cells were sorted and RNA was extracted using a TRIzol (Invitrogen). After genomic DNA elimination, cDNA was synthesized with a QuantiTect® Reverse Transcription Kit (Qiagen). Using Power SYBR Green PCR master mix (ThermoFisher scientific), real-time PCR was carried out on ViiA 7 Real-Time PCR system (ThermoFisher scientific). The relative expression of target genes was normalized to Rpl32 (L32). The primers were used as follows: Rpl32 (forward: 5′-GAA ACT GGC GGA AAC CCA-3′ (SEQ ID NO: 73), reverse: 5′-TCT GGC CCT TGA ACC TT-3′ (SEQ ID NO: 74)); Cxcl12 (forward: 5′-TTT CAG ATG CTT GAC GTT GG-3′ (SEQ ID NO: 75), reverse: 5′-GCG CTC TGC ATC AGT GAC-3′ (SEQ ID NO: 76)); Kitl (also known as SCF) (forward: 5′-CTC TTC AAC ATT AGG TCC CGA GAA AGG GAA AG-3′ (SEQ ID NO: 77), reverse: 5′-CTT CCA GTA TAA GGC TCC AAA AGC AAA GCC A-3′ (SEQ ID NO: 78)); and Vcam1 (forward: 5′-TCG GGC GAA AAA TAG TCC TT-3′ (SEQ ID NO: 79), reverse: 5′-CCG GCA TAT ACG AGT GTG AA-3′ (SEQ ID NO: 80)). All procedures were performed according to the manufacturers' protocols.

### Statistical Analysis

Data were analyzed using GraphPad Prism version 8.4.1 for Windows (GraphPad Software, San Diego, California USA) and were presented as mean±SEM. Statistical significance was determined by unpaired two-tailed t-test for the comparison of two groups, Kruskal-Wallis test or one-way ANOVA with Bonferroni's post hoc test for multiple comparisons, Log-rank test for survival comparison, or Spearman's correlation coefficient analysis for the correlation between BM proB cells and PB HSCs. P values<0.05 were considered to be significant.

### Example 2: Effect of IL-7 Administration on Hematopoietic Stem Cell Mobilization in Mice

To begin assessing the potential role of IL-7 on hematopoietic stem cell (HSC) mobilization from the bone marrow to the peripheral blood, a single dose of an IL-7 protein disclosed herein (2.5 mg/kg) was subcutaneously administered to C57BL/6 mice (6-12 weeks; Jackson Laboratories). Then, at various time points post IL-7 administration, the presence of different cell populations within the peripheral blood and bone marrow was determined using flow cytometry.

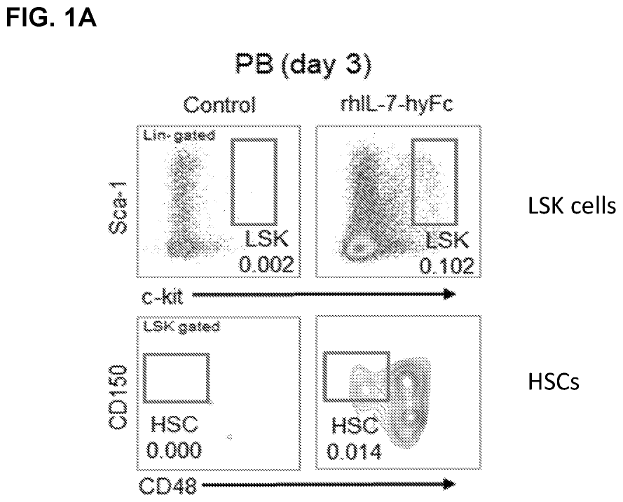

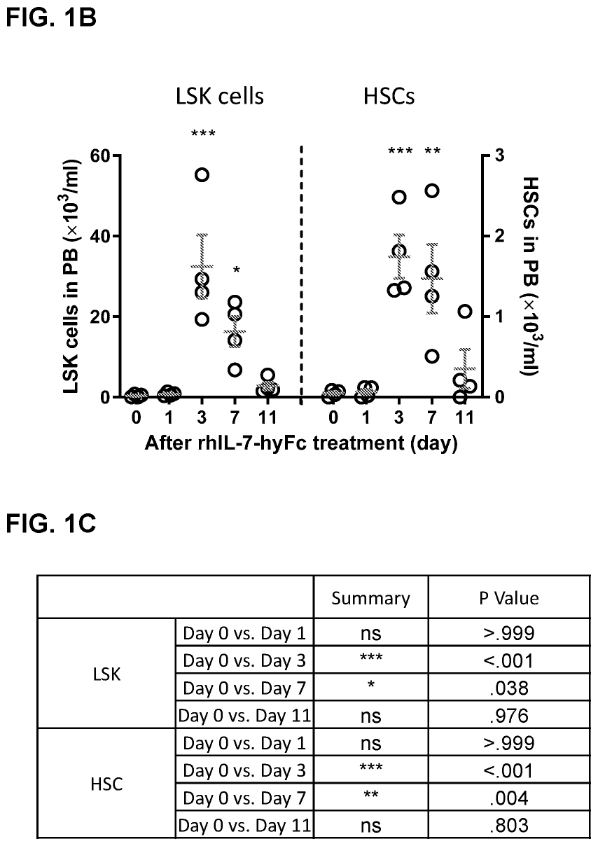

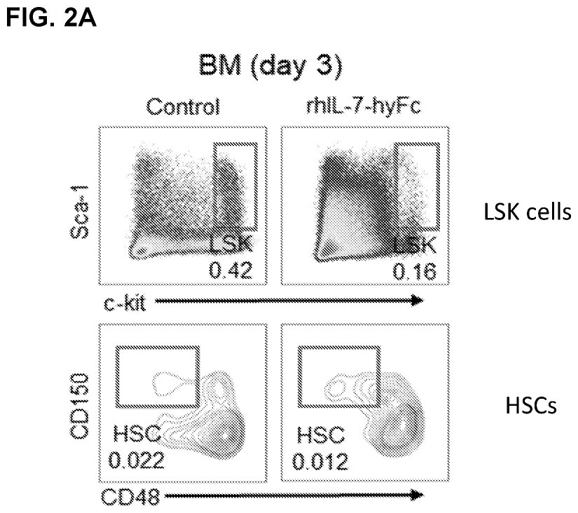

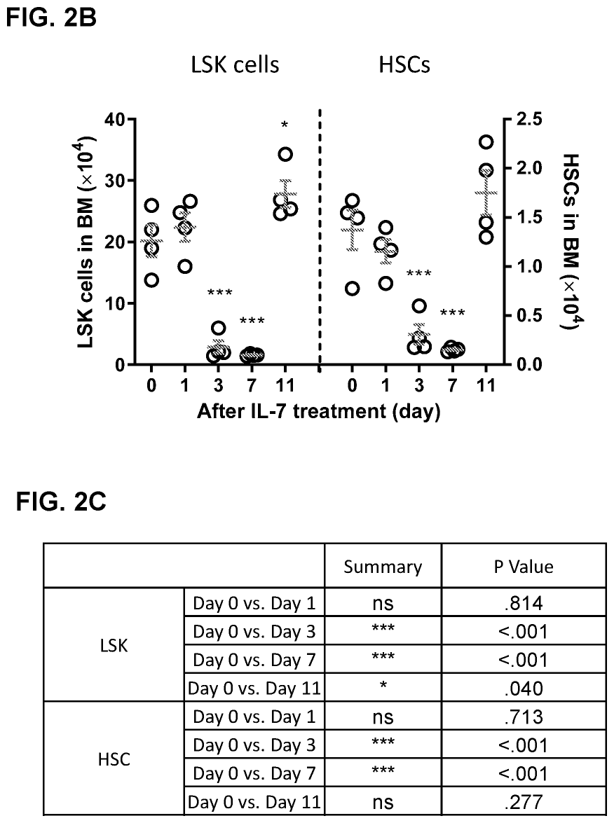

As shown in FIGS. 1A and 2A, at day 3 post-administration, there was a significant increase in the number of circulating total hematopoietic stem and progenitor (Lin⁻ Sca-1⁺ c-kit⁺; “LSK”) cells in the peripheral blood with a corresponding decrease in the bone marrow. Similar results were observed when hematopoietic stem cells (HSCs) (i.e., LSK cell subset) were specifically analyzed. This data indicated that administering the IL-7 protein resulted in the mobilization of HSCs and progenitors from the bone marrow to the periphery. And, as shown in FIGS. 1B, 1C, 2B, and 2C, the effect of IL-7 on HSC mobilization peaked at around day 3 post-administration and lasted for at least 7 days after administration.

### Example 3: Dose-Dependent Effect of IL-7 Administration on Hematopoietic Stem Cell Mobilization in Mice

Next, to determine whether the effect observed in Example 2 was dependent on the dose of the IL-7 protein, C57BL/6 mice received a single administration of the IL-7 protein at one of the following doses: (i) 0 mg/kg, (ii) 0.1 mg/kg, (iii) 0.5 mg/kg, (iv) 2.5 mg/kg, or (v) 12.5 mg/kg. Then, at day 3 post-administration, the number of LSK cells and HSCs were determined in the peripheral blood (as described in Example 2).

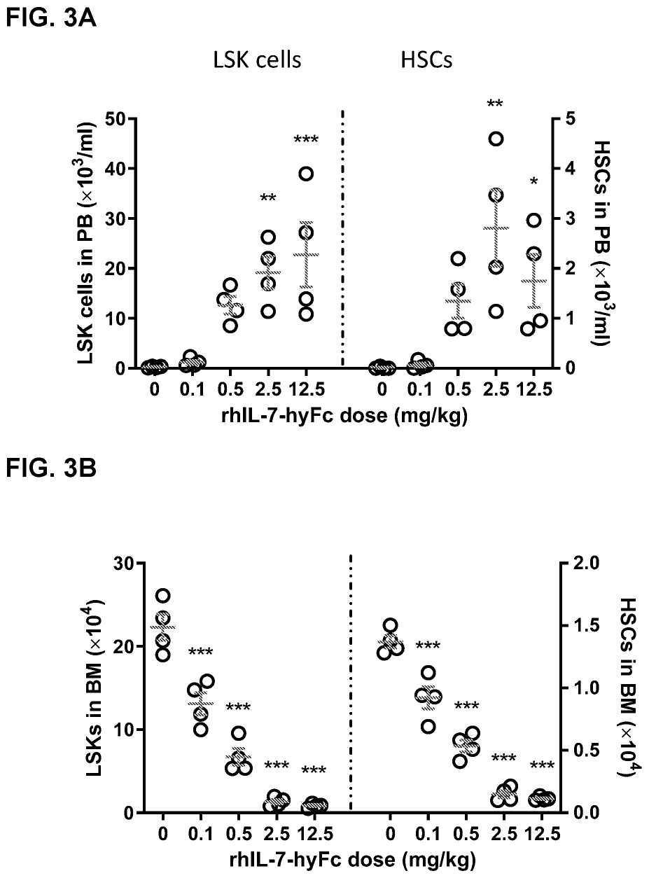

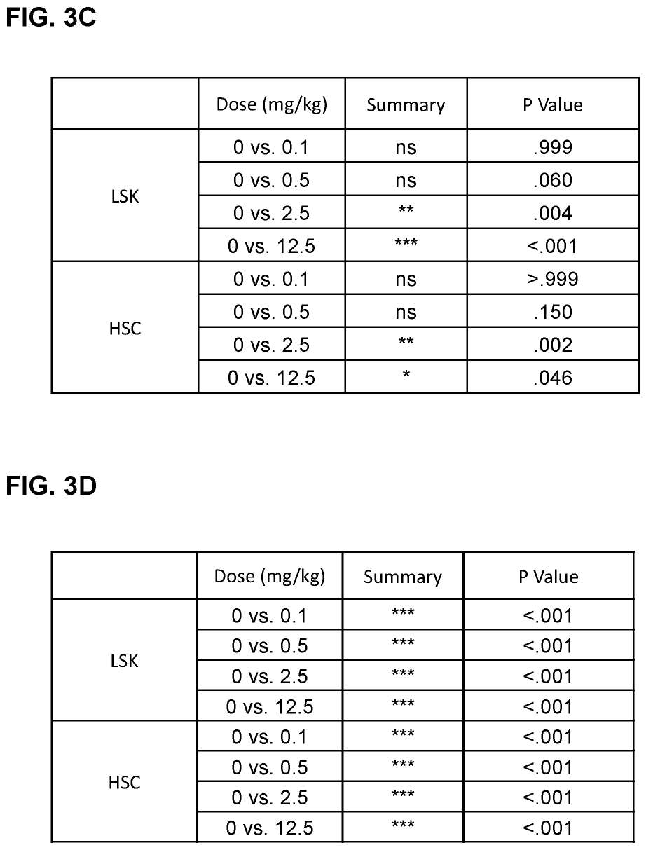

As shown in FIGS. 3A and 3C, there was a dose-dependent increase in the number of both LSK cells and HSCs in the peripheral blood of the IL-7 treated animals. As observed in Example 2, there was a corresponding decrease in the number of both LSK cells and HSCs in the bone marrow (see FIGS. 3B and 3D). Optimum dose of the IL-7 protein appeared to be 2.5 mg/kg, as there did not appear to be a significant difference in effect at 2.5 mg/kg compared to at 12.5 mg/kg.

### Example 4: Effect of IL-7 Administration on the Mobilization of Different Hematopoietic Stem and Progenitor (LSK) Cell Subsets in Mice

As described herein, hematopoietic stem and progenitor (LSK) cells are considered to be a heterogeneous population made up of different subsets. Ema et al., Exp Hematol 42(2):74-82 (February 2014), which is incorporated herein by reference in its entirety. To determine whether IL-7 administration can promote the mobilization of different LSK subsets, C57BL/6 mice were again treated with a single dose of the IL-7 protein (2.5 mg/kg). Control mice were treated with a control buffer. Then, at day 3 post-administration, the number of different LSK subsets in both the peripheral blood and bone marrow was determined.

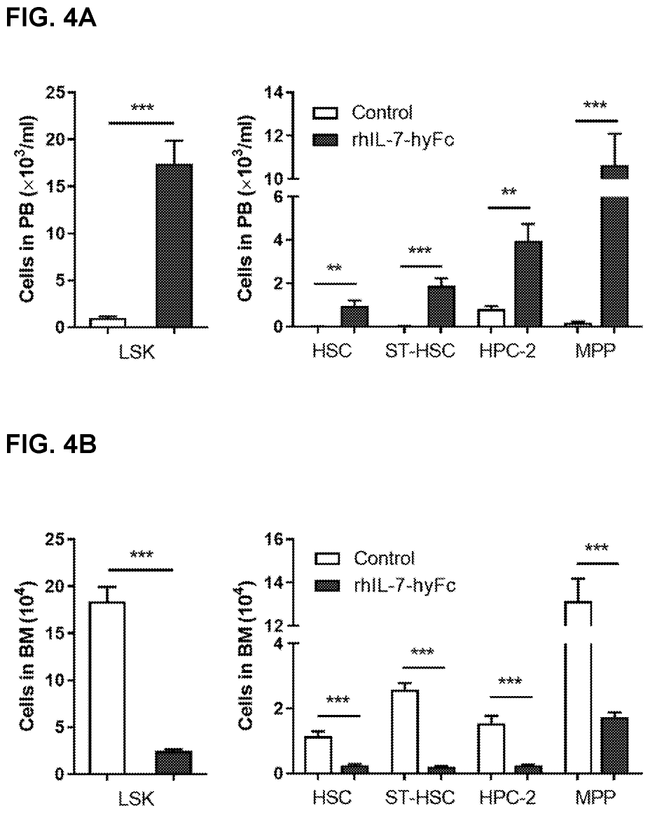

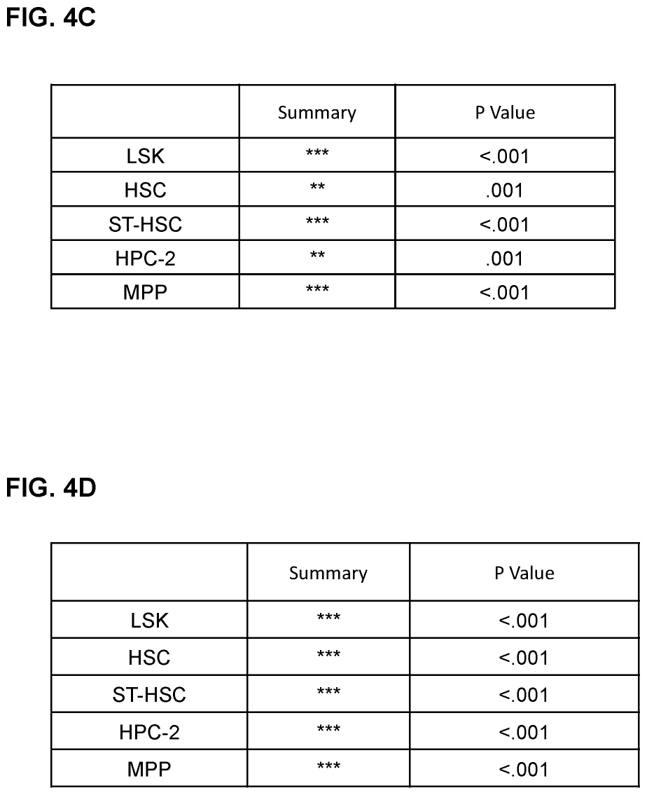

As shown in FIGS. 4A-4D, IL-7 administration resulted in the mobilization of all LSK subsets analyzed, i.e., hematopoietic stem cells (HSCs), short-term HSCs (“ST-HSC”), multipotent progenitors (“MPP”), and hematopoietic progenitor cells-2 (“HPC-2”), from the bone marrow to the peripheral blood.

Collectively, the results shown in Examples 2-4 demonstrate the potency of IL-7 in promoting the mobilization of LSKs (e.g., HSCs) to the peripheral blood.

### Example 5: Functional Analysis of HSCs after IL-7 Administration

Because transplanted HSCs into patients have to be capable of replenishing the whole-blood system through a lifetime, maintenances of stem cell function with multi-potency and long-term reconstituting capacity are critical for clinical outcomes. Therefore, the in vivo functionality of the HSCs mobilized with IL-7 protein was assessed as described below.

To assess the multi-potency of the mobilized HSCs, a stem cell transplant assay was performed using (i) peripheral blood mononuclear cells (PBMCs) isolated from mice treated with the IL-7 protein and (ii) freshly harvested bone marrow from control mice (i.e., untreated mice). The isolated cells were then transplanted into lethally irradiated recipient mice. At 8 weeks post-transplantation, the hematopoietic lineage production was assessed by determining the percentage of myeloid cells, T cells, and B cells present in the peripheral blood of the different recipient animals.

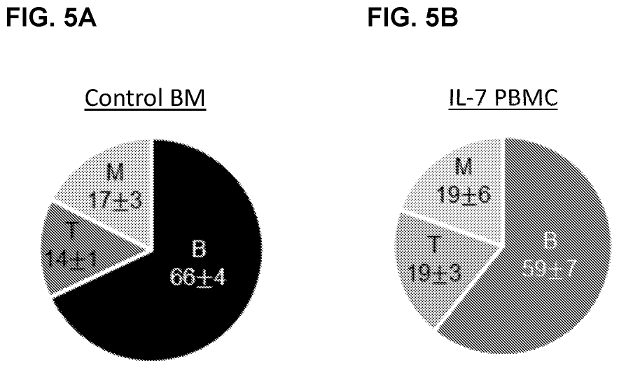

As shown in FIGS. 5A and 5B, myeloid (CD11b⁺) and lymphoid (B220⁺ and CD3e⁺) (i.e., B and T cells, respectively) lineage production derived from PBMCs (isolated from IL-7 treated donor animals) were comparable to those derived from control bone marrow cells. These results suggest that the IL-7-induced mobilized HSCs are multi-potent without any differentiation skewing toward specific lineages, as compared to normal bone marrow HSCs.

Next, to determine the long-term reconstituting capacity of the mobilized HSCs, a serial transplantation assay was performed using PBMCs isolated from mice treated with IL-7 protein or a control buffer. The isolated PBMCs were transplanted into lethally irradiated recipient mice, and then the survival of the mice was assessed. For the secondary and tertiary transfer, bone marrow cells from the surviving mice after the primary and secondary transfer, respectively, were transferred into new irradiated recipient animals.

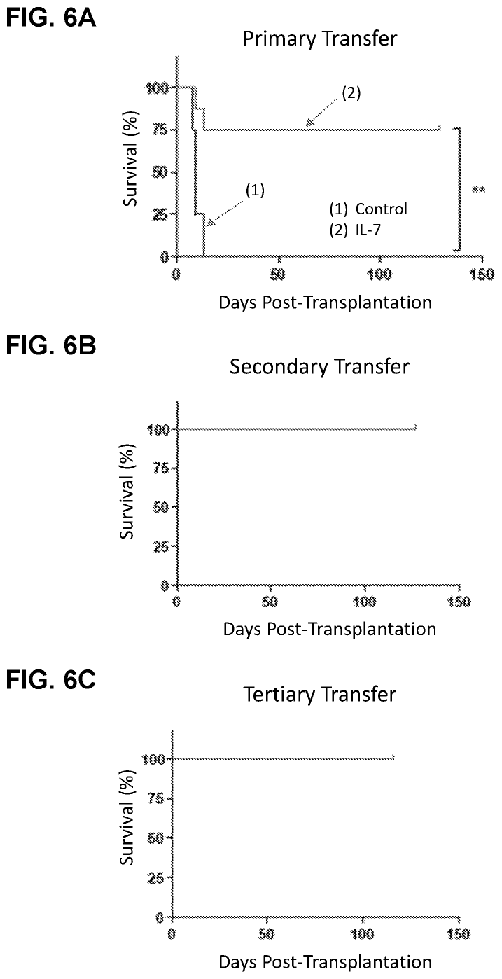

As shown in FIG. 6A, 75% of the recipient mice transplanted with PBMCs from IL-7 treated animals survived the entire duration of the experiment (approximately 16 weeks post transplantation). In contrast, recipient mice transplanted with PBMCs from the control animals had all died within 2 weeks post-transplantation. When the bone marrow cells from the surviving mice (i.e., transplanted with PBMCs from IL-7 treated animals) were transplanted into new recipients for the secondary and tertiary transfers, all the recipient mice survived (see FIGS. 6B and 6C), demonstrating that the HSCs mobilized with IL-7 are capable of sustaining hematopoietic reconstitution long-term.

The above results demonstrate that the HSCs mobilized with a single dose of IL-7 administration are functionally similar to normal bone marrow HSCs and have long-term multi-lineage reconstituting capability.

### Example 6: Effect of IL-7 Administration on the Mobilization of HSCs in Humans

To confirm whether IL-7 can also promote the mobilization of HSCs to peripheral blood in humans, healthy human volunteers were given a single dose of either placebo or an IL-7 protein (60 μg/kg; via subcutaneous administration) (ClinicalTrials.gov Identifier: NCT02860715). Then, the number of HSCs (i.e., CD34+ cells in humans) in the peripheral blood was assessed using flow cytometry at 10 days post administration. For comparison purposes, the number of HSCs prior to the administration (i.e., day 0) was also assessed in the healthy human volunteers.

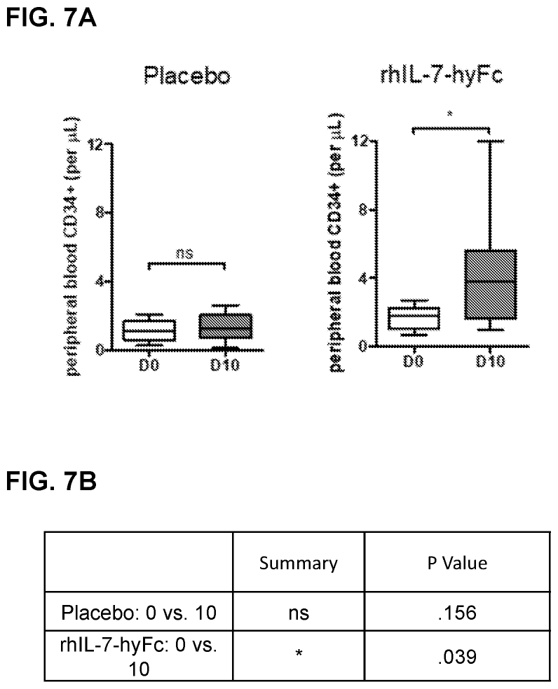

As shown in FIGS. 7A and 7B, human subjects that received the placebo did not have any noticeable change in the number of HSCs observed in the peripheral blood. In contrast (and in agreement with the animal data provided above—see, e.g., Example 2), in many of the human subjects that received an administration of the IL-7 protein, there was a significant increase in the number of HSCs observed in the peripheral blood at day 10 post IL-7 administration.

The above results demonstrate that the IL-7 protein disclosed herein could also be effective in promoting the mobilization of HSCs into peripheral blood in human subjects.

### Example 7: Role of ProB Cells on the Mobilization of HSCs Induced by IL-7

To begin identifying possible mechanisms by which IL-7 induces the mobilization of HSCs to the peripheral blood, a recombination-activating gene 1-deficient mice (“RAG-1 KO”) were used. These mice fail to produce mature lymphocytes from preB cells in the bone marrow and from preT cells in the thymus. Briefly, normal (wild-type) and RAG-1 KO mice were treated with either a single dose of IL-7 protein (2.5 mg/kg) (black bars) or control buffer (white bars). Then, at day 3 post administration, the number of LSK cells and HSCs were determined in the peripheral blood of the different animals.

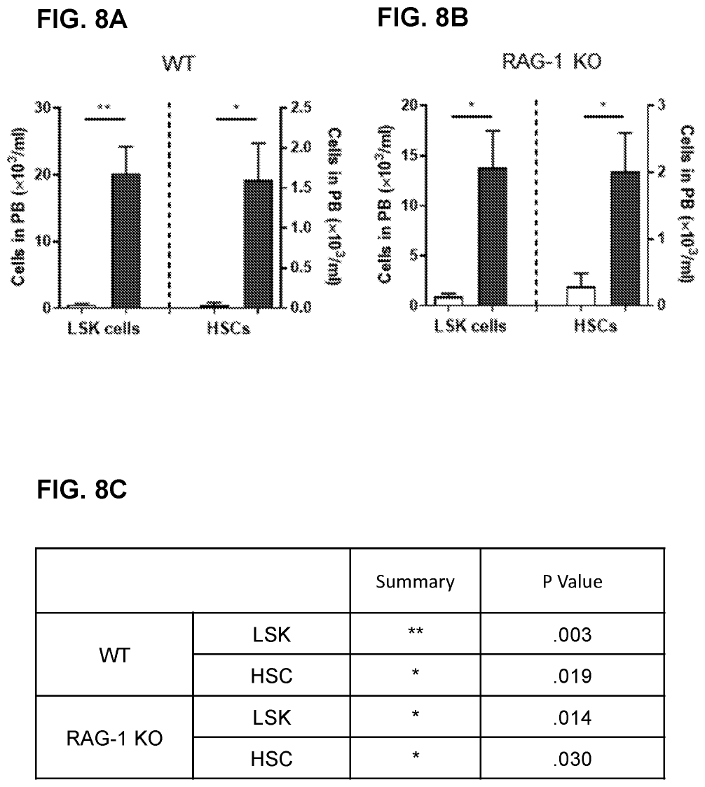

In agreement with the earlier examples (see, e.g., Examples 2-4), the administration of IL-7 protein resulted in the mobilization of both LSK cells and HSCs to the peripheral blood in wild-type animals (see FIG. 8A). As shown in FIG. 8B, similar results were observed in the RAG-1 KO mice. The observed effect of the IL-7 protein was statistically significant as shown in FIG. 8C. The results demonstrate that mature T cells and developing pre/immature B cells are not required for IL-7-induced mobilization of HSCs, as well as other LSK cell subsets.

RAG-1 KO mice still have early-stage developing B cells prior to preB cells in the bone marrow. Therefore, any potential alteration in the development of B cell subsets was assessed in the bone marrow of mice treated with either the control buffer or IL-7 protein using flow cytometry.

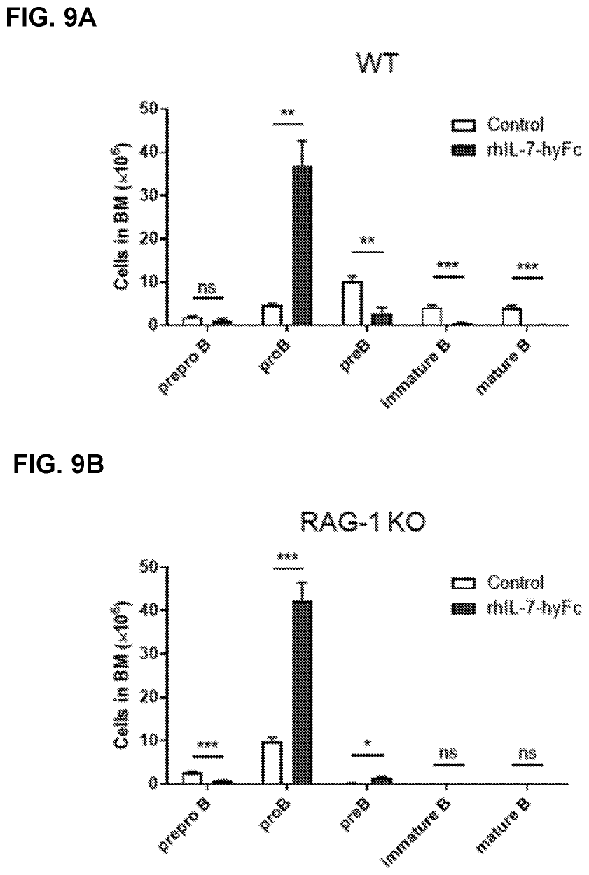

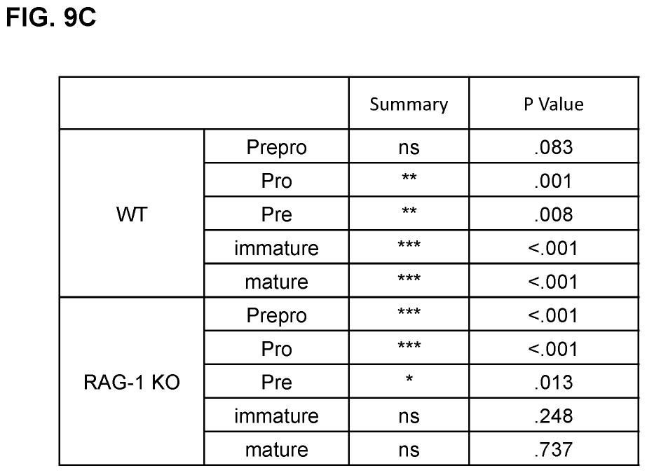

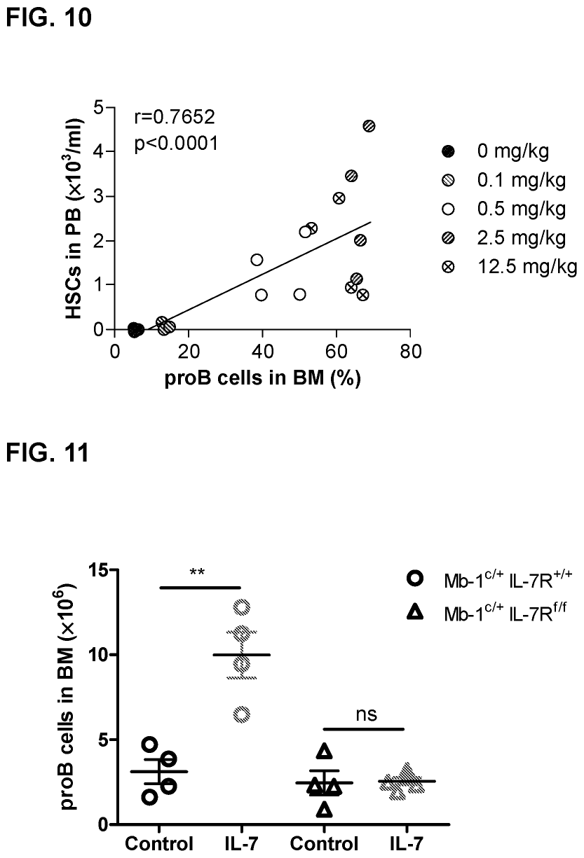

As shown in FIGS. 9A-9C, no major differences were observed for pre-proB cells, but there was a significant increase in the number of proB cells in both wild-type and RAG-1 KO mice treated with the IL-7 protein, compared to those treated with the control buffer. Furthermore, in response to the IL-7 protein, there was a strong correlation between the increased number of bone marrow proB cells and the number of mobilized HSCs in the peripheral blood (see FIG. 10).

To further assess the potential role of proB cells in the mobilization of HSCs induced by IL-7, a proB-specific IL-7R deficient mice (“Mb-1^{cre/+}IL-7R^{flox/flox}”) was generated. Again, either the IL-7 protein (2.5 mg/kg) or control buffer was administered to the proB-specific IL-7R deficient mice. As a control, IL-7 protein (2.5 mg/kg) and control buffer were also administered to normal control (“Mb-1^{cre/+}IL-7R^{+/+}”). Then, at day 3 post administration, the number of proB cells was determined in the bone marrow of the different animals. The number of LSK cells and HSCs both in the peripheral blood and the bone marrow were also assessed.

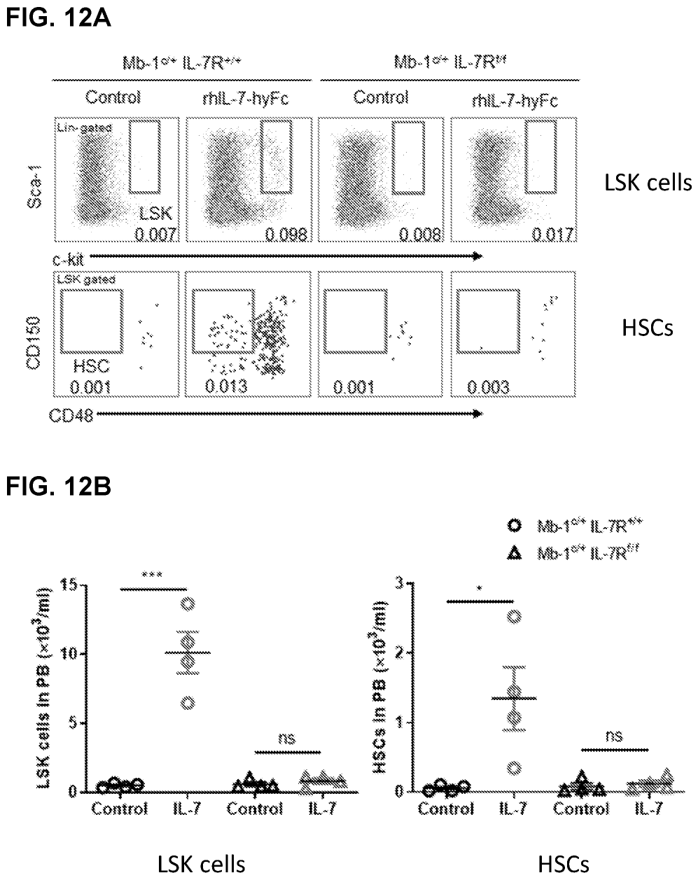

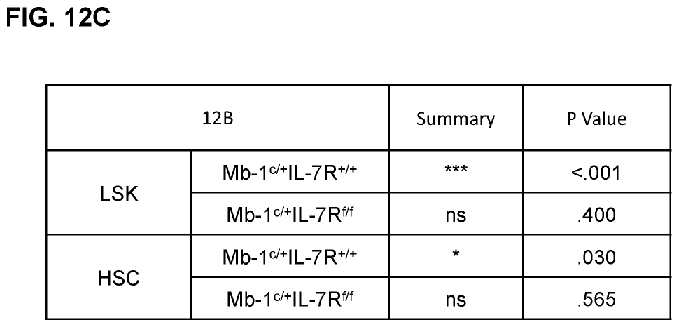

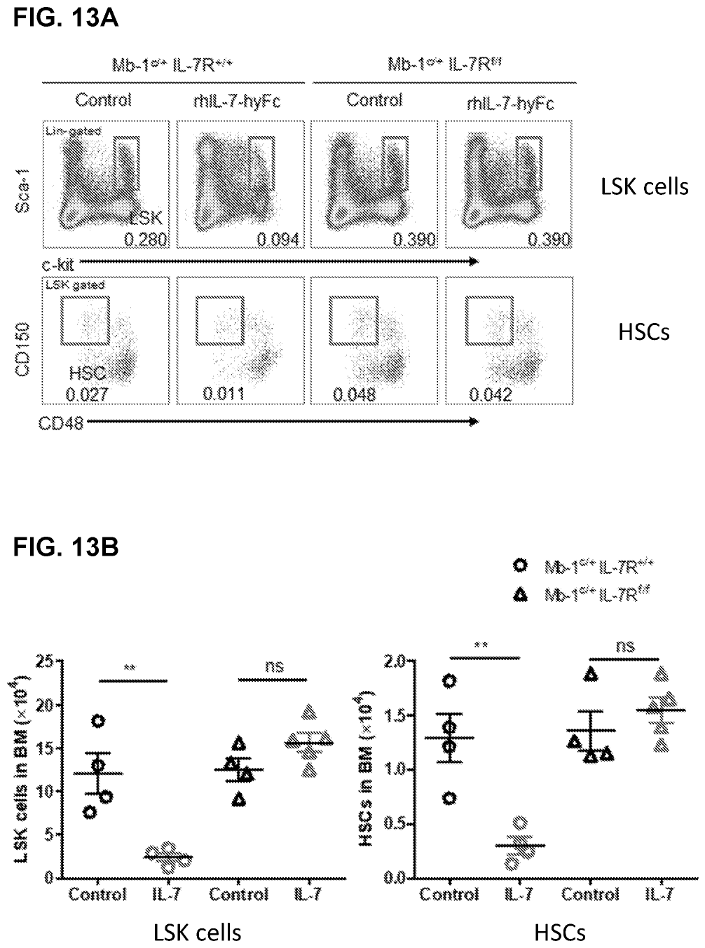

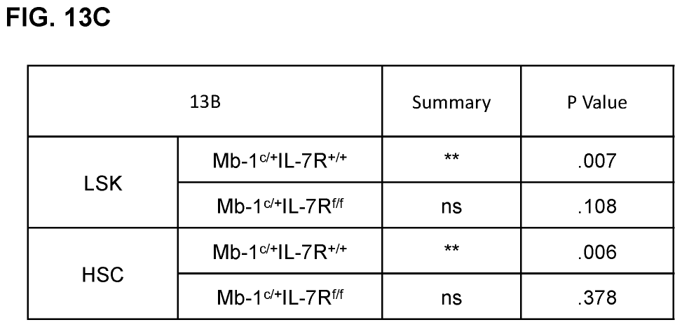

As shown in FIG. 11, unlike the control mice, there was not a significant difference in the number of proB cells in the bone marrow of proB-specific IL-7R deficient mice treated with the IL-7 protein compared to the corresponding mice treated with the control buffer. The lack of increase in the number of proB cells correlated with lack of HSC mobilization (see FIGS. 12A-12C and 13A-13C).

Not to be bound by any one theory, the above results collectively suggest that bone marrow proB cells could play a role in the mobilization of HSCs, as well as other LSK cells, to the peripheral blood after IL-7 administration.

### Example 8: Role of Niche Factors on the Mobilization of HSCs Induced by IL-7

It has previously been suggested that blocking niche interactions between HSCs and their surrounding niche cells can induce the mobilization of HSCs. Therefore, the potential role that alterations of niche factors could play in IL-7-induced mobilization of HSCs was next assessed. Briefly, normal C57BL/6 mice were treated with either the control buffer or IL-7 protein (2.5 mg/kg). Then, at day 2 post administration, the expression of different niche factors was assessed in the bone marrow.

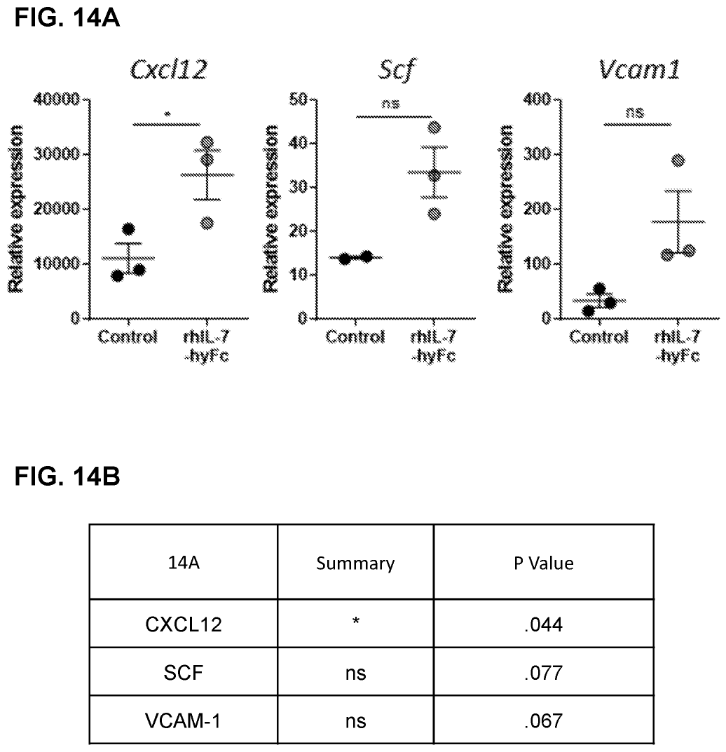

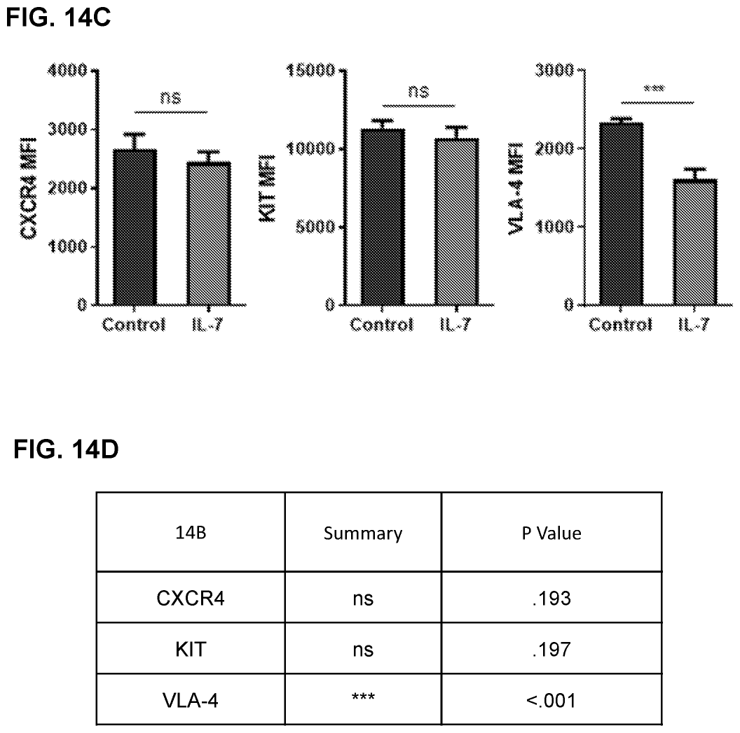

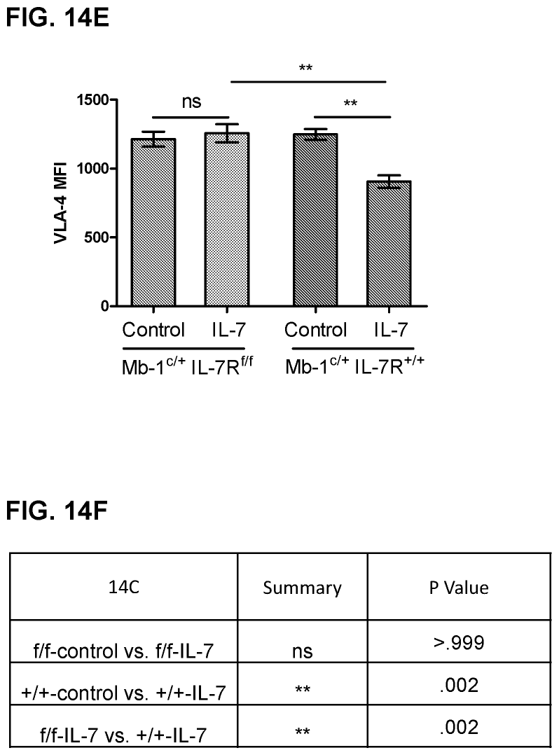

As shown in FIGS. 14A and 14B, the administration of IL-7 protein did not show significant decreases in retention-related gene expressions on non-hematopoietic niche cells, such as Cxcl12, Scf, and Vcam1. However, for retention signals expressed on HSCs, there was a noticeable decrease in the expression of VLA-4 (but not of CXCR4 and KIT) in the IL-7 treated mice compared to those treated with the control buffer (see FIGS. 14C and 14D). The reduction in VLA-4 expression appeared to be dependent on IL-7 receptor (IL-7R) expression in proB cells (see FIGS. 14E and 14F).

Not to be bound by any one theory, the above results suggest that the downregulation of VLA-4 through proB-dependent IL-7 signaling could play a role in mobilizing HSCs to peripheral blood after IL-7 protein administration.

### Example 9: Comparison of IL-7 to G-CSF on the Mobilization of HSCs

As described herein, granulocyte colony stimulating factor (G-CSF) is a standard regimen used in the clinic to induce HSC mobilization. Therefore, the potency of IL-7 was compared to that of G-CSF on inducing the mobilization of HSCs to peripheral blood was assessed. Briefly, C57BL/6 mice were treated with one of the following: (i) control buffer (“control”), (ii) IL-7 protein (“IL-7”) (2.5 mg/kg; subcutaneous), (iii) long-acting pegylated recombinant human G-CSF (“PEG-rhG-CSF”) (5 μg; subcutaneous), or (iv) non-pegylated recombinant human G-CSF (“rhG-CSF”) (twice a day (6.25 μg/day) at days 0, 1, 2, and 3 (i.e., total dose of 25 μg/mouse); subcutaneous). In animals treated with the IL-7 protein or PEG-rhG-CSF, the number of LSK cells and HSCs was determined in the peripheral blood at day 3 post administration. In animals treated with rhG-CSF, the number of LSK cells and HSCs in the peripheral blood was assessed within 6-8 hours after last rhG-CSF administration.

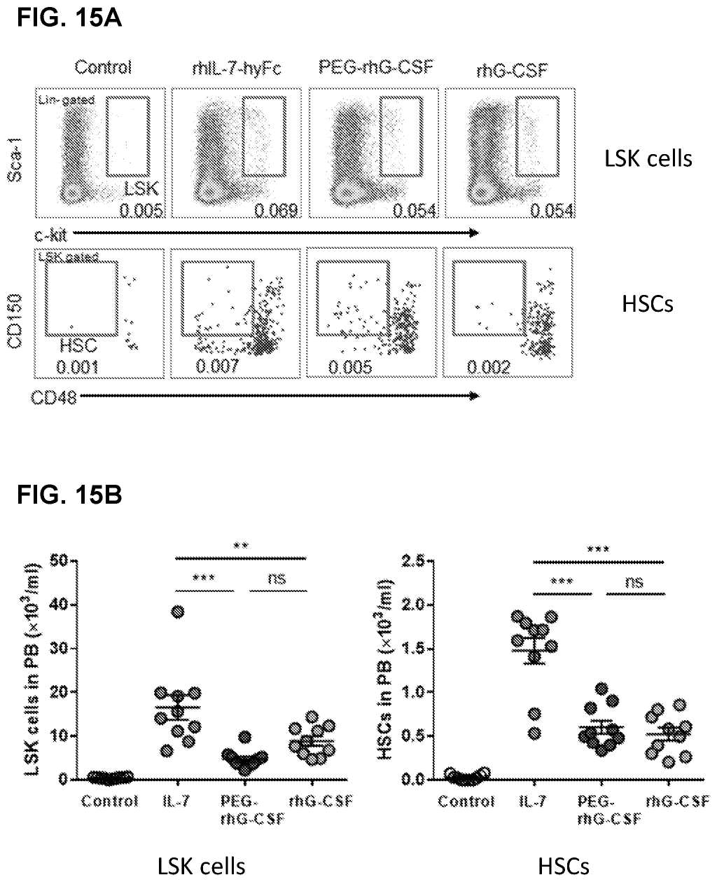

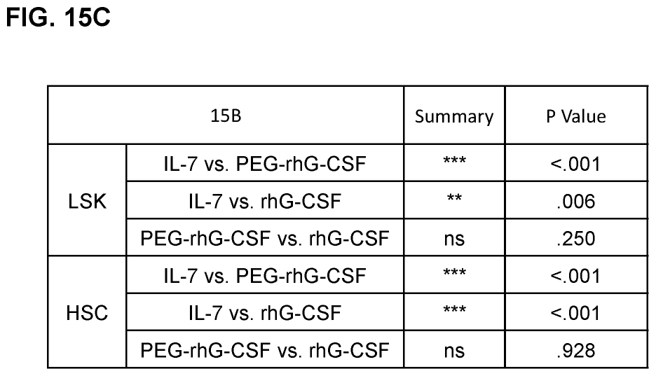

As shown in FIGS. 15A-15C, mice treated with the IL-7 protein had significantly greater number of both LSK cells and HSCs in the peripheral blood (approximately a 3-fold increase) compared to mice treated with PEG-rhG-CSF or rhG-CSF. Because the two types of G-CSF showed comparable efficacy of mobilization, the long-acting PEG-rhG-CSF was used in the subsequent experiments.

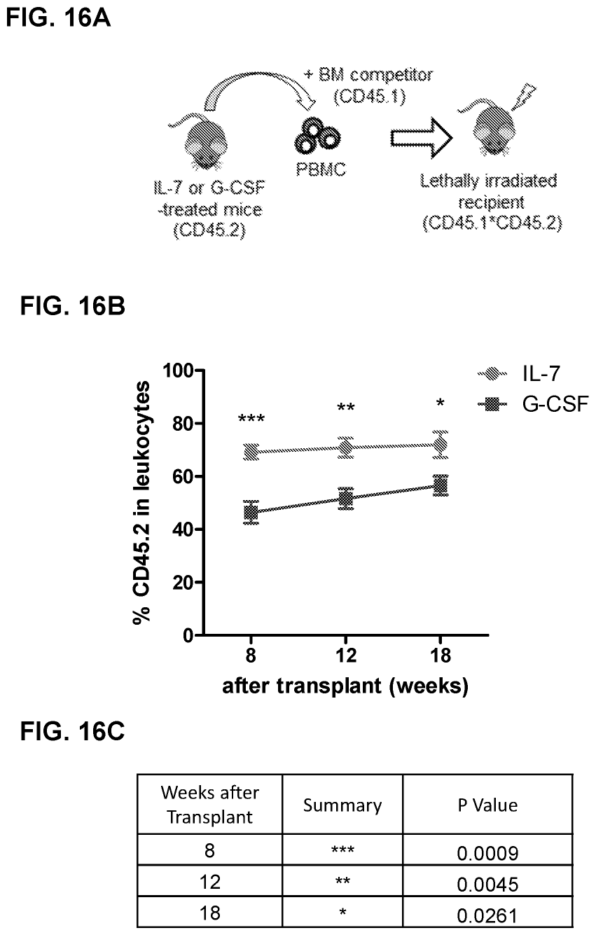

As further analysis, the reconstitution capacity of HSCs mobilized after IL-7 administration was compared to those mobilized after G-CSF administration using a competitive reconstitution assay shown in FIG. 16A. Briefly, CD45.2⁺ mice were treated with IL-7 protein (2.5 mg/kg) or G-CSF (5 μg), and PBMCs were isolated at day 3 post-treatment. PBMCs (2×10⁶ cells) were intravenously injected into lethally irradiated CD45.1⁺/CD45.2⁺ recipients with freshly harvested competitor BM cells (CD45.1⁺, 0.5×10⁶ cells). Blood chimerism was analyzed 8, 12, and 16 weeks after reconstitution.

As shown in FIGS. 16B and 16C, PBMCs isolated from IL-7 treated mice also showed higher reconstitution capacity compared with PBMCs isolated from G-CSF-treated mice. Similar to total leukocyte reconstitution (see FIGS. 16B and 16C), the lymphoid, i.e., B cells (B220⁺) and T cells (CD3e⁺), and myeloid (CD11b⁺) lineage reconstitution by PBMCs from the IL-7 treated animals also tended to be higher than that of the G-CSF treated group (see FIGS. 16D and 16E).

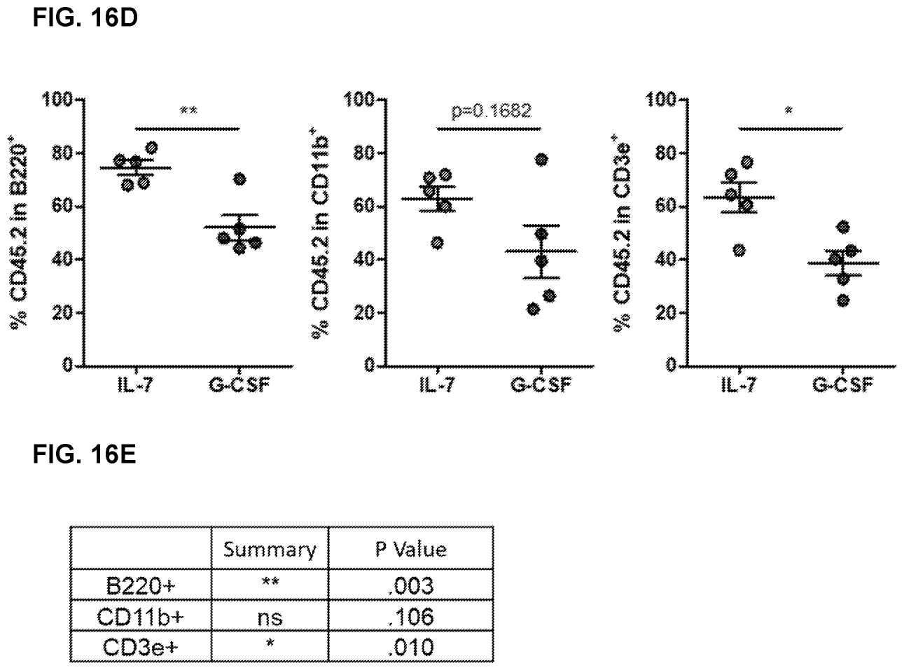

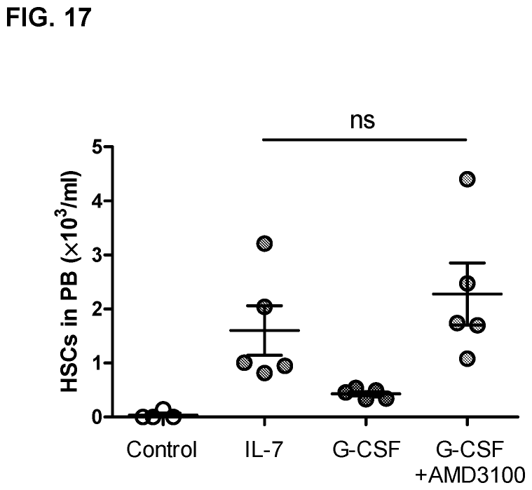

In the clinic, G-CSF is combined with AMD3100 (a CXCR4 antagonist) to improve the mobilization efficacy in patients who previously failed to mobilize sufficiently with G-CSF alone. In this regard, the potency of IL-7 protein alone to the G-CSF+AMD3100 combinational regimen was next compared. Briefly, normal C57BL/6 mice were administered with one of the following: (i) control buffer, (ii) IL-7 protein alone, (iii) pegylated recombinant human G-CSF (“G-CSF”), or (iv) G-CSF in combination with AMD3100. Then, the number of HSCs in the peripheral blood was assessed at day 3 post administration. As shown in FIG. 17, a single dose of IL-7 protein alone was just as effective as the combination of G-CSF and AMD3100 in inducing the mobilization of HSCs to the peripheral blood.

Taken together, the above results demonstrate that the IL-7 protein disclosed herein is capable of mobilizing HSCs with reconstitution capacity more effectively than G-CSF, a standard agent for mobilization in the clinic, and its mobilization capacity is sufficient to replace the combined therapy of G-CSF and AMD3100.

### Example 10: Effect of IL-7 and G-CSF Combination on HSC Mobilization into Peripheral Blood

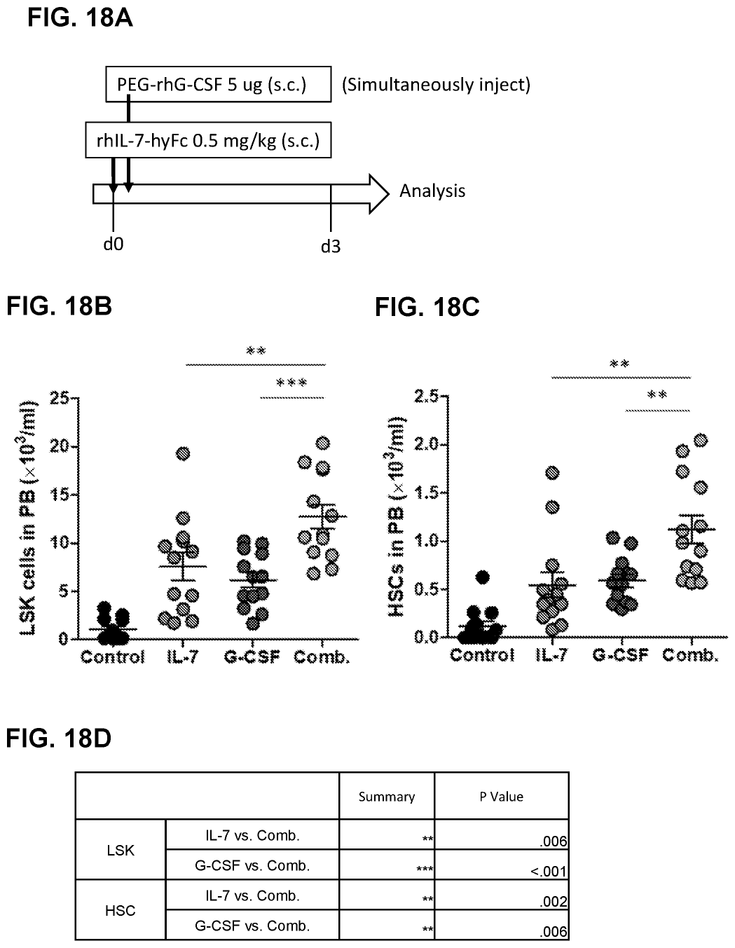

To determine whether the combined treatment of IL-7 and G-CSF can further improve the mobilization of HSCs into the peripheral blood, C57BL/6 animals received a single subcutaneous administration of one of the following: (i) buffer alone, (ii) IL-7 alone (0.5 mg/kg), (iii) pegylated recombinant human G-CSF alone (5 μg), and (iv) IL-7 (0.5 mg/kg) in combination with G-CSF (5 μg). See FIG. 18A. Then, at day 3 post administration, both the number of LSK cells and HSCs in the peripheral blood were assessed using flow cytometry.

As shown in FIGS. 18B and 18D, compared to the control animals that received the buffer alone, there was an increased amount of LSK cells in animals treated with either the IL-7 protein or G-CSF. The number of LSK cells present in the peripheral blood of mice treated with just 0.5 mg/kg of IL-7 was comparable to that observed in mice treated with 5 μg of G-CSF, highlighting the potency of the IL-7 protein disclosed herein. The greatest number of LSK cells was observed in mice treated with the combination of IL-7 and G-CSF. The increase in the number of LSK cells correlated with an increase the number of HSCs (i.e., the greatest number of HSCs was observed in the combination group) (see FIGS. 18C and 18D).

The above results demonstrate that IL-7 proteins disclosed herein can greatly improve the mobilization of HSCs observed with G-CSF alone. The results further highlight the potential therapeutic effects of the combination of IL-7 and G-CSF, compared to treatment with either protein alone.

### Example 11: Effect of IL-7 and AMD3100 Combination on HSC Mobilization into Peripheral Blood

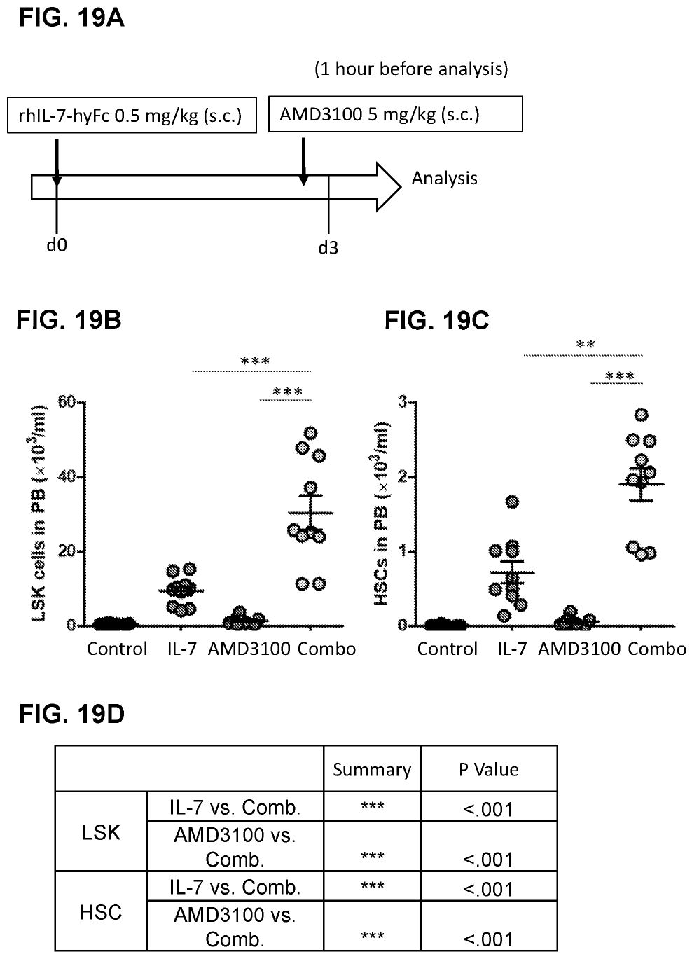

As described herein, AMD3100 (CXCR4 antagonist) is another regimen that is commonly used to promote the migration of HSCs from the bone marrow into the peripheral blood. Cashen et al., Future Oncol 3(1): 19-27 (February 2007). To determine whether IL-7 can also enhance the HSC mobilization effects of AMD3100, C57BL/6 animals received a single subcutaneous administration of the following: (i) buffer alone, (ii) IL-7 alone (0.5 mg/kg), (iii) AMD3100 (5 mg/kg), and (iv) IL-7 (0.5 mg/kg) in combination with AMD3100 (5 mg/kg). As shown in FIG. 19A, IL-7 protein was administered at day 0, while AMD3100 was administered 1 hour prior to analysis. At day 3 post initial administration, the number of both LSK cells and HSCs were again assessed in the peripheral blood using flow cytometry.

Similar to the earlier results (see Example 3), an increased amount of both LSK cells and HSCs were observed in animals that received a single administration of 0.5 mg/kg of the IL-7 protein (see FIGS. 19B and 19C, respectively). In animals treated with a single administration of 5 mg/kg of AMD3100, the number of LSK cells and HSCs were comparable to that of the control animals, suggesting that the administered dose of AMD3100 was not sufficient to induce an observable effect on HSC mobilization. And, in mice that received the combination of IL-7 protein and AMD3100, the greatest effect on HSC mobilization was observed. Interestingly, the increased number of LSK cells observed in the peripheral blood of animals from the combination group was greater than the sum of the amount of LSK cells observed in animals treated with IL-7 protein alone and AMD3100 alone, suggesting a synergistic effect (see FIGS. 19B, compare “comb.” vs. “IL-7”+“AMD3100”). Similar results were observed for HSCs (see FIG. 19C).

### Example 12: Effect of IL-7, G-CSF, and AMD3100 Triple Combination on HSC Mobilization into Peripheral Blood

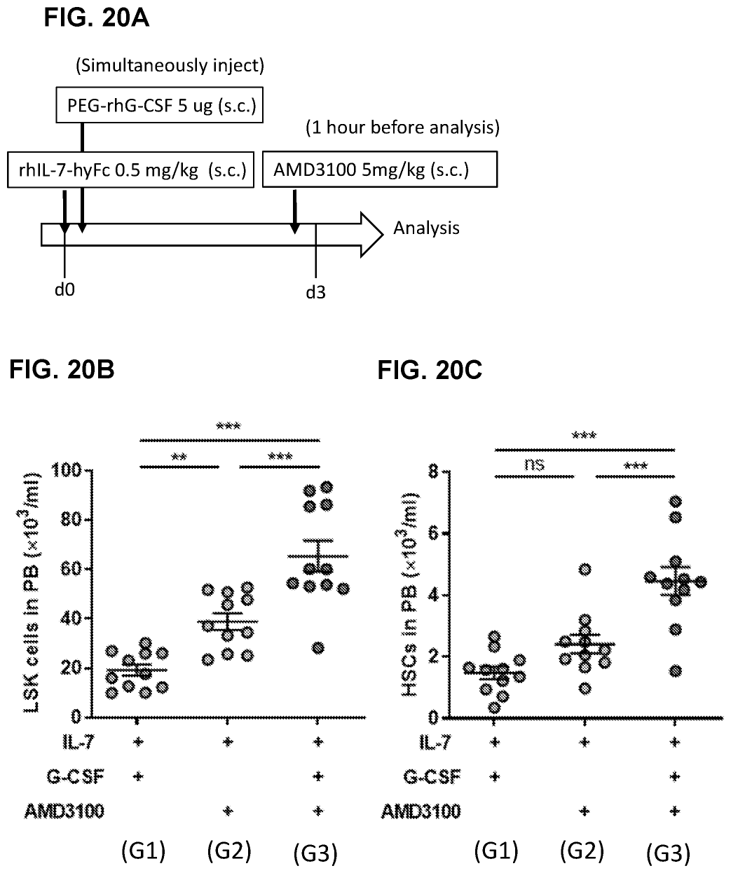

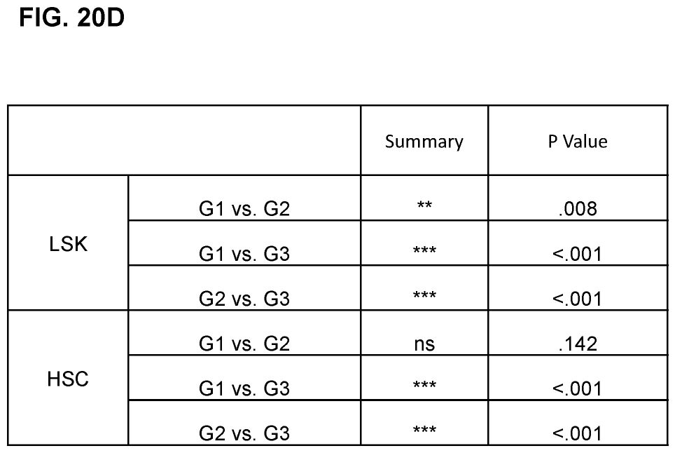

To assess whether the combination of IL-7, G-CSF, and AMD3100 can further enhance the mobilization of HSCs into the peripheral blood, C57BL/6 mice were treated with a single subcutaneous administration of the following: (i) IL-7 in combination with G-CSF (“G1”), (ii) IL-7 in combination with AMD3100 (“G2”), or (iii) IL-7 in combination with both G-CSF and AMD3100 (“G3”). As shown in FIG. 20A, both IL-7 (0.5 mg/kg) and G-CSF (5 μg) were administered at day 0. AMD3100 (5 mg/kg) was administered 1 hour prior to analysis.

As shown in FIGS. 20B-20D, the greatest effect on HSC mobilization was observed in the triple combination group. Collectively, the above results demonstrate that the IL-7 protein disclosed herein can greatly improve the effects observed with the currently used regimens for mobilizing HSCs, as well as other LSK cell subsets, into the peripheral blood.

What is claimed is:

1. A method for mobilizing a population of hematopoietic stem and progenitor (LSK) cells from a bone marrow into a peripheral blood of a subject in need thereof, comprising administering to the subject an effective amount of an IL-7 protein.

2. The method of claim 1, wherein the administering of the IL-7 protein increases the mobilization of the population of LSK cells from the bone marrow into the peripheral blood.

3. The method of claim 2, wherein the increase in the mobilization of the population of LSK cells results in an increase in the number of LSK cells in the peripheral blood of the subject.

4. The method of claim 3, wherein the number of LSK cells in the peripheral blood of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, or at least about 100-fold or more, compared to a reference (e.g., number of LSK cells in the peripheral blood of a corresponding subject that did not receive an administration of the IL-7 protein).

5. A method for increasing a mobilization of a population of hematopoietic stem and progenitor (LSK) cells from a bone marrow into a peripheral blood of a subject in need thereof, comprising administering to the subject an effective amount of an IL-7 protein in combination with an additional agent.

6. The method of claim 5, wherein the additional agent comprises a G-CSF, CXCR4 antagonist (e.g., AMD3100, POL6326, TG-0054, LY2510924, ALX-0651), CXCR2 antagonist (e.g., bortezomib, Groβ), anti-SDF-1 (e.g., BKT140), GM-CSF, IL-3, GM-CSF/IL-3 fusion proteins, FLK-2/FLT-3 ligand (e.g., CDX-301), stem cell factor, IL-6, IL-11, TPO, VEGF, VLA-4 antagonist (e.g., natalizumab), non-steroidal anti-inflammatory drug (e.g., meloxicam), PTH receptor agonist, TPO receptor agonist (e.g., eltrombopag) plerixafor, chemotherapy, or combinations thereof.

7. The method of claim 6, wherein the additional agent comprises G-CSF, AMD3100, or both.

8. The method of any one of claims 5 to 7, wherein the IL-7 protein and the additional agent are administered concurrently.

9. The method of any one of claims 5 to 7, wherein the IL-7 protein and the additional agent are administered sequentially.

10. The method of claim 9, wherein the IL-7 protein is administered prior to the administration of the additional agent.

11. The method of claim 9, wherein the IL-7 protein is administered after the administration of the additional agent.

12. The method of any one of claims 5 to 11, wherein the increase in the mobilization of the population of LSK cells results in an increase in the number of LSK cells in the peripheral blood of the subject.

13. The method of claim 12, wherein the number of LSK cells in the peripheral blood of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, or at least about 100-fold or more, compared to a reference (e.g., a corresponding subject that received the IL-7 protein alone or the additional agent alone).

14. The method of any one of claims 1 to 13, wherein the LSK cells comprise hematopoietic stem cells (HSCs), short-term HSCs (ST-HSCs), hematopoietic progenitor cell-2 (HPC-2), multipotent progenitors (MPPs), lymphoid-primed progenitor cells (LMPPs), common lymphoid progenitor cells (CLPs), common myeloid progenitor cells (CMPs), granulocyte-monocyte progenitor cells (GMPs), megakaryocyte-erythrocyte progenitor cells (MEPs), or combinations thereof.

15. The method of any one of claims 1 to 14, wherein the subject suffers from a tumor.

16. The method of any one of claims 1 to 15, wherein the subject is in need of a HSC transplantation.

17. The method of any one of claims 1 to 14, wherein the subject is a healthy individual.

18. A method for reconstituting a hematopoietic compartment of a subject having been treated with a therapy that is capable of depleting the hematopoietic compartment of the subject, comprising administering to the subject an effective amount of an IL-7 protein prior to treatment with the therapy, wherein the IL-7 protein is capable of inducing a mobilization of a population of hematopoietic stem and progenitor (LSK) cells from a bone marrow to a peripheral blood of the subject.

19. The method of claim 18, wherein the therapy comprises a chemotherapy, radiation therapy, or both.

20. The method of claim 18 or 19, further comprising administering an additional agent to the subject prior to treatment with the therapy.

21. The method of claim 20, wherein the additional agent comprises a G-CSF, CXCR4 antagonist (e.g., AMD3100, POL6326, TG-0054, LY2510924, ALX-0651), CXCR2 antagonist (e.g., bortezomib, Groβ), anti-SDF-1 (e.g., BKT140), GM-CSF, IL-3, GM-CSF/IL-3 fusion proteins, FLK-2/FLT-3 ligand (e.g., CDX-301), stem cell factor, IL-6, IL-11, TPO, VEGF, VLA-4 antagonist (e.g., natalizumab), non-steroidal anti-inflammatory drug (e.g., meloxicam), PTH receptor agonist, TPO receptor agonist (e.g., eltrombopag) plerixafor, chemotherapy, or combinations thereof.

22. The method of claim 21, wherein the additional agent comprises G-CSF, AMD3100, or both.

23. The method of any one of claims 20 to 22, wherein the IL-7 protein and the additional agent are administered concurrently.

24. The method of any one of claims 20 to 22, wherein the IL-7 protein and the additional agent are administered sequentially.

25. The method of claim 24, wherein the IL-7 protein is administered prior to the administration of the additional agent.

26. The method of claim 24, wherein the IL-7 protein is administered after the administration of the additional agent.

27. The method of any one of claims 18 to 26, further comprising isolating the population of LSK cells that have mobilized into the peripheral blood prior to treatment with the therapy.

28. The method of claim 27, wherein the isolated population of LSK cells are further expanded ex vivo.

29. The method of claim 27 or 28, further comprising infusing the isolated population of LSK cells to the subject after treatment with the therapy, wherein the infusion of the isolated population of LSK cells is capable of reconstituting the hematopoietic compartment of the subject.

30. The method of any one of claims 18 to 29, wherein the LSK cells comprise hematopoietic stem cells (HSCs), short-term HSCs (ST-HSCs), hematopoietic progenitor cell-2 (HPC-2), multipotent progenitors (MPPs), lymphoid-primed progenitor cells (LMPPs), common lymphoid progenitor cells (CLPs), common myeloid progenitor cells (CMPs), granulocyte-monocyte progenitor cells (GMPs), megakaryocyte-erythrocyte progenitor cells (MEPs), or combinations thereof.

31. The method of any one of claims 1 to 30, wherein the population of LSK cells that have mobilized into the peripheral blood are capable of long-term self-renewal.

32. The method of claim 31, wherein the population of LSK cells that have mobilized into the peripheral blood maintain the ability to self-renew for at least about one week, at least about two weeks, at least about three weeks, at least about one month, at least about two months, at least about three months, at least about four months, at least about five months, at least about six months, at least about seven months, at least about eight months, at least about nine months, at least about 10 months, at least about 11 months, or at least about one year or more.

33. The method of any one of claims 1 to 32, wherein the population of LSK cells that have mobilized into the peripheral blood are capable of differentiating into myeloid cells, lymphoid cells (e.g., T cells and/or B cells), or both.

34. A method for increasing an amount of hematopoietic stem and progenitor (LSK) cells in a peripheral blood of a subject in need thereof, comprising administering to the subject an effective amount of an IL-7 protein.

35. The method of claim 33, wherein the amount of LSK cells in the peripheral blood of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, or at least about 100-fold or more, compared to a reference (e.g., number of LSK cells in the peripheral blood of a corresponding subject that did not receive an administration of the IL-7 protein).

36. A method for increasing an amount of hematopoietic stem and progenitor (LSK) cells in a peripheral blood of a subject in need thereof, comprising administering to the subject an effective amount of an IL-7 protein in combination with an additional agent.

37. The method of claim 36, wherein the additional agent comprises a G-CSF, CXCR4 antagonist (e.g., AMD3100, POL6326, TG-0054, LY2510924, ALX-0651), CXCR2 antagonist (e.g., bortezomib, Groβ), anti-SDF-1 (e.g., BKT140), GM-CSF, IL-3, GM-CSF/IL-3 fusion proteins, FLK-2/FLT-3 ligand (e.g., CDX-301), stem cell factor, IL-6, IL-11, TPO, VEGF, VLA-4 antagonist (e.g., natalizumab), non-steroidal anti-inflammatory drug (e.g., meloxicam), PTH receptor agonist, TPO receptor agonist (e.g., eltrombopag) plerixafor, chemotherapy, or combinations thereof.

38. The method of claim 37, wherein the additional agent comprises G-CSF, AMD3100, or both.

39. The method of any one of claims 36 to 38, wherein the IL-7 protein and the additional agent are administered concurrently.

40. The method of any one of claims 36 to 38, wherein the IL-7 protein and the additional agent are administered sequentially.

41. The method of claim 40, wherein the IL-7 protein is administered prior to the administration of the additional agent.

42. The method of claim 40, wherein the IL-7 protein is administered after the administration of the additional agent.

43. The method of any one of claims 36 to 42, wherein the amount of LSK cells in the peripheral blood of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, or at least about 100-fold or more, compared to a reference (e.g., a corresponding subject that received the IL-7 protein alone or the additional agent alone).

44. The method of any one of claims 34 to 43, wherein the LSK cells comprise hematopoietic stem cells (HSCs), short-term HSCs (ST-HSCs), hematopoietic progenitor cell-2 (HPC-2), multipotent progenitors (MPPs), lymphoid-primed progenitor cells (LMPPs), common lymphoid progenitor cells (CLPs), common myeloid progenitor cells (CMPs), granulocyte-monocyte progenitor cells (GMPs), megakaryocyte-erythrocyte progenitor cells (MEPs), or combinations thereof.

45. The method of any one of claims 34 to 44, wherein the subject suffers from a tumor.

46. The method of any one of claims 34 to 45, wherein the subject is in need of a HSC transplant.

47. The method of any one of claims 34 to 44, wherein the subject is a healthy individual.

48. A method of treating a disease or disorder in a subject in need thereof, comprising administering to the subject an IL-7 protein and a therapy, wherein the IL-7 protein is capable of inducing a mobilization of a population of hematopoietic stem and progenitor (LSK) cells from a bone marrow to a peripheral blood of the subject.

49. The method of claim 48, wherein the IL-7 protein is administered to the subject prior to the therapy.

50. The method of claim 48, wherein an amount of the LSK cells in the peripheral blood of the subject is increased compared to a reference (e.g., a corresponding subject that did not receive an administration of the IL-7 protein).

51. The method of claim 50, wherein the amount of LSK cells in the peripheral blood of the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 50-fold, at least about 75-fold, or at least about 100-fold or more, compared to the reference.

52. The method of any one of claims 48 to 51, further comprising isolating the LSK cells from the peripheral blood of the subject prior to the therapy.

53. The method of claim 52, wherein the therapy is capable of depleting and/or reducing the number of one or more blood cells in the subject.

54. The method of claim 53, wherein the one or more blood cells comprise a myeloid cell, lymphoid cell, or both.

55. The method of claim 54, wherein the myeloid cell comprises a monocyte, macrophage, dendritic cells, mast cells, neutrophil, basophil, eosinophil, erythrocyte, megakaryocyte, or combinations thereof.

56. The method of claim 54 or 55, wherein the lymphoid cell comprises an innate lymphoid cell, natural killer cell, T lymphocyte, B lymphocyte, or combinations thereof.

57. The method of any one of claims 53 to 56, wherein the therapy comprises a chemotherapy, radiation therapy, immunotherapy, serotherapy, targeted therapy (e.g., anti-thymocyte immunoglobulin), or combinations thereof.

58. The method of any one of claims 53 to 57, further comprising infusing the isolated LSK cells to the subject after the administration of the therapy.

59. The method of any one of claims 48 to 58, wherein the LSK cells comprise hematopoietic stem cells (HSCs), short-term HSCs (ST-HSCs), hematopoietic progenitor cell-2 (HPC-2), multipotent progenitors (MPPs), lymphoid-primed progenitor cells (LMPPs), common lymphoid progenitor cells (CLPs), common myeloid progenitor cells (CMPs), granulocyte-monocyte progenitor cells (GMPs), megakaryocyte-erythrocyte progenitor cells (MEPs), or combinations thereof.

60. The method of any one of claims 48 to 59, wherein the disease or disorder comprises an acute myeloid leukaemia, acute lymphoblastic leukaemia, chronic myelogenous leukaemia, chronic lymphocytic leukaemia, myelofibrosis, myelodysplastic syndromes, diffuse large B cell lymphoma, follicular lymphoma, mantle cell lymphoma, Waldenström macroglobulinemia, peripheral T cell lymphoma, primary cutaneous T cell lymphoma, Hodgkin lymphoma, multiple myeloma, amyloidosis, juvenile myelomonocytic leukaemia, Non-Hodgkin lymphoma, breast cancer, germ cell tumours, ovarian cancer, medulloblastoma, small cell lung cancer, soft tissue sarcoma, ewing's sarcoma, renal cell cancer, pancreatic cancer, colorectal cancer, multiple sclerosis, systemic sclerosis, systemic lupus erythematosus, crohn's disease, rheumatoid arthritis, juvenile idiopathic arthritis, monogenic autoimmune disorders, vasculitis, polymyositis-dermatomyositis, autoimmune cytopenias, neuromyelitis optica, chronic inflammatory demyelinating polyneuropathy, myasthenia gravis and stiff person syndrome, type 1 diabetes, refractory coeliac disease type II, sarcoma, neuroblastoma, brain tumours, Wilms' tumor, or any combination thereof.

61. The method of any one of claims 1 to 50, wherein the subject is a human.

62. The method of any one of claims 1 to 61, wherein the IL-7 protein is not a wild-type IL-7 protein.

63. The method of any one of claims 1 to 62, wherein the IL-7 protein is a fusion protein.

64. The method of any one of claims 1 to 63, wherein the IL-7 protein comprises an oligopeptide consisting of 1 to 10 amino acid residues.

65. The method of claim 64, wherein the oligopeptide comprises methionine (M), glycine (G), methionine-methionine (MM), glycine-glycine (GG), methionine-glycine (MG), glycine-methionine (GM), methionine-methionine-methionine (MMM), methionine-methionine-glycine (MMG), methionine-glycine-methionine (MGM), glycine-methionine-methionine (GMM), methionine-glycine-glycine (MGG), glycine-methionine-glycine (GMG), glycine-glycine-methionine (GGM), glycine-glycine-glycine (GGG), methionine-glycine-glycine-methionine (MGGM) (SEQ ID NO: 41), methionine-methionine-glycine-glycine (MMGG) (SEQ ID NO: 42), glycine-glycine-methionine-methionine (GGMM) (SEQ ID NO: 43), methionine-glycine-methionine-glycine (MGMG) (SEQ ID NO: 44), glycine-methionine-methionine-glycine (GMMG) (SEQ ID NO: 45), glycine-glycine-glycine-methionine (GGGM) (SEQ ID NO: 46), methionine-glycine-glycine-glycine (MGGG) (SEQ ID NO: 47), glycine-methionine-glycine-glycine (GMGG) (SEQ ID NO: 48), glycine-glycine-methionine-glycine (GGMG) (SEQ ID NO: 49), glycine-glycine-methionine-methionine-methionine (GGMMM) (SEQ ID NO: 50), glycine-glycine-glycine-methionine-methionine (GGGMM) (SEQ ID NO: 51), glycine-glycine-glycine-glycine-methionine (GGGGM) (SEQ ID NO: 52), methionine-glycine-methionine-methionine-methionine (MGMMM) (SEQ ID NO: 53), methionine-glycine-glycine-methionine-methionine (MGGMM) (SEQ ID NO: 54), methionine-glycine-glycine-glycine-methionine (MGGGM) (SEQ ID NO: 55), methionine-methionine-glycine-methionine-methionine (MMGMM) (SEQ ID NO: 56), methionine-methionine-glycine-glycine-methionine (MMGGM) (SEQ ID NO: 57), methionine-methionine-glycine-glycine-glycine (MMGGG) (SEQ ID NO: 58), methionine-methionine-methionine-glycine-methionine (MMMGM) (SEQ ID NO: 59), methionine-glycine-methionine-glycine-methionine (MGMGM) (SEQ ID NO: 60), glycine-methionine-glycine-methionine-glycine (GMGMG) (SEQ ID NO: 61), glycine-methionine-methionine-methionine-glycine (GMMMG) (SEQ ID NO: 62), glycine-glycine-methionine-glycine-methionine (GGMGM) (SEQ ID NO: 63), glycine-glycine-methionine-methionine-glycine (GGMMG) (SEQ ID NO: 64), glycine-methionine-methionine-glycine-methionine (GMMGM) (SEQ ID NO: 65), methionine-glycine-methionine-methionine-glycine (MGMMG) (SEQ ID NO: 66), glycine-methionine-glycine-glycine-methionine (GMGGM) (SEQ ID NO: 67), methionine-methionine-glycine-methionine-glycine (MMGMG) (SEQ ID NO: 68), glycine-methionine-methionine-glycine-glycine (GMMGG) (SEQ ID NO: 69), glycine-methionine-glycine-glycine-glycine (GMGGG) (SEQ ID NO: 70), glycine-glycine-methionine-glycine-glycine (GGMGG) (SEQ ID NO: 71), glycine-glycine-glycine-glycine-glycine (GGGGG) (SEQ ID NO: 72), or combinations thereof.

66. The method of claim 65, wherein the oligopeptide is methionine-glycine-methionine (MGM).

67. The method of any one of claims 1 to 66, wherein the IL-7 protein comprises a half-life extending moiety.

68. The method of claim 67, wherein the half-life extending moiety comprises an Fc, albumin, an albumin-binding polypeptide, Pro/Ala/Ser (PAS), a C-terminal peptide (CTP) of the β subunit of human chorionic gonadotropin, polyethylene glycol (PEG), long unstructured hydrophilic sequences of amino acids (XTEN), hydroxyethyl starch (HES), an albumin-binding small molecule, or a combination thereof.

69. The method of claim 68, wherein the half-life extending moiety is an Fc.

70. The method of claim 69, wherein the Fc is a hybrid Fc, comprising a hinge region, a CH2 domain, and a CH3 domain,
wherein the hinge region comprises a human IgD hinge region,
wherein the CH2 domain comprises a part of human IgD CH2 domain and a part of human IgG4 CH2 domain, and
wherein the CH3 domain comprises a part of human IgG4 CH3 domain.

71. The method of any one of claims 1 to 70, wherein the IL-7 protein comprises an amino acid sequence having a sequence identity of at least about 70%, at least about 75%, at least about 80%, at least about 85%, at least about 90%, at least about 95%, at least about 96%, at least about 97%, at least about 98%, at least about 99%, or about 100% to SEQ ID NOs: 1-6 and 15-25.

72. The method of any one of claims 1 to 71, wherein the IL-7 protein is administered at a dose between about 20 μg/kg and about 600 μg/kg.

73. The method of claim 72, wherein the IL-7 protein is administered at a dose of about 60 μg/kg.

74. The method of any one of claims 1 to 73, wherein the IL-7 protein is administered at a dosing frequency of about once a week, about once in two weeks, about once in three weeks, about once in four weeks, about once in five weeks, about once in six weeks, about once in seven weeks, about once in eight weeks, about once in nine weeks, about once in 10 weeks, about once in 11 weeks, or about once in 12 weeks.

75. The method of any one of claims 1 to 74, wherein the IL-7 protein is administered to the subject parenthetically, intramuscularly, subcutaneously, ophthalmic, intravenously, intraperitoneally, intradermally, intraorbitally, intracerebrally, intracranially, intraspinally, intraventricular, intrathecally, intracistemally, intracapsularly, intratumorally, or any combination thereof.

76. The method of any one of claims 1 to 75, further comprising administering at least one additional therapeutic agent to the subject.
